(19) United States
(12) Patent Application Publication
(10) Pub. No.: US 2006/0134221 A1
(43) Pub. Date: Jun. 22, 2006

(54) Methods for producing block copolymer/amphiphilic particles

(75) Inventor: Andrew Geall, San Marcos, CA (US)

Correspondence Address: STERNE, KESSLER, GOLDSTEIN & FOX PLLC, 1100 NEW YORK AVENUE, N.W., WASHINGTON, DC, 20005, US

(73) Assignee: Vical Incorporated, San Diego, CA (US)

(21) Appl. No.: 11/292,280

(22) Filed: Dec. 2, 2005

Related U.S. Application Data
(60) Provisional application No. 60/632,612, filed on Dec. 3, 2004.

(51) Int. Cl. A61K 9/14

(52) U.S. Cl. 424/489

(57) ABSTRACT

The invention relates to a method for manufacturing cell delivery particles, pharmaceutical component-particle dispersions, compositions comprising cell delivery particles and pharmaceutical compositions comprising pharmaceutical component-particle dispersions. The method comprises homogenization of mixtures comprising amphiphilic components and a block copolymer to form stable particles. The invention is also directed to cell delivery particles and pharmaceutical component-particle dispersions produced by the claimed methods and compositions comprising same. In certain embodiments, the cell delivery particles may further comprise co-lipids. The invention further relates to methods of generating an immune response, treating or preventing a disease or condition, or delivering a biologically active molecule to cells in vitro comprising administration of the pharmaceutical compositions described herein.

### CROSS-REFERENCE TO RELATED APPLICATIONS

The present application claims the benefit of U.S. Provisional Application No. 60/632,612, filed Dec. 3, 2004, which is incorporated herein by reference in its entirety.

### BACKGROUND OF THE INVENTION

1. Field of the Invention

The present invention relates to methods for producing cell delivery particles comprising a block copolymer and an amphiphilic component. Additionally, the invention relates to methods for producing pharmaceutical compositions comprising pharmaceutical component-particles dispersions. The invention also relates to the cell delivery particles and compositions comprising the cell delivery particles, as well as pharmaceutical compositions and pharmaceutical component-particles dispersions, produced by the methods described herein. In certain embodiments, the particles and compositions of the present invention may contain additional components such as co-lipids and agents to aid in the lyophilization of the pharmaceutical compositions. A further aspect of the invention include methods for treating or preventing a disease or condition, methods for generating a detectable immune response and a method for delivering to cells, in vitro, a pharmaceutical component.

2. Background Art

The delivery of biologically active molecules such as drugs, hormones, enzymes, nucleic acids and antigens, including viruses, to cells in vitro or in vivo is of great interest for potential pharmaceutical uses such as immune response induction and modulation, therapeutic polypeptide delivery, and amelioration of genetic defects as well as research applications. For example, a polynucleotide may encode an antigen that induces an immune response against an infectious pathogen or against tumor cells (Restifo, N. P. et al., Folia Biol. 40:74-88 (1994); Ulmer, J. B. et al., Ann. NY Acad. Sci. 772:117-125 (1995); Horton, H. M. et al., Proc. Natl. Acad. Sci. USA 96:1553-1558 (1999); Yagi, K. et al., Hum. Gene Ther. 10: 1975-1982 (1999)). The polynucleotide may encode an immunomodulatory polypeptide, e.g., a cytokine, that diminishes an immune response against self antigens or modifies the immune response to foreign antigens, allergens, or transplanted tissues (Qin, L. et al., Ann. Surg. 220:508-518 (1994); Dalesandro, J. et al., J. Thorac. Cardiovasc. Surg. 111: 416-421 (1996); Moffatt, M. and Cookson, W., Nat. Med. 2:515-516 (1996); Ragno, S. et al., Arth. and Rheum. 40:277-283 (1997); Dow, S. W. et al., Hum. Gene Ther. 10:1905-1914 (1999); Piccirillo, C. A. et al., J. Immunol. 161:3950-3956 (1998); Piccirillo, C. A. and Prud'homme, G. J., Hum. Gene Ther. 10: 915-1922 (1999)). For therapeutic polypeptide delivery, the polynucleotide may encode, for example, an angiogenic protein, hormone, growth factor, or enzyme (Levy, M. Y. et al., Gene Ther. 3:201-211 (1996); Tripathy, S. K. et al., Proc. Natl. Acad. Sci. USA 93:10876-10880 (1996); Tsurumi, Y. et al., Circulation 94:3281-3290 (1996); Novo, F. J. et al., Gene Ther. 4:488492 (1997); Baumgartner, I. et al., Circulation 97:1114-1123 (1998); Mir, L. M. et al., Proc. Natl. Acad. Sci. USA 96:4262-4267 (1999)). For amelioration of genetic defects, the polynucleotide may encode normal copies of defective proteins such as dystrophin or cystic fibrosis transmembrane conductance regulator (Danko, I. et al., Hum. Mol. Genet. 2:2055-2061 (1993); Cheng, S. H. and Scheule, R. K., Adv. Drug Deliv. Rev. 30:173-184 (1998)).

U.S. Pat. No. 5,656,611 and Published International Patent Application No. WO 99/06055 disclose compositions which include a polynucleotide, and a block copolymer containing a non-ionic portion and a polycationic portion. A surfactant is added to increase solubility and the end result is the formation of micelles. This formulation allows stabilization of polynucleic acids and enhances transfection efficiency. Published International Patent Application No. WO 99/21591 discloses a soluble ionic complex comprising an aqueous mixture of a polynucleotide and a benzylammonium group-containing cationic surfactant and the use of this complex in vaccine and gene delivery.

U.S. Pat. Nos. 6,120,794 and 6,586,003 B2 described methods for creating emulsions comprising a cationic amphiphilic component and a non-ionic surfactant component which form micelles in an aqueous solution. The methods described include combining the cationic amphiphilic component and a nonionic surfactant and optionally a neutral phospholipid in an organic solvent, followed by the removal of the organic solvent to leave a lipid film and then suspending the lipid film in an aqueous carrier. The methods described herein do not require organic solvents or their removal. All components are in aqueous solution prior to homogenization to create the particles of the invention.

Published International Patent Application No. WO 02/00844, hereby incorporated in its entirety by reference, describes polynucleotide vaccine adjuvants which comprise a polynucleotide, a block copolymer and a cationic surfactant. By including the cationic surfactant in the formulation, the percentage of polynucleotide that is associated with the block copolymer/cationic surfactant adjuvant is increased. In addition, this formulation has demonstrated enhanced in vivo immune response to polynucleotide vaccines and/or gene therapy-based transgenes.

The method described in Published International Patent Application No. WO 02/00844 requires thermally cycling the polynucleotide/block copolymer/cationic surfactant composition mixture several times through the cloud point of the block copolymer to form the polynucleotide complexes. These multiple heating and cooling cycles are expensive and time consuming, especially when considering the production of large quantities of the formulation required during commercial manufacturing. In addition, no sterilization step was disclosed in WO 02/00844. The requirement to sterilize all components prior to mixing and producing the formulation under sterile conditions increases the cost of large-scale production considerably and hinders the ability to scale up the production of this formulation for commercial manufacturing.

Furthermore, the method described in WO 02/00844 is limited by the concentration of cationic amphiphile and what cationic amphiphile can be used as the cationic surfactant. The method as described requires thermal cycling below the cloud point of the block copolymer. At temperatures below the cloud point of many block copolymers, certain cationic amphiphiles are insoluble, particularly cationic amphiphiles with longer alkyl chains. These molecules are insoluble below the point of many block copolymers and as a result do not form particles comprising the block copolymer. Furthermore, cationic amphiphiles with intermediate length alkyl chains may be soluble at temperature below the cloud point of many block copolymers only at low concentrations. At higher concentrations, the cationic amphiphile may precipitate out of solution.

Therefore, a need remains in the art for a method of producing compositions comprising a block copolymer and a amphiphilic component that is amenable to all combinations of amphiphiles and block copolymers and which also allow for a scalable production platform.

The methods of the present invention provide for the convenience of processing without multiple temperature steps and allows one of skill in the art to produce formulations particularly suited for their experimental or therapeutic use, many of which could not have been manufactured by prior methods.

### BRIEF SUMMARY OF THE INVENTION

The present invention is directed to methods for manufacturing cell delivery particles comprising a block copolymer and an amphiphilic component via homogenization in an aqueous solution. The method results in the formation of a homogenate which is in the form of particles. The method allows for the production of particles using amphiphilic components which could not be incorporated into stable particles by previously described methods. The present invention is also directed to methods for manufacturing a pharmaceutical component-particle dispersion comprising cell delivery particles and a pharmaceutical component which may include, but is not limited to, a pharmaceutically active drug, an antigenic molecule, a polynucleotide or any combination thereof.

In a specific embodiment, the invention provides for methods of manufacturing cell delivery particles which include a block copolymer and any amphiphilic component comprising an amphiphile selected from the group consisting of: a cationic amphiphile, anionic amphiphile, neutral amphiphile or any combination thereof. In addition, certain embodiments of the present invention provide for methods of manufacturing cell delivery particles which additionally include co-lipids such as neutral lipids, charged lipids or combinations thereof.

The present invention further provides for cell delivery particles, pharmaceutical component-particle dispersions, cell delivery particle compositions and pharmaceutical compositions produced by the methods described herein. The pharmaceutical component-particle dispersions and pharmaceutical compositions comprising pharmaceutical component-particle dispersions, contain a pharmaceutical component (e.g. a pharmaceutically active drug, an antigenic molecule, a polynucleotide or any combination thereof).

In a specific embodiment, the pharmaceutical compositions of the present invention comprise cell delivery particles which comprise a block copolymer, amphiphilic component, an optional co-lipid and a polynucleotide which form a pharmaceutical component-particle dispersion.

Also within the scope of the present invention are methods relating to the lyophilization of the cell delivery particles, pharmaceutical component-particle dispersions, cell delivery particle compositions and pharmaceutical compositions comprising pharmaceutical component-particle dispersions. The lyophilization method involves a flash-freezing step at a temperature of about −200° C. to about −150° C. and then lyophilization of the frozen cell delivery particles, pharmaceutical component-particle dispersions, cell delivery particle compositions and pharmaceutical compositions comprising pharmaceutical component-particle dispersions. The lyophilization may occur in several steps, at different temperatures and over different periods of time. In certain embodiments, the cell delivery particles, pharmaceutical component-particle dispersions, cell delivery particle compositions and pharmaceutical compositions comprising pharmaceutical component-particle dispersions to be lyophilized may further comprise a cryoprotectant. The invention further provides for the lyophilized cell delivery particles, pharmaceutical component-particle dispersions, cell delivery particle compositions and pharmaceutical compositions comprising pharmaceutical component-particle dispersions which have been reconstituted in an aqueous solution.

The present invention further provides for a method of enhancing or generating an immune response in a vertebrate comprising administering the cell delivery particles, pharmaceutical component-particle dispersions, cell delivery particle compositions and pharmaceutical compositions comprising pharmaceutical component-particle dispersions of the present invention. Additionally, the invention provides for a method for treating or preventing a disease or condition in a vertebrate as well as methods for delivering to a cell in vitro a pharmaceutical component via the administration of the cell delivery particles, pharmaceutical component-particle dispersions, cell delivery particle compositions and pharmaceutical compositions comprising pharmaceutical component-particle dispersions described herein.

Additionally, the invention is directed to kits comprising cell delivery particles, pharmaceutical component-particle dispersions, cell delivery particle compositions and pharmaceutical compositions comprising pharmaceutical component-particle dispersions of the present invention.

### BRIEF DESCRIPTION OF THE DRAWINGS/FIGURES

FIG. 1 is a schematic diagram of the thermal cycling method described in U.S. Published Application 2004/0162256 A1.

FIG. 2 is a schematic diagram of the thermal cycling method with a cold filtration step described in U.S. Published Application 2004/0162256 A1.

FIG. 3 illustrates the structures of the alkyl chain homologs which comprise benzalkonium chloride solutions BTC 50 NF and BTC 65 NF.

FIG. 4 is a schematic drawing of the Avestin EmulsiFlex-C50 high pressure homogenizer.

### DETAILED DESCRIPTION OF THE INVENTION

The present invention includes methods for manufacturing compositions, e.g., for the delivery of pharmaceutical components in vivo and in vitro. The methods result in the production of particles which comprise a block copolymer and an amphiphilic component. The methods of the present invention allow for the production of stable particles with various amphiphilic components which could not have been produced by methods previously described in the art. Specifically, the invention is directed to methods for manufacturing cell delivery particles and pharmaceutical compositions comprising pharmaceutical component-particle dispersions. The invention is also related to the particles, compositions comprising the particles, pharmaceutical component-particle dispersions and pharmaceutical compositions comprising the pharmaceutical component-particle dispersions produced by the claimed methods. In further embodiments the invention relates to methods for generating a detectable immune response, treating or preventing a disease or condition, and delivering to a cell in vitro a pharmaceutically active drug, an antigenic molecule or a polynucleotide by administration of the claimed pharmaceutical compositions. The invention is further directed to a kit comprising the pharmaceutical compositions produced by the claimed methods.

One embodiment of the present invention relates to a method for manufacturing cell delivery particles, comprising homogenizing, in an aqueous solution, a mixture comprising a block copolymer and an amphiphilic component, wherein the amphiphilic component comprises an amphiphile selected from the group consisting of: a cationic amphiphile, an anionic amphiphile, a neutral amphiphile or any combinations thereof. The mixture forms a homogenate which is comprised of particles formed from the block copolymer and amphiphilic component.

In an additional embodiment the cell delivery particles produced by the claimed methods are mixed with a pharmaceutical component selected from the group consisting of: a pharmaceutically active drug, an antigenic molecule, a polynucleotide or any combination thereof to form a pharmaceutical component-particle dispersion. Additionally, the cell delivery particles produced by the claimed methods further comprise a co-lipid (e.g. a neutral co-lipid).

The methods as described above may further comprise lyophilization. In this embodiment the resulting homogenate, in the form of particles, is flash frozen at a temperature from about −200° C. to about −150° C., followed by lyophilization of the frozen homogenate at various temperatures for varying amounts of time.

Alternative embodiments of the present invention include cell delivery compositions, pharmaceutical compositions, cell delivery particles and pharmaceutical component-particle dispersions produced by the claimed methods. Cell delivery compositions include cell delivery particles comprising a block copolymer and an amphiphilic component, wherein the amphiphilic component comprises an amphiphile selected from the group consisting of: a cationic amphiphile, an anionic amphiphile, a neutral amphiphile, or any combination thereof. Additionally, pharmaceutical compositions comprise pharmaceutical component-particle dispersions comprising cell delivery particles, as described above, and an additional pharmaceutical component selected from the group consisting of a pharmaceutically active drug, an antigenic molecule, a polynucleotide or combinations thereof. In certain embodiments the cell delivery particles comprise a block copolymer, an amphiphilic component and a co-lipid (e.g. a neutral co-lipid). In an additional embodiment the invention is directed to lyophilized cell delivery particles, pharmaceutical component-particle dispersions, cell delivery particle compositions and pharmaceutical compositions produced by the methods of the claimed invention and reconstituted forms thereof.

The invention is also directed to methods of generating a detectable immune response by administration to a vertebrate one or more cell delivery particles, pharmaceutical component-particle dispersions or pharmaceutical compositions comprising the same, produced by the claimed methods. The one or more cell delivery particles, pharmaceutical component-particle dispersions or pharmaceutical compositions comprising the same to be delivered typically contain pharmaceutical components which are administered in an amount sufficient elicit a detectable immune response. In certain embodiments the pharmaceutical component is a polynucleotide which encodes a polypeptide. Additionally, the invention is directed to methods of delivering a pharmaceutically active drug, an antigenic molecule or a polynucleotide to cells in vitro via administration of cell delivery particles, pharmaceutical component-particle dispersions or pharmaceutical compositions comprising the same. The invention is further directed to methods of treating or preventing a disease or condition in a vertebrate by administration of any of the claimed cell delivery particles, pharmaceutical component-particle dispersions or pharmaceutical compositions comprising the same.

It is to be noted that the term “a” or “an” entity, refers to one or more of that entity; for example “a co-lipid” is understood to represent one or more co-lipid molecules. As such, the terms “a” (or “an”), “one or more,” and “at least one” can be used interchangeably herein.

The term “eukaryote” or “eukaryotic organism” is intended to encompass all organisms in the animal, plant, and protist kingdoms, including protozoa, fungi, yeasts, green algae, single celled plants, multi celled plants, and all animals, both vertebrates and invertebrates. The term does not encompass bacteria or viruses. A “eukaryotic cell” is intended to encompass a singular “eukaryotic cell” as well as plural “eukaryotic cells,” and comprises cells derived from a eukaryote.

The term “vertebrate” is intended to encompass a singular “vertebrate” as well as plural “vertebrates,” and comprises mammals and birds, as well as fish, reptiles, and amphibians.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears. In certain embodiments, the mammal is a human subject.

The term “polynucleotide” is intended to encompass a singular nucleic acid or nucleic acid fragment as well as plural nucleic acids or nucleic acid fragments, and refers to an isolated molecule or construct, e.g., a virus genome (e.g., a non-infectious viral genome), messenger RNA (mRNA), plasmid DNA (pDNA), or derivatives of pDNA (e.g., minicircles as described in Darquet, A-M et al., Gene Therapy 4:1341-1349 (1997)) comprising a polynucleotide. A nucleic acid or fragment thereof may be provided in linear (e.g., mRNA), circular (e.g., plasmid), or branched form as well as double-stranded or single-stranded forms. A polynucleotide may comprise a conventional phosphodiester bond or a non-conventional bond (e.g., an amide bond, such as found in peptide nucleic acids (PNA)).

The terms “nucleic acid” or “nucleic acid fragment” refer to any one or more nucleic acid segments, e.g., DNA or RNA fragments, present in a polynucleotide or construct.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, and the like, are not part of a coding region. Two or more nucleic acids or nucleic acid fragments of the present invention can be present in a single polynucleotide construct, e.g., on a single plasmid, or in separate polynucleotide constructs, e.g., on separate (different) plasmids. Furthermore, any nucleic acid or nucleic acid fragment may encode a single polypeptide or fragment, derivative, or variant thereof, e.g., or may encode more than one polypeptide, e.g., a nucleic acid may encode two or more polypeptides. In addition, a nucleic acid may include a regulatory element such as a promoter, ribosome binding site, or a transcription terminator, or may encode heterologous coding regions fused to a coding region, e.g., specialized elements or motifs, such as a secretory signal peptide or a heterologous functional domain.

The terms “infectious polynucleotide” or “infectious nucleic acid” are intended to encompass isolated viral polynucleotides and/or nucleic acids which are solely sufficient to mediate the synthesis of complete infectious virus particles upon uptake by permissive cells. Thus, “infectious nucleic acids” do not require pre-synthesized copies of any of the polypeptides it encodes, e.g., viral replicases, in order to initiate its replication cycle in a permissive host cell.

The terms “non-infectious polynucleotide” or “non-infectious nucleic acid” as defined herein are polynucleotides or nucleic acids which cannot, without additional added materials, e.g, polypeptides, mediate the synthesis of complete infectious virus particles upon uptake by permissive cells. An infectious polynucleotide or nucleic acid is not made “non-infectious” simply because it is taken up by a non-permissive cell. For example, an infectious viral polynucleotide from a virus with limited host range is infectious if it is capable of mediating the synthesis of complete infectious virus particles when taken up by cells derived from a permissive host (i.e., a host permissive for the virus itself). The fact that uptake by cells derived from a non-permissive host does not result in the synthesis of complete infectious virus particles does not make the nucleic acid “non-infectious.” In other words, the term is not qualified by the nature of the host cell, the tissue type, or the species taking up the polynucleotide or nucleic acid fragment.

In some cases, an isolated infectious polynucleotide or nucleic acid may produce fully-infectious virus particles in a host cell population which lacks receptors for the virus particles, i.e., is non-permissive for virus entry. Thus viruses produced will not infect surrounding cells. However, if the supernatant containing the virus particles is transferred to cells which are permissive for the virus, infection will take place.

The terms “replicating polynucleotide” or “replicating nucleic acid” are meant to encompass those polynucleotides and/or nucleic acids which, upon being taken up by a permissive host cell, are capable of producing multiple, e.g., one or more copies of the same polynucleotide or nucleic acid. Infectious polynucleotides and nucleic acids are a subset of replicating polynucleotides and nucleic acids; the terms are not synonymous. For example, a defective virus genome lacking the genes for virus coat proteins may replicate, e.g., produce multiple copies of itself, but is NOT infectious because it is incapable of mediating the synthesis of complete infectious virus particles unless the coat proteins, or another nucleic acid encoding the coat proteins, are exogenously provided.

In certain embodiments, the polynucleotide, nucleic acid, or nucleic acid fragment is DNA. In the case of DNA, a polynucleotide comprising a nucleic acid which encodes a polypeptide normally also comprises a promoter and/or other transcription or translation control elements operably associated with the polypeptide-encoding nucleic acid fragment. An operable association is when a nucleic acid fragment encoding a gene product, e.g., a polypeptide, is associated with one or more regulatory sequences in such a way as to place expression of the gene product under the influence or control of the regulatory sequence(s). Two DNA fragments (such as a polypeptide-encoding nucleic acid fragment and a promoter associated with the 5′ end of the nucleic acid fragment) are “operably associated” if induction of promoter function results in the transcription of mRNA encoding the desired gene product and if the nature of the linkage between the two DNA fragments does not (1) result in the introduction of a frame-shift mutation, (2) interfere with the ability of the expression regulatory sequences to direct the expression of the gene product, or (3) interfere with the ability of the DNA template to be transcribed. Thus, a promoter region would be operably associated with a nucleic acid fragment encoding a polypeptide if the promoter were capable of effecting transcription of that nucleic acid fragment. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, elements from picornaviruses (particularly an internal ribosome entry site, or IRES, also referred to as a CITE sequence).

A DNA polynucleotide of the present invention may be a circular or linearized plasmid, or other linear DNA which may also be non-infectious and nonintegrating (i.e., does not integrate into the genome of vertebrate cells). A linearized plasmid is a plasmid that was previously circular but has been linearized, for example, by digestion with a restriction endonuclease. Linear DNA may be advantageous in certain situations as discussed, e.g., in Cherng, J. Y., et al., J. Control. Release 60:343-53 (1999), and Chen, Z. Y., et al. Mol. Ther. 3:403-10 (2001), both of which are incorporated herein by reference.

Alternatively, DNA virus genomes may be used to administer DNA polynucleotides into vertebrate cells. In certain embodiments, a DNA virus genome of the present invention is nonreplicative, noninfectious, and/or nonintegrating. Suitable DNA virus genomes include without limitation, herpes virus genomes, adenovirus genomes, adeno-associated virus genomes, and poxvirus genomes. References citing methods for the in vivo introduction of non-infectious virus genomes to vertebrate tissues are well known to those of ordinary skill in the art, and are cited supra.

In other embodiments, a polynucleotide of the present invention is RNA, for example, in the form of messenger RNA (mRNA) antisense RNA, short interfering RNA (siRNA), double-stranded RNA (dsRNA), transfer RNA (tRNA), ribosomal RNA (rRNA) and ribozymes.

Polynucleotides, nucleic acids, and nucleic acid fragments of the present invention may be associated with additional nucleic acids which encode secretory or signal peptides, which direct the secretion of a polypeptide encoded by a nucleic acid fragment or polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native leader sequence is used, or a functional derivative of that sequence that retains the ability to direct the secretion of the polypeptide that is operably associated with it. Alternatively, a heterologous mammalian leader sequence, or a functional derivative thereof, may be used. For example, the wild-type leader sequence may be substituted with the leader sequence of human tissue plasminogen activator (TPA) or mouse β-glucuronidase.

As used herein, the term “plasmid” refers to a construct made up of genetic material (i.e., nucleic acids). Typically a plasmid contains an origin of replication which is functional in bacterial host cells, e.g., Escherichia coli, and selectable markers for detecting bacterial host cells comprising the plasmid. Plasmids of the present invention may include genetic elements as described herein arranged such that an inserted coding sequence can be transcribed and translated in eukaryotic cells. Also, the plasmid may include a sequence from a viral nucleic acid. However, such viral sequences normally are not sufficient to direct or allow the incorporation of the plasmid into a viral particle, and the plasmid is therefore a non-viral vector. In certain embodiments described herein, a plasmid is a closed circular DNA molecule.

The term “expression” refers to the biological production of a product encoded by a coding sequence. In most cases a DNA sequence, including the coding sequence, is transcribed to form a messenger-RNA (mRNA). The messenger-RNA is then translated to form a polypeptide product which has a relevant biological activity. Also, the process of expression may involve further processing steps to the RNA product of transcription, such as splicing to remove introns, and/or post-translational processing of a polypeptide product.

As used herein, the term “polypeptide” is intended to encompass a singular “polypeptide” as well as plural “polypeptides,” and comprises any chain or chains of two or more amino acids. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” may be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

Also included as polypeptides of the present invention are fragments, derivatives, analogs or variants of the foregoing polypeptides, and any combination thereof. Polypeptides, and fragments, derivatives, analogs, or variants thereof of the present invention can be antigenic and immunogenic polypeptides.

The terms “fragment,” “variant,” “derivative,” and “analog,” when referring polypeptides of the present invention, include any polypeptides which retain at least some of the immunogenicity or antigenicity of the corresponding native polypeptide. Fragments of polypeptides of the present invention include proteolytic fragments, deletion fragments, and in particular, fragments of polypeptides which exhibit increased secretion from the cell or higher immunogenicity or reduced pathogenicity when delivered to an animal. Polypeptide fragments further include any portion of the polypeptide which comprises an antigenic or immunogenic epitope of the native polypeptide, including linear as well as three-dimensional epitopes. Variants of polypeptides of the present invention include fragments as described above, and also polypeptides with altered amino acid sequences due to amino acid substitutions, deletions, or insertions. Variants may occur naturally, such as an allelic variant. By an “allelic variant” is intended alternate forms of a gene occupying a given locus on a chromosome or genome of an organism or virus. Genes II, Lewin, B., ed., John Wiley & Sons, New York (1985), which is incorporated herein by reference. Naturally or non-naturally occurring variations such as amino acid deletions, insertions or substitutions may occur. Non-naturally occurring variants may be produced using art-known mutagenesis techniques. Variant polypeptides may comprise conservative or non-conservative amino acid substitutions, deletions or additions. Derivatives of polypeptides of the present invention, are polypeptides which have been altered so as to exhibit additional features not found on the native polypeptide. Examples include fusion proteins. An analog is another form of a polypeptide of the present invention. An example is a proprotein which can be activated by cleavage of the proprotein to produce an active mature polypeptide.

As used herein, an “antigenic polypeptide” or an “immunogenic polypeptide” is a polypeptide which, when introduced into a vertebrate, reacts with the vertebrate's immune system molecules, i.e., is antigenic, and/or induces an immune response in the vertebrate, i.e., is immunogenic. It is quite likely that an immunogenic polypeptide will also be antigenic, but an antigenic polypeptide, because of its size or conformation, may not necessarily be immunogenic.

As used herein, the terms “manufacture,” “produce” or “producing” are defined as making or yielding products or a product. For example, it refers to the manufacture or creation of a desired pharmaceutical composition by methods described herein, whether for commercial use or research purposes.

The term “amphiphilic component” as used herein relates to a molecule having a polar, hydrophilic group attached to a nonpolar, hydrophobic group. Non-limiting examples of hydrophilic groups include groups having a formal charge. Hydrophobic groups include, but are not limited to, groups comprising a substantial hydrocarbon chain. “An amphiphilic component” as used herein can comprise a cationic, anionic or neutral amphiphile or any combination thereof. The amphiphilic component may also comprise other hydrophobic molecules which may be combined with the cationic, anionic or neutral amphiphiles.

As used herein, “mixture” and “solution” are interchangeable.

As used herein, the words “particle” and “microparticle” are interchangeable.

The term “cell delivery particle” as used herein relates to particles comprising an amphiphilic component, a block copolymer, or both, where the particles are stable in aqueous solution, and where the particles can enter cells or provide for the delivery of a pharmaceutical component into cells. Cell delivery particles may facilitate, enhance, or improve entry of a pharmaceutical component into cells, may enhance the potency or efficacy of a pharmaceutical component following its entry into cells or fusion with the cell membrane, e.g., enhance immunogenicity of a pharmaceutical component or an antigen encoded by a pharmaceutical component, improve expression of a polypeptide encoded by a polynucleotide pharmaceutical component, or facilitate proper cell localization of a pharmaceutical component, and may possess one or more of these properties.

As used herein, the term “cloud point” refers to the point in a temperature shift, or other titration, at which a clear solution becomes cloudy, i.e., when a component dissolved in a solution begins to precipitate out of solution.

The term “homogenization,” “homogenizing” or “homogenize” as used herein describes a process by which components of a solution are reduced to particles and dispersed throughout a fluid. “Homogenate” as used herein is the solution after undergoing the homogenization process.

The term “pharmaceutical component-particle dispersion” is intended to encompass cell delivery particles which have been mixed with an additional pharmaceutical component (e.g. a pharmaceutically active drug, an antigenic molecule or a polynucleotide) and are dispersed throughout an aqueous solution.

The term “polydispersity” as used herein is a ratio which represents the molecular weight distribution in a given polymer containing sample. More specifically, polydispersity is the ratio of the number average molecular weight (Mn) to the weight average molecular weight (Mw). If the polydispersity is equal to 1, then Mn equals Mw and the polymer is said to be monodisperse.

As used herein a “block copolymer” is an essentially linear copolymer with chains composed of shorter homo-polymeric chains which are linked together. As used herein the term “block copolymer” and “poloxamer” may be used interchangeably.

The term “stable” as used herein denotes a material which does not readily decompose or undergo a spontaneous change of physical properties.

The methods of the present invention, in one embodiment, relate to a method for manufacturing a cell delivery particle comprising homogenizing, in an aqueous solution, a mixture comprising an amphiphilic component and a block copolymer to form particles. The process of homogenization results in the production of particles containing both block copolymer and amphiphilic components. The homogenization may occur through a variety of means and the mixing of the block copolymer and amphiphilic component may occur simultaneous to homogenization or prior to homogenization.

Homogenization is achieved through a variety of mechanisms and using a variety of devices known in the art including, but not limited to, sonication, high speed blade mixer, a chemical blender, a rotor stator device such as a Silverson mixer (Silverson, United Kingdom) or high pressure homogenizer such as a probe sonicator, a Manton-Gaulin Homogenizer (APV, Albertslund, Denmark), a Sonolator (Sonic Corporation, Stratford, CT), Microfluidizer™ (Microfluidics Corporation, Newton, Mass.), or an EmulsiFlex Homogenizer (Avestin, Ontario, Canada). In a preferred embodiment, an EmulsiFlex high pressure homogenizer is used for homogenization.

The pressure at which the homogenization is performed can range from about 5,000 psi to about 50,000 psi, depending upon the components of the mixture. In a preferred embodiment, the homogenization is performed at a pressure of about 5,000 psi, about 10,000 psi, about 15,000 psi, about 20,000 psi, about 25,000 psi or about 30,000 psi.

Homogenization may be performed at a temperature of about 10° C. to about 100° C., depending upon the components of the mixture. In a preferred embodiment, homogenization is performed at a temperature of about 2° C., about 5° C., about 10° C., about 15° C., about 20° C., about 25° C., about 35° C., about 40° C., about 45° C., about 50° C., about 60° C., about 70° C., about 80° C., about 90° C., about 10° C. and about 110° C. One of ordinary skill in the art will understand that varying temperatures and pressures, as well as varying the components, will affect particle size and stability of cell delivery particles. These conditions can be routinely varied and tested by the methods described herein.

The block copolymers which are useful in the methods and compositions of the present invention are block copolymers which form particles at room temperature. A suitable group of copolymers for use in the present invention include, but are not limited to, non-ionic block copolymers which comprise blocks of polyethylene (POE) and polyoxypropylene (POP), especially higher weight POE-POP-POE block copolymers. These compounds are described in U.S. Reissue Pat. No. 36,665, U.S. Pat. No. 5,567,859, U.S. Pat. No. 5,691,387, U.S. Pat. No. 5,696,298 and U.S. Pat. No. 5,990,241, and WO 96/04392, all of which are hereby incorporated by reference.

Briefly, these non-ionic block copolymers have the following general formula: HO(C₂H₄O)ₓ(C₃H₆O)_{y}(C₂H₄O)ₓH wherein (y) represents a number such that the molecular weight of the hydrophobic POP portion (C₃H₆O) is up to approximately 20,000 daltons and wherein (x) represents a number such that the percentage of hydrophilic POE portion (C₂H₄O) is between approximately 1% and 50% by weight.

A suitable POE-POP-POE block copolymer that can be used in the present invention has the following formula HO(C₂H₄O)ₓ(C₃H₆O)_{y}(C₂H₄O)ₓH wherein (y) represents a number such that the molecular weight of the hydrophobe (C₃H₆O) is between approximately 9000 Daltons and 15,000 Daltons and (x) represents a number such that the percentage of hydrophile (C₂H₄O) is between approximately 3% and 35%.

An alternative POE-POP-POE block copolymer that can be used in the present invention has the following formula: HO(C₂H₄O)ₓ(C₃H₆O)_{y}(C₂H₄O)ₓH wherein (y) represents a number such that the molecular weight of the hydrophobe (C₃H₆O) is between approximately 9000 Daltons and 15,000 Daltons and (x) represents a number such that the percentage of hydrophile (C₂H₄O) is between approximately 3% and 10%.

Yet another suitable block copolymer that can be used in the present invention has the following formula: HO(C₂H₄O)ₓ(C₃H₆O)_{y}(C₂H₄O)ₓH wherein (y) represents a number such that the molecular weight of the hydrophobe (C₃H₆O) is approximately 9000 Daltons and (x) represents a number such that the percentage of hydrophile (C₂H₄O) is approximately 3-5%.

Another alternative block copolymer that can be used in the present invention has the following formula: HO(C₂H₄O)ₓ(C₃H₆O)_{y}(C₂H₄O)ₓH wherein (y) represents a number such that the molecular weight of the hydrophobe (C₃H₆O) is approximately 9000 Daltons and (x) represents a number such that the percentage of hydrophile (C₂H₄O) is approximately 3%.

A suitable block copolymer that can be used in the present invention is CRL-1005. CRL-1005 has the following formula: HO(C₂H₄O)ₓ(C₃H₆O)_{y}(C₂H₄O)ₓH wherein (y) represents a number such that the molecular weight of the hydrophobe (C₃H₆O) is approximately 12000 Daltons and (x) represents a number such that the percentage of hydrophile (C₂H₄O) is approximately 5%, wherein (x) is about 7, ±1 and (y) is about 207 units, ±7.

A suitable block copolymer that can be used in the present invention is CRL-8300. CRL-8300 has the following formula: HO(C₂H₄O)ₓ(C₃H₆O)_{y}(C₂H₄O)ₓH wherein (y) represents a number such that the molecular weight of the hydrophobe (C₃H₆O) is approximately 11000 Daltons and (x) represents a number such that the percentage of hydrophile (C₂H₄O) is approximately 5%, wherein (x) is about 6, +1 and (y) is about 190 units, ±6.

A typical POE/POP block copolymer utilized herein will comprise the structure of POE-POP-POE, as reviewed in Newman et al. (Critical Reviews in Therapeutic Drug Carrier Systems 15 (2): 89-142 (1998)). A suitable block copolymer for use in the methods of the present invention is a POE-POP-POE block copolymer with a central POP block having a molecular weight in a range from about 1000 daltons up to approximately 20,000 daltons and flanking POE blocks which comprise up to about 50% of the total molecular weight of the copolymer. Block copolymers such as these, which are much larger than earlier disclosed Pluronic-based POE/POP block copolymers, are described in detail in U.S. Reissue Pat. No. 36,655. A representative POE-POP-POE block copolymer utilized to exemplify compositions of the present invention is disclosed in Published International Patent Application No. WO 96/04392, is also described at length in Newman et al. (Id.), and is referred to as CRL-1005 (CytRx Corp).

Another suitable group of block copolymers for use in the present invention are “reverse” block copolymers wherein the hydrophobic portions of the molecule (C₃H₆O) and the hydrophilic portions (C₂H₄O) have been reversed such that the polymer has the formula: HO(C₃H₆O)_{y}(C₂H₄O)ₓ(C₃H₆O)_{y}H wherein (y) represents a number such that the molecular weight of the hydrophobic POP portion (C₃H₆O) is up to approximately 20,000 daltons and wherein (x) represents a number such that the percentage of hydrophilic POE portion (C₂H₄O) is between approximately 1% and 50% by weight. These “reverse” block copolymers have the structure POP-POE-POP and are described in U.S. Pat. Nos. 5,656,611 and 6,359,054.

A suitable POP- POE-POP block copolymer that can be used in the invention has the following formula: HO(C₃H₆O)_{y}(C₂H₄O)ₓ(C₃H₆O)_{y}H wherein (y) represents a number such that the molecular weight of the hydrophobe (C₃H₆O) is between approximately 9000 Daltons and 15,000 Daltons and (x) represents a number such that the percentage of hydrophile (C₂H₄O) is between approximately 1% and 95%.

A suitable POP-POE-POP block copolymer that can be used in the invention has the following formula: HO(C₃H₆O)_{y}(C₂H₄O)ₓ(C₃H₆O)_{y}H wherein (y) represents a number such that the molecular weight of the hydrophobe (C₃H₆O) is between approximately 9000 Daltons and 15,000 Daltons and (x) represents a number such that the percentage of hydrophile (C₂H₄O) is between approximately 3% and 35%.

Another suitable POP-POE-POP block copolymer that can be used in the invention has the following formula: HO(C₃H₆O)_{y}(C₂H₄O)ₓ(C₃H₆O)_{y}H wherein (y) represents a number such that the molecular weight of the hydrophobe (C₃H₆O) is between approximately 9000 Daltons and 15,000 Daltons and (x) represents a number such that the percentage of hydrophile (C₂H₄O) is between approximately 3% and 10%.

Another suitable surface-active copolymer that can be used in the invention and has the following formula: HO(C₃H₆O)_{y}(C₂H₄O)ₓ(C₃H₆O)_{y}H wherein (y) represents a number such that the molecular weight of the hydrophobe (C₃H₆O) is approximately 12000 Daltons and (x) represents a number such that the percentage of hydrophile (C₂H₄O) is approximately 5%.

An alternative surface-active copolymer that can be used in the invention has the following formula: HO(C₃H₆O)_{y}(C₂H₄O)ₓ(C₃H₆O)_{y}H wherein (y) represents a number such that the molecular weight of the hydrophobe (C₃H₆O) is approximately 9000 Daltons and (x) represents a number such that the percentage of hydrophile (C₂H₄O) is approximately 3-5%.

Another suitable surface-active copolymer that can be used in the invention has the following formula: HO(C₃H₆O)_{y}(C₂H₄O)ₓ(C₃H₆O)_{y}H wherein (y) represents a number such that the molecular weight of the hydrophobe (C₃H₆O) is approximately 9000 Daltons and (x) represents a number such that the percentage of hydrophile (C₂H₄O) is approximately 3%.

Commercially available block copolymers or poloxamers which can be used in the present invention include, but are not limited to, Pluronic® surfactants, which are block copolymers of propylene oxide and ethylene oxide in which the propylene oxide block is sandwiched between two ethylene oxide blocks. Examples of Pluronic® surfactants include Pluronic® L121 (ave. MW: 4400; approx. MW of hydrophobe, 3600; approx. wt. % of hydrophile, 10%), Pluronic® L101 (ave. MW: 3800; approx. MW of hydrophobe, 3000; approx. wt. % of hydrophile, 10%), Pluronic® L81 (ave. MW: 2750; approx. MW of hydrophobe, 2400; approx. wt. % of hydrophile, 10%), Pluronic® L61 (ave. MW: 2000; approx. MW of hydrophobe, 1800; approx. wt. % of hydrophile, 10%), Pluronic® L31 (ave. MW: 1100; approx. MW of hydrophobe, 900; approx. wt. % of hydrophile, 10%), Pluronic® L122 (ave. MW: 5000; approx. MW of hydrophobe, 3600; approx. wt. % of hydrophile, 20%), Pluronic® L92 (ave. MW: 3650; approx. MW of hydrophobe, 2700; approx. wt. % of hydrophile, 20%), Pluronic® L72 (ave. MW: 2750; approx. MW of hydrophobe, 2100; approx. wt. % of hydrophile, 20%), Pluronic® L62 (ave. MW: 2500; approx. MW of hydrophobe, 1800; approx. wt. % of hydrophile, 20%), Pluronic® L42 (ave. MW: 1630; approx. MW of hydrophobe, 1200;_approx. wt. % of hydrophile, 20%), Pluronic® L63 (ave. MW: 2650; approx. MW of hydrophobe, 1800; approx. wt. % of hydrophile, 30%), Pluronic® L43 (ave. MW: 1850; approx. MW of hydrophobe, 1200; approx. wt. % of hydrophile, 30%), Pluronic® L64 (ave. MW: 2900; approx. MW of hydrophobe, 1800; approx. wt. % of hydrophile, 40%), Pluronic® L44 (ave. MW: 2200; approx. MW of hydrophobe, 1200; approx. wt. % of hydrophile, 40%), Pluronic® L35 (ave. MW: 1900; approx. MW of hydrophobe, 900; approx. wt. % of hydrophile, 50%), Pluronic® P123 (ave. MW: 5750; approx. MW of hydrophobe, 3600; approx. wt. % of hydrophile, 30%), Pluronic® P103 (ave. MW: 4950; approx. MW of hydrophobe, 3000; approx. wt. % of hydrophile, 30%), Pluronic® P104 (ave. MW: 5900; approx. MW of hydrophobe, 3000; approx. wt. % of hydrophile, 40%), Pluronic® P84 (ave. MW: 4200; approx. MW of hydrophobe, 2400; approx. wt. % of hydrophile, 40%), Pluronic® P105 (ave. MW: 6500; approx. MW of hydrophobe, 3000; approx. wt. % of hydrophile, 50%), Pluronic® P85 (ave. MW: 4600; approx. MW of hydrophobe, 2400; approx. wt. % of hydrophile, 50%), Pluronic® P75 (ave. MW: 4150; approx. MW of hydrophobe, 2100; approx. wt. % of hydrophile, 50%), Pluronic® P65 (ave. MW: 3400; approx. MW of hydrophobe, 1800; approx. wt. % of hydrophile, 50%), Pluronic® F127 (ave. MW: 12600; approx. MW of hydrophobe, 3600; approx. wt. % of hydrophile, 70%), Pluronic® F98 (ave. MW: 13000; approx. MW of hydrophobe, 2700; approx. wt. % of hydrophile, 80%), Pluronic® F87 (ave. MW: 7700; approx. MW of hydrophobe, 2400; approx. wt. % of hydrophile, 70%), Pluronic® F77 (ave. MW: 6600; approx. MW of hydrophobe, 2160; approx. wt. % of hydrophile, 70%), Pluronic® F108 (ave. MW: 14600; approx. MW of hydrophobe, 3000; approx. wt. % of hydrophile, 80%), Pluronic® F98 (ave. MW: 13000; approx. MW of hydrophobe, 2700; approx. wt. % of hydrophile, 80%), Pluronic® F88 (ave. MW: 11400; approx. MW of hydrophobe, 2400; approx. wt. % of hydrophile, 80%), Pluronic® F68 (ave. MW: 8400; approx. MW of hydrophobe, 1800; approx. wt. % of hydrophile, 80%), Pluronic® F38 (ave. MW: 4700; approx. MW of hydrophobe, 900; approx. wt. % of hydrophile, 80%).

Commercially available reverse poloxamers which may be used in present invention include, but are not limited to, Pluronic® R 31R1 (ave. MW: 3250; approx. MW of hydrophobe, 3100; approx. wt. % of hydrophile, 10%), Pluronic® R 25R1 (ave. MW: 2700; approx. MW of hydrophobe, 2500; approx. wt. % of hydrophile, 10%), Pluronic® R 17R1 (ave. MW: 1900; approx. MW of hydrophobe, 1700; approx. wt. % of hydrophile, 10%), Pluronic® R 31R2 (ave. MW: 3300; approx. MW of hydrophobe, 3100; approx. wt. % of hydrophile, 20%), Pluronic® R 25R2 (ave. MW: 3100; approx. MW of hydrophobe, 2500; approx. wt. % of hydrophile, 20%), Pluronic® R 17R2 (ave. MW: 2150; approx. MW of hydrophobe, 1700; approx. wt. % of hydrophile, 20%), Pluronic® R 12R3 (ave. MW: 1800; approx. MW of hydrophobe, 1200; approx. wt. % of hydrophile, 30%), Pluronic® R 31R4 (ave. MW: 4150; approx. MW of hydrophobe, 3100; approx. wt. % of hydrophile, 40%), Pluronic® R 25R4 (ave. MW: 3600; approx. MW of hydrophobe, 2500; approx. wt. % of hydrophile, 40%), Pluronic® R 22R4 (ave. MW: 3350; approx. MW of hydrophobe, 2200; approx. wt. % of hydrophile, 40%), Pluronic® R 17R4 (ave. MW: 3650; approx. MW of hydrophobe, 1700; approx. wt. % of hydrophile, 40%), Pluronic® R 25R5 (ave. MW: 4320; approx. MW of hydrophobe, 2500; approx. wt. % of hydrophile, 50%), Pluronic® R 10R5 (ave. MW: 1950; approx. MW of hydrophobe, 1000; approx. wt. % of hydrophile, 50%), Pluronic® R 25R8 (ave. MW: 8550; approx. MW of hydrophobe, 2500; approx. wt. % of hydrophile, 80%), Pluronic® R 17R8 (ave. MW: 7000; approx. MW of hydrophobe, 1700; approx. wt. % of hydrophile, 80%), and Pluronic® R 10R8 (ave. MW: 4550; approx. MW of hydrophobe, 1000; approx. wt. % of hydrophile, 80%).

Other commercially available poloxamers which may used according to the present invention include compounds that are block copolymer of polyethylene and polypropylene glycol such as Synperonic® L121 (ave. MW: 4400), Synperonic® L122 (ave. MW: 5000), Synperonic® P104 (ave. MW: 5850), Synperonic® P105 (ave. MW: 6500), Synperonic® P123 (ave. MW: 5750), Synperonic® P85 (ave. MW: 4600) and Synperonic® P94 (ave. MW: 4600), in which L indicates that the surfactants are liquids, P that they are pastes, the first digit is a measure of the molecular weight of the polypropylene portion of the surfactant and the last digit of the number, multiplied by 10, gives the percent ethylene oxide content of the surfactant; and compounds that are nonylphenyl polyethylene glycol such as Synperonic® NP10 (nonylphenol ethoxylated surfactant—10% solution), Synperonic® NP30 (condensate of 1 mole of nonylphenol with 30 moles of ethylene oxide) and Synperonic® NP5 (condensate of 1 mole of nonylphenol with 5.5 moles of naphthalene oxide).

Additional poloxamers which may be used according to the present invention include: (a) a polyether block copolymer comprising an A-type segment and a B-type segment, wherein the A-type segment comprises a linear polymeric segment of relatively hydrophilic character, the repeating units of which contribute an average Hansch-Leo fragmental constant of about −0.4 or less and have molecular weight contributions between about 30 and about 500, wherein the B-type segment comprises a linear polymeric segment of relatively hydrophobic character, the repeating units of which contribute an average Hansch-Leo fragmental constant of about −0.4 or more and have molecular weight contributions between about 30 and about 500, wherein at least about 80% of the linkages joining the repeating units for each of the polymeric segments comprise an ether linkage; (b) a block copolymer having a polyether segment and a polycation segment, wherein the polyether segment comprises at least an A-type block, and the polycation segment comprises a plurality of cationic repeating units; and (c) a polyether-polycation copolymer comprising a polymer, a polyether segment and a polycationic segment comprising a plurality of cationic repeating units of formula —NH—R^{o}, wherein R^{o} is a straight chain aliphatic group of 2 to 6 carbon atoms, which may be substituted, wherein said polyether segments comprise at least one of an A-type of B-type segment. See U.S. Pat. No. 5,656,611, by Kabonov, et al., which is incorporated herein by reference in its entirety. Other poloxamers of interest include CRL-1005 (12 kDa, 5% POE), CRL-8300 (11 kDa, 5% POE), CRL-2690 (12 kDa, 10% POE), CRL-4505 (15 kDa, 5% POE) and CRL-1415 (9 kDa, 10% POE).

The poloxamer CRL-2690 has the following formula: HO(C₂H₄O)ₓ(C₃H₆O)_{y}(C₂H₄O)ₓH wherein (y) represents a number such that the molecular weight of the hydrophobe (C₃H₆O) is approximately 12000 Daltons and (x) represents a number such that the percentage of hydrophile (C₂H₄O) is approximately 5%, wherein (x) is about 14, ±2 and (y) is about 207 units, ±7.

The poloxamer CRL-4505 has the following formula: HO(C₂H₄O)ₓ(C₃H₆O)_{y}(C₂H₄O)ₓH wherein (y) represents a number such that the molecular weight of the hydrophobe (C₃H₆O) is approximately 15000 Daltons and (x) represents a number such that the percentage of hydrophile (C₂H₄O) is approximately 5%, wherein (x) is about 9, ±1 and (y) is about 259 units, ±7.

The poloxamer CRL-1415 has the following formula: HO(C₂H₄O)ₓ(C₃H₆O)_{y}(C₂H₄O)ₓH wherein (y) represents a number such that the molecular weight of the hydrophobe (C₃H₆O) is approximately 9000 Daltons and (x) represents a number such that the percentage of hydrophile (C₂H₄O) is approximately 10%, wherein (x) is about 21, ±2 and (y) is about 155 units, ±6.

In a preferred embodiment the block copolymer used in the methods and compositions of the present invention is CRL-1005 or CRL-8300.

The concentration of block copolymer used in the invention is adjusted depending on, for example, transfection efficiency, expression efficiency, or immunogenicity. In certain embodiments, the final concentration of the block copolymer is between about 0.1 mg/mL to about 75 mg/mL, for example, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 2 mg/mL, about 3 mg/mL, about 4 mg/mL, about 5 mg/mL, about 3 mg/mL to about 50 mg/mL, about 6 mg/mL, about 6.5 mg/mL, about 7 mg/mL, about 7.5 mg/mL, about 8 mg/mL, about 9 mg/mL, about 10 mg/mL, about 15 mg/mL, about 20 mg/mL, about 25 mg/mL, about 30 mg/mL, about 35 mg/mL, about 40 mg/mL, about 50 mg/mL, about 60 mg/mL, about 70 mg/mL, or about 75 mg/mL of block copolymer.

The amphiphilic component for use in the methods and compositions of the present invention may comprise any amphiphile including a cationic amphiphile, an anionic amphiphile, a neutral amphiphile or any combination thereof.

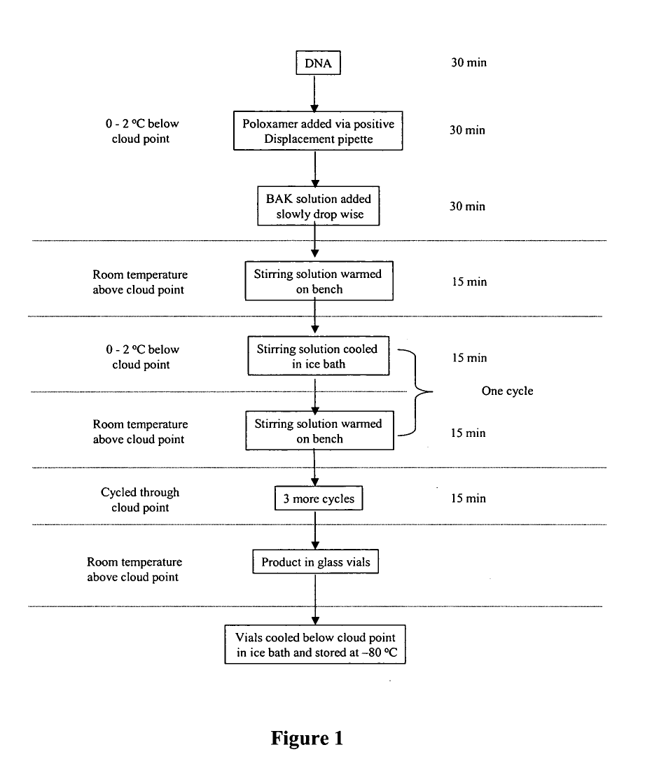

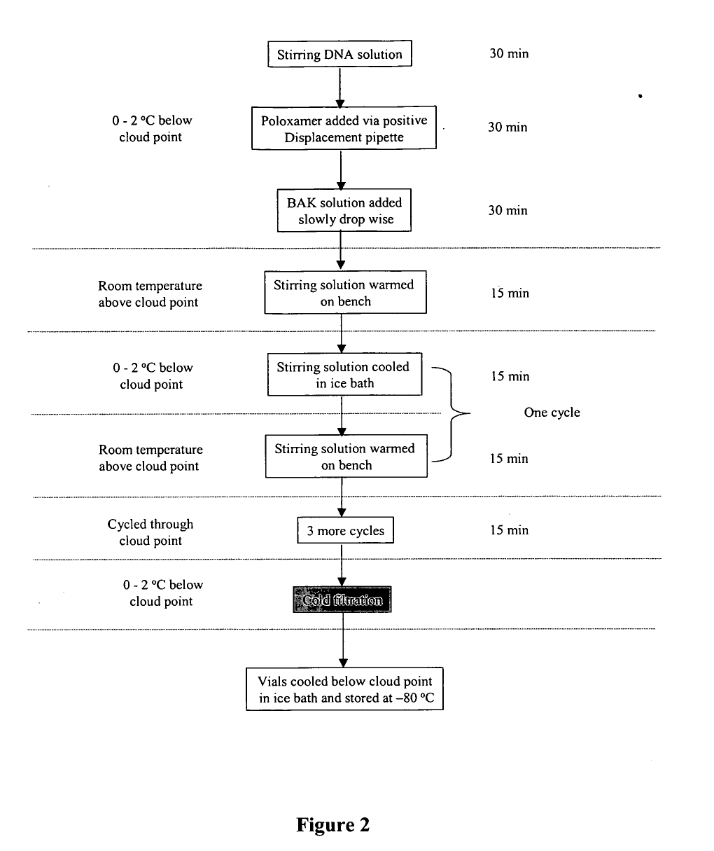

Amphiphilic components suitable for use in the present invention are lipids which are not soluble in an aqueous solution below about 5° C. These lipids usually have longer alkyl chains which result in reduced solubility at lower temperatures. Lipid/block copolymer combinations, in which the lipid is not soluble around the cloud point of the block copolymer, would not routinely succeed in generating homogenous stable particles using previously described thermal cycling methods which require cycling below the cloud point of the block copolymer and are exemplified in FIGS. 1 and 2. These combinations of lipids and block copolymers may be formed using the methods described herein. Thus, certain lipids which are not soluble at temperatures around or below the cloud point of certain block copolymers (e.g. below about 5° C.) are suitable for use in the claimed methods.

In certain embodiments, the amphiphilic component comprises a cationic amphiphile. According to these embodiments, the invention contemplates use of any cationic amphiphile. Cationic amphiphiles which can be used in the present invention include, but are not limited to, benzalkonium chloride (BAK), benzethonium chloride, cetramide (which contains tetradecyltrimethylammonium bromide and possibly small amounts of dedecyltrimethylammonium bromide and hexadecyltrimethyl ammonium bromide), cetylpyridinium chloride (CPC) and cetyl trimethylammonium chloride (CTAC), primary amines, secondary amines, tertiary amines, including but not limited to N,N′,N′-polyoxyethylene (10)-N-tallow-1,3 -diaminopropane, other quaternary amine salts, including but not limited to dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemethanaminium chloride (DEBDA), dialkyldimetylammonium salts, -[1-(2,3-dioleyloxy)-propyl]-N,N,N, trimethylammonium chloride, 1, 2-diacyl-3-(trimethylammonio) propane (acyl group=dimyristoyl, dipalmitoyl, distearoyl dioleoyl), 1,2-diacyl-3 (dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio) butanoate), N-alkyl pyridinium salts (e.g. cetylpyridinium bromide and cetylpyridinium chloride), N-alkylpiperidinium salts, dicationic bolaform electrolytes (C₁₂Me₆; C₁₂Bu₆), dialkylglycetylphosphorylcholine, lysolecithin, L-a dioleoyl phosphatidylethanolamine), cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly (L (or D)-lysine conjugated to N-glutarylphosphatidylethanolamine, didodecyl glutamate ester with pendant amino group (Cl₂GluPhCnN⁺), ditetradecyl glutamate ester with pendant amino group (Cl₄GluCnN⁺), cationic derivatives of cholesterol, including but not limited to cholesteryl-3β-oxysuccinamidoethylenetrimethylammonium salt, cholesteryl-3β-oxysuccinamidoethylenedimethylamine, cholesteryl-3β-carboxyamidoethylenetrimethylammonium salt, cholesteryl-3β-carboxyamidoethylenedimethylamine, and 3γ-[N-(N′,N′-dimethylaminoetanecarbomoyl]cholesterol) (DC-Chol).

Other examples of cationic amphiphiles for use in the invention are selected from the group of cationic lipids including N-(3-aminopropyl)-N,N-(bis-(2-tetradecyloxyethyl))-N-methyl-ammonium bromide (PA-DEMO), N-(3-aminopropyl)-N,N-(bis-(2-dodecyloxyethyl))-N-methyl-ammonium bromide (PA-DELO), N,N,N-tris-(2-dodecyloxy)ethyl-N-(3-amino)propyl-ammonium bromide (PA-TELO), and N¹-(3-aminopropyl)((2-dodecyloxy)ethyl)-N²-(2-dodecyloxy)ethyl-1-piperazin aminium bromide (GA-LOE-BP), DL-1,2-dioleoyl-3-dimethylaminopropyl-β-hydroxyethylammonium (DORI diester), 1-O-oleyl-2-oleoyl-3-dimethylaminopropyl-β-hydroxyethylammonium (DORI ester/ether).

Additional specific, but non-limiting cationic amphiphiles for use in certain embodiments of the present invention include DMRIE ((±)-N-(2-hydroxyethyl)-N,N-dimethyl-2,3-bis(tetradecyloxy)-1-propanaminium bromide), GAP-DMORIE ((±)-N-(3-aminopropyl)-N,N-dimethyl-2,3-bis (syn-9-tetradeceneyloxy)-1-propanaminium bromide), and GAP-DLRIE ((i)-N-(3-aminopropyl)-N,N-dimethyl-2,3-bis-(dodecyloxy)-1-propanaminim bromide).

Other cationic amphiphiles for use in the present invention include the compounds described in U.S. Pat. Nos. 5,264,618, 5,459,127 and 5,994,317. Non-limiting examples of these cationic lipids include (±)-N,N-dimethyl-N-[2-(sperminecarboxamido)ethyl]-2,3-bis(dioleyloxy)-1-propaniminium pentahydrochloride (DOSPA), (±)-N-(2-aminoethyl)-N,N-dimethyl-2,3-bis(tetradecyloxy)-1-propaniminium bromide (β-aminoethyl-DMRIE or βAE-DMRIE), and (i)-N-(3-aminopropyl)-N,N-dimethyl-2,3-bis (dodecyloxy)-1-propaniminium bromide (GAP-DLRIE).

Other examples of DMRIE-derived cationic amphiphiles that are useful for the present invention are (±)-N-(3-aminopropyl)-N,N-dimethyl-2,3-(bis-decyloxy)-1-propanaminium bromide (GAP-DDRIE), (±)-N-(4-aminobutyl)-N,N-dimethyl-2,3-(bis-decyloxy)-1-propanaminium bromide (DAB-DDRIE), (±)-N-(3-aminopropyl)-N,N-dimethyl-2,3-(bis-tetradecyloxy)-1-propanaminium bromide (GAP-DMRIE), (i)-N-((N″-methyl)-N′-ureyl)propyl-N,N-dimethyl-2,3-bis(tetradecyloxy)-1-propanaminium bromide (GMU-DMRIE), (±)-N-(2-hydroxyethyl)-N,N-dimethyl-2,3-bis(dodecyloxy)-1-propanaminium bromide (DLRIE), and (±)-N-(2-hydroxyethyl)-N,N-dimethyl-2,3-bis-([Z]-9-octadecenyloxy)propyl-1-propaniminium bromide (HP-DORIE).

In certain embodiments of the present invention, the cationic amphiphile is selected from the group consisting of benzalkonium chloride, benzethonium chloride, cetramide, cetylpyridinium chloride, cetyl trimethylammonium chloride and the cationic lipid component of Vaxfectin™, (±)-N-(3-aminopropyl)-N,N-dimethyl-2,3-bis(syn-9-tetradeceneyloxy)-1-propanaminium bromide (VC1052). Benzalkonium chloride (BAK) is available commercially and is known to exist as a mixture of alkylbenzyldimethylammonium chlorides of the general formula: [C₆H₅CH₂N(CH₃) 2R]Cl, where R represents a mixture of alkyl chains, including all or some of the group beginning with n-C₈H₁₇ through n-C₁₈H₃₃. The average MW of BAK is 360. Wade and Weller, Handbook of Pharmaceutical Excipients 27-29 (2nd ed. 1994). See also Susan Budavari, Merck Index 177 (12th ed. 1996). Benzethonium chloride is N, N-dimethyl-N-[2-[2-[4-(1,1,3,3 tetramethylbutyl)phenoxy]ethoxy]ethyl]benzene-methanaminium chloride (C₂₇H₄₂ClNO₂), which has a molecular weight of 448.10 (Handbook of Pharmaceutical Excipients at page 30-31). Cetramide consists mainly of trimethyltetradecylammonium bromide (C₁₇H₃₈BrN), which may contain smaller amounts of dodecyltrimethyl-ammonium bromide (C₁₅H₃₄BrN) and hexadecyltrimethylammonium bromide (C₁₉H₄₂BrN), and has a molecular weight of 336.40 (Handbook of Pharmaceutical Excipients at page 96-98).

In particular embodiments, the benzalkonium chloride comprises more than about 90% of a particular alkyl chain isomer such as C₁₂, C₁₄, C₁₆ or C₁₈. In other embodiments the benzalkonium chloride comprise more than 95% of a particular alkyl chain isomer such as C₁₂, C₁₄, C₁₆ or C₁₈.

Examples of additional useful cationic amphiphiles of the present invention include: (±)-N-(Benzyl)-N,N-dimethyl-2,3-bis(hexyloxy)-1-propanaminium bromide (Bn-DHxRIE), (±)-N-(2-Acetoxyethyl)-N,N-dimethyl-2,3-bis(hexyloxy)-1-propanaminium bromide (DHxRIE-OAc), (±)-N-(2-Benzoyloxyethyl)-N,N-dimethyl-2,3-bis(hexyloxy)-1-propanaminium bromide (DHxRIE-OBz), (±)-N-(3-Acetoxypropyl)-N,N-dimethyl-2,3-bis(octyloxy)-1-propanaminium chloride (Pr-DOctRIE-OAc). The structures of these compounds are described in U.S. Patent Application Publication 2004/0162256 A1 and the general structures are described in U.S. Pat. No. 5,264,618 and U.S. Pat. No. 5,459,127, all of which are incorporated herein by reference.

In certain embodiments, the amphiphilic component comprises an anionic amphiphile. Examples of anionic amphiphiles which may be used in the present invention include, but are not limited to, phosphatidyl serine chenodeoxycholic acid sodium salt, dehydrocholic acid sodium salt, deoxycholic acid, docusate sodium salt, glycocholic acid sodium salt, glycolithocholic acid 3-sulfate disodium salt, N-lauroylsarcosine sodium salt, lithium dodecyl sulfate, 1-octanesulfonic acid sodium salt, sodium 1-decanesulfonate, sodium 1-dodecanesulfonate, sodium choleate, sodium deoxycholate, sodium dodecyl sulfate, taurochenodeoxycholic acid sodium salt, taurolithocholic acid 3-sulfate disodium salt, 1,2-dimyristoyl-sn-glycero-3-phosphate sodium salt, 1-palmitoyl-2-oleoyl-sn-glycero-3-phosphate sodium salt, 1,2-diphytanoyl-sn-glycero-3-phosphate sodium salt, dodecanoic acid sodium salt and octadecanoic acid sodium salt.

In certain embodiments, the amphiphilic component comprises a zwitterionic amphiphile. Examples of zwitterionic amphiphiles which may be used in the present invention include, but are not limited, to phosphatidylcholine (PC), phosphatidylethanolamine (PE), fully or partially hydrogenated PC or PE, phosphatidylethanolomines having aliphatic chains between 6 and 24 carbons in length such as dioleoyl-PC (DOPC) and dioleoyl-PE (DOPE). In additional embodiments, the amphiphilic component comprises a neutral component such as a neutral detergent (e.g. Tween). Non-limiting examples of neural components include non-ionic surfactants are described elsewhere herein.

The concentration of the amphiphilic component may be adjusted depending on, for example, a desired particle size and improved stability. In general the amphiphilic component of the present invention is adjusted to have a final concentration from about 0.001 mM to about 10 mM. A suitable formulation of the present invention may have a final concentration of amphiphilic component of about 0.001 mM, about 0.005 mM, about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1.0 mM, about 2.0 mM, about 3.0 mM, about 4.0 mM, about 5.0 mM, about 6.0 mM, about 7.0 mM, about 8.0 mM, about 9.0 mM or about 10.0 mM.

Additionally, the concentration of a specific amphiphilic component, such as BAK, may be adjusted depending on, for example, a desired particle size and improved stability. Such adjustments may be routinely carried out and tested using the methods described herein. Indeed, in certain embodiments, the methods of the present invention are adjusted to have a final concentration of BAK from about 0.01 mM to about 5 mM. A suitable composition of the present invention may have a final BAK concentration of about 0.06 mM to about 1.2 mM, or about 0.1 mM to about 1 mM, or about 0.2 mM to about 0.7 mM. For example, a formulation of the present invention may have a final BAK concentration of about 0.05 mM, 0.1 mM, 0.2 mM, 0.3 mM, 0.4 mM, or 0.5 mM or about 0.6 mM, or about 0.7 mM.

The methods of the present invention entail homogenization of the amphiphilic component and block copolymer in any aqueous solution. Aqueous solutions suitable for the present invention include, but are not limited to water, saline, PBS, N-(2-Hydroxyethyl)piperazine-N′-(2-ethanesulfonic acid) (HEPES), 3-(N-Morpholino)propanesulfonic acid (MOPS), 2-bis(2-Hydroxyethyl)amino-2-(hydroxymethyl)-1,3-propanediol (BIS-TRIS), potassium phosphate (KP), sodium phosphate (NaP), dibasic sodium phosphate (Na₂HPO₄), monobasic sodium phosphate (NaH₂PO₄), monobasic sodium potassium phosphate (NaKHPO₄), magnesium phosphate (Mg₃(PO₄)₂.4H₂O), potassium acetate (CH₃COOK), D(+)-α-sodium glycerophosphate (HOCH₂CH(OH)CH₂OPO₃Na₂) and other aqueous solutions known to those skilled in the art.

In certain embodiments, the aqueous solution comprises a physiologic buffer. Physiologic buffers suitable for the present invention maintain the solution pH within the range of about pH 4.0 to about pH 9.0. In certain embodiments, the pH of the homogenate comprising a physiologic buffer is about pH 5.0 to about pH 8.0, about pH 6.0 to about pH 8.0, or about pH 7.0 to about pH 7.5. For example, the pH of the homogenized mixture is about pH 7.0, about pH 7.1, about pH 7.2, about pH 7.3, about pH 7.4 or about pH 7.5

Example of suitable physiological buffers for use in the invention include buffers comprising a salt M-X dissolved in aqueous solution, association, or dissociation products thereof, where M is an alkali metal (e.g., Li⁺, Na⁺, K⁺, Rb⁺), suitably sodium or potassium, and where X is an anion selected from the group consisting of phosphate, acetate, bicarbonate, sulfate, pyruvate, and an organic monophosphate ester, preferably glucose 6-phosphate or DL-α-glycerol phosphate and other physiologic buffers known to those skilled in the art. In a suitable embodiment of the invention, the physiologic buffering agent present in the aqueous solution is selected from the group consisting of a phosphate anion, sodium phosphate, potassium phosphate, dibasic sodium phosphate (Na₂HPO₄), monobasic sodium phosphate (NaH₂PO₄), monobasic sodium potassium phosphate (NaKHPO₄), magnesium phosphate (Mg₃(PO₄)₂.4H₂O), potassium acetate (CH₃COOK), and D(+)-α-sodium glycerophosphate (HOCH₂CH(OH)CH₂OPO₃Na₂).

In a suitable embodiment of the invention, the concentration of the buffering agent or anion is from about 5 mM to about 150 mM. Suitably, compositions of the present invention has a final pH buffering agent or anion concentration of about 5 mM to about 100 mM, 5 mM to about 75 mM, about 5 mM to about 50 mM, about 5 mM to about 25 mM or about 5 mM to about 10 mM. For example, a formulation of the present invention may have a final buffering agent concentration of about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, or about 35 mM. In another suitable embodiment, the concentration of the pH buffering agent is about 10 mM.

In another suitable embodiment of the invention, the concentration of the pH buffering agent selected from the group consisting of a phosphate anion, sodium phosphate, potassium phosphate, Na₂HPO₄, NaH₂PO₄, NaKHPO₄, Mg₃(PO₄)₂.4H₂O, and HOCH₂CH(OH)CH₂OPO₃Na₂ is from about 5 mM to about 25 mM. Suitably, a formulation of the present invention may have a final concentration of pH buffering agent selected from the group consisting of a phosphate anion, sodium phosphate, potassium phosphate, Na₂HPO₄, NaH₂PO₄, NaKHPO₄, Mg₃(PO₄)₂.4H₂O, and HOCH₂CH(OH)CH₂OPO₃Na₂ of about 7 mM to about 20 mM, about 8 mM to about 15 mM, or about 9 mM to about 12 mM. For example, a formulation of the present invention may have a final concentration of pH buffering agent selected from a phosphate anion, sodium phosphate, potassium phosphate, Na₂HPO₄, NaH₂PO₄, NaKHPO₄, Mg₃(PO₄)₂.4H₂O, and HOCH₂CH(OH)CH₂OPO₃Na₂ of about 9 mM, about 10 mM, about 11 mM, or about 12 mM. In another suitable embodiment, the concentration of pH buffering agent selected from a phosphate anion, sodium phosphate, potassium phosphate, Na₂HPO₄, NaH₂PO₄, NaKHPO₄, Mg₃(PO₄)₂.4H₂O, and HOCH₂CH(OH)CH₂OPO₃Na₂ is about 10 mM. In an alternative embodiment, the phosphate anion is present in solution at a concentration of about 10 mM.

Additionally, the aqueous solution may contain additional components such as stabilizer, antibiotics, antifungal or antimycotic agents.

In certain methods of the present invention, cell delivery particles produced by the methods described herein are further mixed with a pharmaceutical component selected from the group consisting of a pharmaceutically active drug, an antigenic molecule and a polynucleotide to form a pharmaceutical component particle dispersion. It is understood, however, that any pharmaceutical component may be used with the cell delivery particles of the present invention. As used herein a “pharmaceutical component” is any ingredient added to the cell delivery particles of the invention which when administered to a vertebrate has a therapeutic, ameliorating or prophylactic effect, e.g, preventing, curing, retarding, or reducing the severity of symptoms, and/or result in no worsening of symptoms, of a specific disease or condition over a specified period of time. Examples of pharmaceutical components are described herein and include polynucleotides, antigenic agents and pharmaceutically active drugs.

Examples of pharmaceutically active drugs which may be used in the methods and pharmaceutical compositions of the present invention, include but are not limited to vitamins, local anesthetics (e.g. procaine), antimalarial agents (e.g. chloroquine), anti-parkinsons agents (e.g. leva-DOPA), adrenergic receptor agonists (e.g. propanolol), antibiotics (e.g. anthracycline), anti-neoplastic agents (e.g. doxorubicin), antihistimines, biogenic amines (e.g.dopamine), antidepressants (e.g. desipramine), anticholergenics (e.g. atropine), antiarrhythnics (e.g. quinidine), antiemimetics (e.g. chloroprimamine), analgesics (e.g. codeine, morphine) and hormones (e.g. estrogen) or small molecular weight drugs such as cisplatin which enhance transfection efficiency or prolong the half life of DNA in an outside cells.

In particular embodiments, the pharmaceutical component is an antigenic molecule. As used herein, an “antigenic molecule” or an “immunogenic molecule” is typically a polypeptide which, when introduced into a vertebrate or expressed by a vertebrate, reacts with the immune system molecules of the vertebrate, i.e., is antigenic, and/or induces an immune response in the vertebrate, i.e., is immunogenic. Pharmaceutical components further include polynucleotides encoding antigenic or immunogenic molecules. Such polynucleotides are described in detail elsewhere herein. It is quite likely that an immunogenic polypeptide will also be antigenic, but an antigenic polypeptide, because of its size or conformation, may not necessarily be immunogenic. Non limiting examples of antigenic molecules which can be used in the methods or compositions of the present invention include haptens, proteins, nucleic acids, tumor cells and antigens from various sources such as infectious agents. Antigenic molecules further include inactivated or attenuated infectious agents, or some part of the infectious agents, live or killed microorganism, or a natural product purified from a microorganism or other cell including, but not limited to tumor cells, a synthetic product, a genetically engineered protein, peptide, polysaccharide or similar product or an allergen. The antigenic molecule can also be a subunit of a protein, peptide, polysaccharide or similar product or a polynucleotide which encodes an antigenic polypeptide, which when present in an effect amount results in a detectable immune response. Antigenic or immunogenic molecules also include, e.g., carbohydrates, nucleic acids and small molecules (e.g. dinitrophenol (DNP)).

Additional pharmaceutical components for the purposes of the present invention include immunoglobulin molecules or antibodies, which specifically bind to an antigenic or immunogenic molecule. Non-limiting examples include: immunoglobulin molecules or fragments thereof, Fab, Fab′, F(ab′)2, Fd, single-chain Fvs, single chain immunoglobulins, disulfide linked Fvs, scFv minibodies, diabodies, triabodies, tetrabodies, Fab minibodies and dimeric scFv and any other fragments comprising a VL and a VH domain in a conformation such that a complementary determining region (CDR) specific for the antigenic or immunogenic molecule of interest is formed.

In certain embodiments, the pharmaceutical component is a polynucleotide. Non-limiting examples include plasmid DNA, genomic DNA, complementary DNA (cDNA), antisense DNA, fragments and RNA. Specific RNA contemplated by the invention include, but are not limited to, messenger RNA (mRNA), antisense RNA, double-stranded RNA (dsRNA), transfer RNA (tRNA), ribosomal RNA (rRNA) and ribozymes and any DNA which would encode for specific RNAs.

RNAs which may be used in the present include, inter alia, exonuclease-resistant RNAs such as circular mRNA, chemically blocked mRNA, short interfering RNA (siRNA), and mRNA with a 5′ cap are preferred. In particular, one preferred mRNA is a self-circularizing mRNA having the gene of interest preceded by the 5′ untranslated region of polio virus. It has been demonstrated that circular mRNA has an extremely long half-life (Harland & Misher, Development 102: 837-852 (1988)) and that the polio virus 5′ untranslated region can promote translation of mRNA without the usual 5′ cap (Pelletier & Sonnenberg, Nature 334:320-325 (1988), hereby incorporated by reference). In addition, the present invention includes the use of mRNA that is chemically blocked at the 5′ and/or 3′ end to prevent access by RNAse. (This enzyme is an exonuclease and therefore does not cleave RNA in the middle of the chain.) Such chemical blockage can substantially lengthen the half life of the RNA in vivo. Two agents which may be used to modify RNA are available from Clonetech Laboratories, Inc., Palo Alto, Calif.: C2 AminoModifier (Catalog # 5204-1) and Amino-7-dUTP (Catalog # K1022-1). These materials add reactive groups to the RNA. After introduction of either of these agents onto an RNA molecule of interest, an appropriate reactive substituent can be linked to the RNA according to the manufacturer's instructions. By adding a group with sufficient bulk, access to the chemically modified RNA by RNAse can be prevented.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

Certain compositions produced by the methods of the present invention include a cocktail of polynucleotides. Various DNAs or RNAs or combinations thereof which are desired in a cocktail, are combined together in PBS or other diluent in addition to the particles of the present invention. There is no upper limit to the number of different types of polynucleotides which can be used in the method of the present invention. Furthermore, polynucleotides may be present in equal proportions, or the ratios may be adjusted based on, for example, relative expression levels, relative immunogenicity of the encoded antigens or relative half-lives of the polynucleotides.

It is understood that if the polynucleotides of the present invention are to be expressed, that the polynucleotides comprise appropriate signals for their transcription or translation. The appropriate signals such as promoters or translational start sites are described supra.

Similarly the concentration of a polynucleotide to be used in the compositions and methods of the current invention is adjusted depending on many factors, including the amount of pharmaceutical composition to be delivered, the age and weight of the subject, the delivery method and route of the polynucleotide being delivered. In a suitable embodiment, the final concentration of polynucleotide is from about 1 ng/mL to about 50 mg/mL of plasmid (or other polynucleotide). For example, certain pharmaceutical component-particle dispersions and pharmaceutical compositions comprising the same have a final concentration of about 0.1 mg/mL to about 20 mg/mL, about 1 mg/mL to about 10 mg/mL, about 1 mg/mL, about 2 mg/mL, about 2.5, about 3 mg/mL, about 3.5, about 4 mg/mL, about 4.5, about 5 mg/mL, about 5.5 mg/mL, about 6 mg/mL, about 7 mg/mL, about 8 mg/mL, about 9 mg/mL, about 10 mg/mL, about 20 mg/mL, about 30 mg/mL, about 40 mg/mL or about 50 mg/mL of a particular polynucleotide. One of ordinary skill in the art can routinely determine an optimal polynucleotide concentration.

Pharmaceutical component-particle dispersions produced by the methods of the present invention may be formulated in any pharmaceutically effective formulation to form a pharmaceutical composition for host administration. Any such formulation may be the aqueous solutions described supra (e.g. a saline solution such as phosphate buffered saline (PBS)). It will be useful to utilize pharmaceutically acceptable formulations which also provide long-term stability of the cell delivery particles or pharmaceutical component-particle dispersions of the present invention. For example, during storage as a pharmaceutical entity, DNA plasmids may undergo a physiochemical change in which the supercoiled plasmid converts to the open circular and linear form. A variety of storage conditions (low pH, high temperature, low ionic strength) can accelerate this process. Therefore, the removal and/or chelation of trace metal ions (with succinic or malic acid, or with chelators containing multiple phosphate ligands, or with chelating agents such as EDTA) from the DNA solution, from the formulation buffers or from the vials and closures, stabilizes the DNA plasmid from this degradation pathway during storage.

In addition, inclusion of non-reducing free radical scavengers, such as ethanol or glycerol, are useful to prevent damage of DNA from free radical production that may still occur, even in apparently demetalated solutions. Therefore, formulations that will provide the highest stability of the pharmaceutical compositions comprising polynucleotides will be one that includes a demetalated solution containing a buffer (bicarbonate) with a pH in the range of 7-8, a salt (NaCl, KCl or LiCl) in the range of 100-200 mM, a metal ion chelator (e.g., EDTA, diethylenetriaminepenta-acetic acid (DTPA), malate, a nonreducing free radical scavenger (e.g., ethanol, glycerol, methionine or dimethyl sulfoxide) and an appropriate polynucleotide concentration in a sterile glass vial, packaged to protect the highly purified, nuclease free polynucleotide from light. A formulation which will enhance long term stability of the polynucleotide based medicaments comprises a Tris-HCl buffer at a pH from about 8.0 to about 9.0; ethanol or glycerol at about 0.5-3% w/v; EDTA or DTPA in a concentration range up to about 5 mM; and NaCl at a concentration from about 50 mM to about 500 mM. The use of stabilized DNA vector-based medicaments and various alternatives to this suitable formulation range is described in detail in PCT International Application No. PCT/US97/06655, Published International Patent Application No. WO 97/40839, which is hereby incorporated by reference.

In certain embodiments, one or more co-lipids is mixed with the amphiphilic component and block copolymer prior to the formation of cell delivery particles. For purposes of definition, the term “co-lipid” refers to any hydrophobic material which may be combined with the amphiphilic component and block copolymer mixture and includes amphipathic lipids, such as phospholipids, and other molecules such as cholesterol. One non-limiting class of co-lipids are the zwitterionic phospholipids, which include the phosphatidylethanolamines and the phosphatidylcholines. Examples of phosphatidylethanolamines, include DOPE, DMPE and DPyPE. In certain embodiments, the co-lipid is DPyPE, which comprises two phytanoyl substituents incorporated into the diacylphosphatidylethanolamine skeleton. In other embodiments, the co-lipid is DOPE, CAS name 1,2-diolyeoyl-sn-glycero-3-phosphoethanolamine.

When cell delivery particles of the present invention comprise an amphiphilic component and a co-lipid, the amphiphilic component:co-lipid molar ratio may be from about 9:1 to about 1:9, from about 4:1 to about 1:4, from about 2:1 to about 1:2, or about 1:1.

Other hydrophobic and amphiphilic additives, such as, for example, sterols, fatty acids, gangliosides, glycolipids, lipopeptides, liposaccharides, neobees, niosomes, prostaglandins and sphingolipids, may also be included in cell delivery particles of the present invention. These additives may be included in an amount between about 0.1 mol % and about 99.9 mol % (relative to total lipid), about 1-50 mol %, or about 2-25 mol %.

The artisan will be able to mix and match various block copolymers, amphiphilic components and additional components described herein, as well as utilize various concentrations of these components to produce cell delivery particles, pharmaceutical component-particle dispersions, cell delivery particle compositions or pharmaceutical compositions comprising same which meet the needs of the artisan.

In further embodiments, the methods of the present invention comprises a lyophilization step. As used herein, “lyophilization” is a means of drying, achieved by rapid dehydration by sublimation under a vacuum level down to the lower level of a diffusion pump. A useful vacuum range is from about 0.1 mTorr to about 0.5 Torr. The term “freeze-drying” may be used interchangeably with the term “lyophilization” herein. The present methods result in cell delivery particles, pharmaceutical component-particle dispersions, cell delivery particle compositions or pharmaceutical compositions comprising block copolymer and amphiphilic components that upon reconstitution maintain substantially the same particle size and polydispersity as the cell delivery particles, pharmaceutical component-particle dispersions, cell delivery particle compositions or pharmaceutical compositions comprising same prior to lyophilization.

The methods of the current invention provide for a method of lyophilizing the homogenate or component-particle dispersions which are produced by the methods of the present invention. Prior to lyophilization, the homogenates are flash frozen at a temperature of about −200° C. to about −150° C. The flash freezing may be performed by any means. A non-limiting example of a flash freezing method is via liquid nitrogen.

After flash freezing, e.g. in liquid nitrogen or any other suitable cold, high heat capacity medium such as a dry ice—ethanol slurry, the frozen homogenate or component-particle dispersions, are subject to lyophilization initially at temperatures ranging from about −80° C. to about −20° C. Specifically, lyophilization may be performed at a temperature including but not limited to −90° C., about −85° C., about −80° C., about −75° C., about −70° C., about −65° C., about −60° C., about −55° C., about −50° C., about 45° C., about −40° C., about −35° C., about −30° C., about −25° C., about −20° C., about −15° C. or any combination thereof.

The claimed methods may optionally include a second drying step performed at a temperature of about 10° C. to about 40° C. Lyophilization may be performed in any suitable lyophilizing apparatus that can hold a pressure of from about 0.1 mTorr to about 0.5 Torr. A non-limiting example of a lyophilizing instrument is a freeze-dryer, specifically a Virtis Advantage freeze-dryer. Lyophilization in the present methods may range from 100 mTorr to about 500 mTorr.

The cell delivery particles, pharmaceutical component-particle dispersions, cell delivery particle compositions and pharmaceutical compositions of the present invention may further comprise a cryoprotectant or amorphous cryoprotectant. As used herein, the term “amorphous cryoprotectant” refers to a compound which, when included in the formulations of the present invention during freezing or lyophilization under given conditions, does not form crystals. It is specifically intended that compounds that are known to form crystals under certain lyophilization conditions, but not under others, are included within the term “amorphous cryoprotectant,” so long as they remain amorphous under the specific freezing or lyophilization conditions to which they are subjected. The term “cryoprotectant” may be used interchangeably with the term “amorphous cryoprotectant” herein. The cryoprotectant may be added to the mixture of components prior to, during or after homogenization to produce the cell delivery particles or pharmaceutical component particle dispersions of the invention.

As used herein, “crystalline bulking agent” refers to a compound which, when included in the cell delivery particles, pharmaceutical component-particle dispersions, cell delivery particle compositions or pharmaceutical compositions of the present invention during freezing or lyophilization under given conditions, forms crystals. It is specifically intended that compounds that are known to form crystals under certain lyophilization conditions but not under others are included within the term “crystalline bulking agent,” so long as they crystallize under the specific freezing or lyophilization conditions to which they are subjected. The term “bulking agent” may be used interchangeably with the term “crystalline bulking agent” herein.

Amorphous cryoprotectants, crystalline bulking agents, and methods of determining the same are known and available in the art and may be routinely selected and tested by one of ordinary skill in the art using the methods described herein. See e.g., articles incorporated herein by reference in their entireties: Osterberg et al., Pharm Res 14(7):892-898 (1997); Oliyai et al., Pharm Res 11(6):901-908 (1994); Corveleyn et al., Pharm Res 13(1):146-150 (1996); Kim et al., J. Pharm Sciences 87(8):931-935 (1998); Martini et al., PDA J. Pharm Sci Tech 51(2):62-67 (1997); Martini et al., STP Pharma Sci. 7(5):377-381 (1997); and Orizio et al., Boll. Chim. Farm. 132(9):368-374 (1993).

Amorphous cryoprotectants which are suitable for use herein include, but are not limited to, mono, di, or oligosaccharides, polyols, and proteins such as albumin; disaccharides such as sucrose and lactose; monosaccharides such as fructose, galactose and glucose; poly alcohols such as glycerol and sorbitol; and hydrophilic polymers such as polyethylene glycol.

The amorphous cryoprotectant is suitably added to the cell delivery particles, pharmaceutical component-particle dispersions, cell delivery particle compositions or pharmaceutical compositions of the present invention before freezing, in which case it can also serve as a bulking agent.

With regard to crystalline bulking agents, such agents are often used in the preparation of pharmaceutical compositions to provide the necessary bulk upon lyophilization. Many types of crystalline bulking agents are known in the art. (See, Martini et al., PDA J. Pharm Sci Tech 51(2):62-67, (1997)). Exemplary crystalline bulking agents include D-mannitol, trehalose, and dextran. As the aforementioned are exemplary only, one skilled in the art would recognize that any compound which, when included in the cell delivery particles, pharmaceutical component-particle dispersions, cell delivery particle compositions or pharmaceutical compositions of the present invention during freezing or lyophilization under given conditions, forms crystals, would be considered a suitable crystalline bulking agent. Within the context of the present invention a crystalline bulking agent is generally defined as a compound which can exist in a crystalline form and whose glass transition point (Tg) is below the temperature at which it is being freeze-dried. For example, a conventional freeze-dryer operates at a shelf-temperature from between about −10° C. to about −50° C. Therefore, in one embodiment, a crystalline bulking agent has a Tg below about −50° C.

In a suitable embodiment, a cell delivery particle, pharmaceutical component-particle dispersion, cell delivery particle composition or pharmaceutical composition comprises a final concentration of about 1% to about 20% (w/v) of the cryoprotectant or crystalline bulking agent. In a suitable embodiment, the mixture comprises a final concentration of about 3% to about 17%, about 5% to about 15% or about 8% to about 12% (w/v) cryoprotectant or crystalline bulking agent. For example about 8%, about 9%, about 10%, about 11%, or about 12% (w/v) cryoprotectant or crystalline bulking agent.

Suitable for use in the present invention are cryoprotectants and bulking agents including, but not limited to the following sugars: sucrose, lactose, trehalose, maltose or glucose. In a suitable embodiment, the mixture comprises a final concentration of about 1% to about 20% (w/v) sugar. In a suitable embodiment, the mixture comprises about 3% to about 17%, about 5% to about 15% or about 8% to about 12% (w/v) sugar. For example about 8%, about 9%, about 10%, about 11%, or about 12% (w/v) sugar.

In other suitable embodiments the solution contains about 1% to about 20% (w/v) sucrose. For example, the solution contains about 3% to about 17%, about 5% to about 15%, or about 8% to about 12% (w/v) sucrose. For example about 8%, about 9%, about 10%, about 11%, or about 12% (w/v) sucrose. In yet another suitable embodiment, the solution contains about 10% (w/v) sucrose.

The present invention also relates to cell delivery particles, pharmaceutical component-particle dispersions, cell delivery particle compositions or pharmaceutical compositions reconstituted from lyophilized cell delivery particles, pharmaceutical component-particle dispersions, cell delivery particle compositions or pharmaceutical compositions as described above. The lyophilized cell delivery particles, pharmaceutical component-particle dispersions, cell delivery particle compositions or pharmaceutical compositions may be reconstituted with any aqueous solution, such as those described supra. The reconstituted cell delivery particles, pharmaceutical component-particle dispersions, cell delivery particle compositions or pharmaceutical compositions will be a substantially uniform suspension such that a majority of the particles would fall within a Gaussian distribution when the reconstituted solution is examined by serial dilution.

The methods of the present invention are suitable for the manufacture of sterile cell delivery particles, pharmaceutical component-particle dispersions, cell delivery particle compositions or pharmaceutical compositions. All of the components of the cell delivery particles may be sterilized prior to homogenization, the apparatus used for homogenization may be sterilized and the process of homogenization then is performed under sterile conditions. Alternatively, the cell delivery particles produced by the methods of the present invention may be sterilized after particle formation.

Methods of sterilization for use with the present invention include, but are not limited to, filter sterilization or UV irradiation. UV irradiation is a suitable method of sterilization when sterile polynuleotides are added to the cell delivery particles after sterilization. Filter sterilization of the cell delivery particles provides a cost-effective and time-efficient method of sterilization. The filtration step eliminates the need to pre-sterilize the components prior to mixing and performing the homogenization step under sterile conditions. By passing the mixture through a sterile filter with a defined pore size smaller than bacterial pathogens, the solution is sterilized. A wide variety of filter materials which are acceptable for use in sterile filtration devices are known in the art and may be employed. Such materials include, but are not limited to, polyethersulphone, nylon, cellulose acetate, polytetrafluoroethylene, polycarbonate and polyvinylidene. Such materials may be fabricated to provide a filter which has a defined pore size.

The pore size of the filters utilized in the cold filtration step in the present invention are from about 0.01 microns to about 0.3 microns and alternatively from about 0.05 microns to about 0.25 microns. An exemplary pore size of a filter for the filtration step is about 0.05 microns, about 0.1 microns, about 0.15 microns, about 0.2 microns, about 0.25 microns, about 0.3 microns, or about 0.35 microns.

Additional embodiments of the present invention are drawn to cell delivery particles, pharmaceutical component-particle dispersions, cell delivery particle compositions or pharmaceutical compositions comprising an auxiliary agent. As used herein, an “auxiliary agent” is a substance included in a cell delivery particle, pharmaceutical component-particle dispersion, cell delivery particle composition or pharmaceutical composition for its ability to enhance, relative to a cell delivery particle, pharmaceutical component-particle dispersion, cell delivery particle composition or pharmaceutical composition which is identical except for the inclusion of the auxiliary agent, the activity, e.g. cell entry, gene expression, immunogenicity, therapeutic effect and the like, of a cell delivery particle, pharmaceutical component-particle dispersion, cell delivery particle composition or pharmaceutical composition used according to the methods described herein. Auxiliary agents may, for example, enhance entry of a polynucleotide into cells, or enhance an immune response to an immunogen encoded by a polynucleotide delivered to cells. Auxiliary agents of the present invention include nonionic, anionic, cationic, or zwitterionic surfactants or detergents, with nonionic surfactants or detergents being preferred, chelators, DNase inhibitors, poloxamers, agents that aggregate or condense nucleic acids, emulsifying or solubilizing agents, wetting agents, gel-forming agents, and buffers. Auxiliary agents may be combined into cell delivery particles either before or during homogenization, may be mixed with pharmaceutical component-particle dispersions or may be added to a pharmaceutical composition or cell delivery particle composition after formation of cell delivery particles or pharmaceutical component-particle dispersions disclosed herein.

Auxiliary agents for use in compositions of the present invention include, but are not limited to non-ionic detergents and surfactants IGEPAL CA 630®, NONIDET NP40, Nonidet® P40, Tween-20™, Tween-80™, Pluronic® F68 (ave. MW: 8400; approx. MW of hydrophobe, 1800; approx. wt. % of hydrophile, 80%), Pluronic F77® (ave. MW: 6600; approx. MW of hydrophobe, 2100; approx. wt. % of hydrophile, 70%), Pluronic P65® (ave. MW: 3400; approx. MW of hydrophobe, 1800; approx. wt. % of hydrophile, 50%), Triton X-100™, and Triton X-114™; the anionic detergent sodium dodecyl sulfate (SDS); the sugar stachyose; the condensing agent DMSO; and the chelator/DNAse inhibitor EDTA, CRL-1005 (12 kDa, 5% POE), and BAK (Benzalkonium chloride 50% solution, available from Ruger Chemical Co. Inc.). In certain specific embodiments, the auxiliary agent is DMSO, Nonidet P40, Pluronic F68® (ave. MW: 8400; approx. MW of hydrophobe, 1800; approx. wt. % of hydrophile, 80%), Pluronic F77® (ave. MW: 6600; approx. MW of hydrophobe, 2100; approx. wt. % of hydrophile, 70%), Pluronic P65® (ave. MW: 3400; approx. MW of hydrophobe, 1800; approx. wt. % of hydrophile, 50%), Pluronic L64® (ave. MW: 2900; approx. MW of hydrophobe, 1800; approx. wt. % of hydrophile, 40%), and Pluronic F108® (ave. MW: 14600; approx. MW of hydrophobe, 3000; approx. wt. % of hydrophile, 80%). See, e.g., U.S. Patent Application Publication No. 2002/0019358, published Feb. 14, 2002, which is incorporated herein by reference in its entirety.

Cell delivery compositions, pharmaceutical component-particle dispersions or pharmaceutical compositions produced by the methods of the present invention which contain polynucleotides may also optionally include a non-ionic surfactant, such as polysorbate-80, which may be useful to control particle aggregation in the presence of the polynucleotide. Additional non-ionic surfactants are known in the art and may be used to practice the invention. These additional non-ionic surfactants include, but are not limited to, other polysorbates, -Alkylphenyl polyoxyethylene ether, n-alkyl polyoxyethylene ethers (e.g., Tritons™), sorbitan esters (e.g., Spans™), polyglycol ether surfactants (Tergitol™), polyoxyethylenesorbitan (e.g., Tweens™), poly-oxyethylated glycol monoethers (e.g., Brij™, polyoxylethylene 9 lauryl ether, polyoxyethylene 10 ether, polyoxylethylene 10 tridecyl ether), lubrol, perfluoroalkyl polyoxylated amides, N,N-bis [3D-gluconamidopropyl]cholamide, decanoyl-N-methylglucamide, -decyl β-D-glucopyranozide, n-decyl β-D-glucopyranozide, n-decyl β-D-maltopyanozide, n-dodecyl β-D-glucopyranozide, n-undecyl β-D-glucopyranozide, n-heptyl β-D-glucopyranozide, n-heptyl β-D-thioglucopyranozide, n-hexyl β-D-glucopyranozide, n-nonanoyl β-glucopyranozide 1-monooleyl-rac-glycerol, nonanoyl-N-methylglucamide, -dodecyl β-D-maltoside, N,N bis [3-gluconamidepropyl]deoxycholamide, diethylene glycol monopentyl ether, digitonin, hepanoyl-N-methylglucamide, octanoyl-N-methylglucamide, n-octyl βD-glucopyranozide, n-octyl β-D-glucopyranozide, n-octyl β-D-thiogalactopyranozide, n-octyl β-D-thioglucopyranozide.

Certain pharmaceutical component-particle dispersions or pharmaceutical compositions of the present invention may further include one or more adjuvants which are administered before, after, or concurrently with the pharmaceutical component-particle dispersions or pharmaceutical compositions of the invention. The term “adjuvant” refers to any material having the ability to (1) alter or increase an immune response to a particular antigen or (2) increase or aid an effect of a pharmacological agent. It should be noted, with respect to the present pharmaceutical component-particle dispersions or pharmaceutical compositions, that an “adjuvant,” may be a component of a cell delivery particle produced as described supra, e.g. an amphiphilic composition or block copolymer. Suitable adjuvants include, but are not limited to, cytokines and growth factors; bacterial components (e.g., endotoxins, in particular superantigens, exotoxins and cell wall components); aluminum-based salts; calcium-based salts; silica; polynucleotides; toxoids; serum proteins, viruses and virally-derived materials, poisons, venoms, imidazoquiniline compounds, poloxamers, and cationic lipids.

A great variety of materials have been shown to have adjuvant activity through a variety of mechanisms. Any compound which may increase the expression, antigenicity or immunogenicity of the pharmaceutical component is a potential adjuvant. Potential adjuvants which may used in the present invention include, but are not limited to: inert carriers, such as alum, bentonite, latex, and acrylic particles; pluronic block polymers, such as TiterMax® (block copolymer CRL-8941, squalene (a metabolizable oil) and a microparticulate silica stabilizer), depot formers, such as Freunds adjuvant, surface active materials, such as saponin, lysolecithin, retinal, Quil A, liposomes, and pluronic polymer formulations; macrophage stimulators, such as bacterial lipopolysaccharide; alternate pathway complement activators, such as insulin, zymosan, endotoxin, and levamisole; and non-ionic surfactants, such as poloxamers, poly(oxyethylene)-poly(oxypropylene) tri-block copolymers.
Methods of treating or preventing disease, generating an immune response or in vitro delivery of a pharmaceutical component.

The invention further relates to methods for generating a detectible immune response in a vertebrate by administration one or more cell delivery particles, pharmaceutical component-particle dispersions, cell delivery particle compositions or pharmaceutical compositions produced by the methods of the present invention to a vertebrate. In another embodiment the invention relates to methods for treating or preventing a disease or condition in a vertebrate by administering one or more cell delivery particles, pharmaceutical component-particle dispersions, cell delivery particle compositions or pharmaceutical compositions of the present invention to a vertebrate. Additionally, the invention relates to methods for delivering a component (e.g. a pharmaceutically active drug, an antigenic molecule, or a polynucleotide), to a cell in vitro, comprising contacting one or more cell delivery particles, pharmaceutical component-particle dispersions, cell delivery particle compositions or pharmaceutical compositions of the invention to cells.

Determining an effective amount of the cell delivery particles, pharmaceutical component-particle dispersions, cell delivery particle compositions or pharmaceutical compositions of the invention depends upon a number of factors including, for example, the chemical structure and biological activity of the pharmaceutical component, if any, to be delivered, the age and weight of the subject, and the route of administration or the type of cells in culture. The precise amount, number of doses, and timing of doses can be readily determined by those skilled in the art.

Any route of delivery of the cell delivery particles, pharmaceutical component-particle dispersions, cell delivery particle compositions and pharmaceutical compositions is contemplated by the present invention. Routes of administration include but are not limited to intramuscular administration, intratracheal administration, intranasal administration, transdermal administration, interdermal administration, subcutaneous administration, intraocular administration, vaginal administration, rectal administration, intraperitoneal administration, intraintestinal administration, oral administration (e.g. inhalation), intervenous administration or topical administration.

The cell delivery particles, pharmaceutical component-particle dispersions, cell delivery particle compositions and pharmaceutical compositions of the invention may be delivered to the interstitial space of tissues of the animal body, including those of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels.

In certain embodiments, cell delivery particles, pharmaceutical component-particle dispersions, cell delivery particle compositions or pharmaceutical compositions of the invention comprise a polynucleotide, e.g. a polynucleotide encoding a therapeutic immunogenic polypeptide. Such compositions may be administered to a body cavity such as lungs, the mouth, the nasal cavity, the stomach, the peritoneal cavity, the intestine, a heart chamber, veins, arteries, capillaries, lymphatic cavities, the uterine cavity, the vaginal cavity, the rectal cavity, joint cavities, ventricles in brain, spinal canal in spinal cord, and the ocular cavities.

A tissue can also serve as the site of administration or delivery of cell delivery particles, pharmaceutical component-particle dispersions, cell delivery particle compositions or pharmaceutical compositions of the invention. Non-limiting examples of such tissues include: muscle, skin, brain tissue, lung tissue, liver tissue, spleen tissue, bone marrow tissue, thymus tissue, heart tissue, lymph tissue, blood tissue, bone tissue, connective tissue, mucosal tissue, pancreas tissue, kidney tissue, gall bladder tissue, intestinal tissue, testicular tissue, ovarian tissue, uterine tissue, vaginal tissue, rectal tissue, nervous system tissue, eye tissue, glandular tissue, and tongue tissue.

Any mode of administration can be used so long as the administration results in desired immune response. Administration means of the present invention include, but not limited to, needle injection, catheter infusion, biolistic injectors, particle accelerators (i.e., “gene guns” or pneumatic “needleless” injectors—for example, Med-E-Jet (Vahlsing, H., et al., J. Immunol. Methods 171, 11-22 (1994)), Pigjet (Schrijver, R., et al., Vaccine 15, 1908-1916 (1997)), Biojector (Davis, H., et al., Vaccine 12, 1503-1509 (1994); Gramzinski, R., et al., Mol. Med. 4, 109-118 (1998)), AdvantaJet, Medijector, gelfoam sponge depots, other commercially available depot materials (e.g., hydrojels), osmotic pumps (e.g., Alza minipumps), oral or suppositorial solid (tablet or pill) pharmaceutical formulations, topical skin creams, and decanting, use of coated suture (Qin et al., Life Sciences 65, 2193-2203 (1999)) or topical applications during surgery. Other modes of administration include intramuscular needle-based injection and intranasal application as an aqueous solution.

In certain embodiments, the cell delivery particles, pharmaceutical component-particle dispersions, cell delivery particle compositions or pharmaceutical compositions described above can be formulated according to known methods, whereby a pharmaceutical component-particle dispersion is combined with a pharmaceutically acceptable carrier vehicle to form a pharmaceutical composition. Suitable vehicles and their preparation are described, for example, in Remington's Pharmaceutical Sciences, 16th Edition, A. Osol, ed., Mack Publishing Co., Easton, Pa. (1980), and Remington's Pharmaceutical Sciences, 19^{th} Edition, A. R. Gennaro, ed., Mack Publishing Co., Easton, Pa. (1995) and supra. The pharmaceutical composition can be formulated as an emulsion, gel, solution, suspension, lyophilized form, or any other form known in the art. In addition, the pharmaceutical composition can also contain pharmaceutically acceptable additives including, for example, diluents, binders, stabilizers, and preservatives.

For aqueous pharmaceutical compositions used in vivo, use of sterile pyrogen-free water is preferred. Such formulations will contain an effective amount of the pharmaceutical component-particle dispersion together with a suitable amount of a pharmaceutically acceptable carrier vehicle in order to prepare pharmaceutically acceptable compositions suitable for administration to a vertebrate.

The pharmaceutical component-particle dispersions or pharmaceutical compositions of the present invention may include a therapeutic polypeptide or polynucleotide encoding a therapeutic polypeptide. As used herein, a “therapeutic polypeptide” is a polypeptide which when delivered to a vertebrate, treats, i.e., cures, ameliorates, or lessens the symptoms of, a given disease in that vertebrate, or alternatively, prolongs the life of the vertebrate by slowing the progress of a terminal disease.

Additionally, the pharmaceutical component-particle dispersions or pharmaceutical compositions of the present invention may include an immunomodulatory polypeptide or polynucleotide encoding such a polypeptide. As used herein, an “immunomodulatory polypeptide” is a polypeptide which, when delivered to a vertebrate, can alter, enhance, suppress, or regulate an immune response in a vertebrate. Immunomodulatory polypeptides are a subset of therapeutic polypeptides. Therapeutic and immunomodulatory polypeptides of the present invention include, but are not limited to, cytokines, chemokines, lymphokines, ligands, receptors, hormones, apoptosis-inducing polypeptides, enzymes, antibodies, and growth factors. Examples include, but are not limited to granulocyte macrophage colony stimulating factor (GM-CSF), granulocyte colony stimulating factor (G-CSF), macrophage colony stimulating factor (M-CSF), colony stimulating factor (CSF), interleukin 2 (IL-2), interleukin-3 (IL-3), interleukin 4 (IL-4), interleukin 5 (IL-5), interleukin 6 (IL-6), interleukin 7 (IL-7), interleukin 8 (IL-8), interleukin 10 (IL-10), interleukin 12 (IL-12), interleukin 15 (IL-15), interleukin 18 (IL-18), interferon alpha (IFNα), interferon beta (IFNβ), interferon gamma (IFNγ), interferon omega (IFNα), interferon tau (IFNτ), interferon gamma inducing factor I (IGIF), transforming growth factor beta (TGF-β), RANTES (regulated upon activation, normal T-cell expressed and presumably secreted), macrophage inflammatory proteins (e.g., MIP-1 alpha and MIP-1 beta), Leishmania elongation initiating factor (LEIF), platelet derived growth factor (PDGF), tumor necrosis factor (TNF), growth factors, e.g., epidermal growth factor (EGF), vascular endothelial growth factor (VEGF), fibroblast growth factor, (FGF), nerve growth factor (NGF), brain derived neurotrophic factor (BDNF), neurotrophin-2 (NT-2), neurotrophin-3 (NT-3), neurotrophin-4 (NT-4), neurotrophin-5 (NT-5), glial cell line-derived neurotrophic factor (GDNF), ciliary neurotrophic factor (CNTF), erythropoietin (EPO), and insulin.

Therapeutic polypeptides, and polynucleotides encoding such polypeptides, in combination with the pharmaceutical component-particle dispersions or pharmaceutical compositions of the present invention may be used to treat diseases such as Parkinson's disease, cancer, and heart disease. In addition, compositions of the present invention comprising therapeutic polypeptides, or polynucleotides encoding therapeutic polypeptides, may be used to treat acute and chronic inflammatory disorders, to promote wound healing, to prevent rejection after transplantation of cells, tissues, or organs; and autoimmune disorders such as multiple sclerosis; Sjogren's syndrome; sarcoidosis; insulin dependent diabetes mellitus; autoimmune thyroiditis; arthritis (e.g.), osteoarthritis, rheumatoid arthritis, reactive arthritis, and psoriatic arthritis; ankylosing spondylitis; scleroderma. Therapeutic polypeptides to promote wound healing such as growth factors, include, but are not limited to, FGF and EGF.

In conjunction with the pharmaceutical component-particle dispersions or pharmaceutical compositions of the present invention, therapeutic polypeptides and polynucleotides which encode said polypeptides, such as neurotrophic factors (NTFs), may be used to promote the survival, maintenance, differentiation, repair, regeneration, and growth of cells in the brain, spinal cord, and peripheral nerves. Suitable NTFs include, but are not limited to, NGF, BDNF, the Neurotrophins or NTs such as NT-2, NT-3, NT-4, NT-5, GDNF, CNTF, as well as others. The administration of purified recombinant NTFs represents a clinical strategy for treatment of such acute and chronic nervous system disorders. Such disorders include, but are not limited to mechanical or chemical brain or spinal cord injury, Parkinson's Disease, Alzheimer's Disease and other dementias, Amyotrophic Lateral Sclerosis and Multiple Sclerosis.

Therapeutic polypeptides and polynucleotides encoding the polypeptides may be used in conjunction with the pharmaceutical component-particle dispersions or pharmaceutical compositions of the present invention to promote cell suicide (termed “apoptosis”). Suitable apoptotic polypeptides include the BAX protein. Alternatively, the compositions of the present invention may be used to prevent apoptosis. Suitable apoptosis antagonists include the BAX antagonist Bcl-2. A disease which may be treated with apoptosis-inhibiting polypeptides is Muscular Dystrophy (MD), where patients have a defective protein called Dystrophin. Dystrophin is required for proper muscle function. The non-defective, normal Dystrophin may act as an antigen if delivered via plasmid DNA to patients with MD. In this case, muscle cells transduced with DNA encoding normal Dystrophin would be recognized by the immune system and killed by Dystrophin-specific T cell based responses. Such T cell based killing is known to kill cells by inducing apoptosis. If the normal, and potentially immunogenic, Dystrophin could be delivered into muscle cells along with Bcl-2 or other apoptosis-preventing protein, one would expect that CTL would be unable to kill the muscle cells. This reasoning applies to many genetic diseases where treatment involves delivery of a “normal”, and therefore potentially immunogenic, copy of a protein.

Polynucleotides encoding functional self polypeptides as well as the polypeptides may be used in the pharmaceutical component-particle dispersions or pharmaceutical compositions of the present invention. As used herein, a “functional self polypeptide” is a polypeptide which is required for normal functioning of a vertebrate, but because of, e.g., genetic disease, cancer, environmental damage, or other cause, is missing, defective, or non-functional in a given individual. A composition of the present invention is used to restore the individual to a normal state by supplying the necessary polypeptide. Examples of functional self polypeptides include insulin, dystrophin, cystic fibrosis transmembrane conductance regulator, granulocyte macrophage colony stimulating factor, granulocyte colony stimulating factor, macrophage colony stimulating factor colony stimulating factor, interleukin 2, interleukin-3, interleukin 4, interleukin 5, interleukin 6, interleukin 7, interleukin 8, interleukin 10, interleukin 12, interleukin 15, interleukin 18, interferon alpha, interferon beta, interferon gamma, interferon omega, interferon tau, interferon gamma inducing factor I, transforming growth factor beta, RANTES, Flt-3 ligand, macrophage inflammatory proteins, platelet derived growth factor, tumor necrosis factor, epidermal growth factor, vascular epithelial growth factor, fibroblast growth factor, insulin-like growth factors I and II, insulin-like growth factor binding proteins, nerve growth factor, brain derived neurotrophic factor, neurotrophin-2, neurotrophin-3, neurotrophin-4, neurotrophin-5, glial cell line-derived neurotrophic factor, ciliary neurotrophic factor, and erythropoietin. Examples of diseases or disorders that may be treated with functional self polypeptides include, but are not limited to: diabetes, muscular dystrophy, multiple sclerosis, Parkinson's disease, Alzheimer's disease, arthritis, sickle cell anemia, and hemophilia.

Examples of antigenic and immunogenic polypeptides include, but are not limited to, polypeptides from infectious agents such as bacteria, viruses, parasites, or fungi, allergens such as those from pet dander, plants, dust, and other environmental sources, as well as certain self polypeptides, for example, tumor-associated antigens.

Antigenic and immunogenic molecules in the pharmaceutical component-particle dispersions or pharmaceutical compositions of the present invention can be used to prevent or treat, i.e., cure, ameliorate, lessen the severity of, or prevent or reduce contagion of viral, bacterial, fungal, and parasitic infectious diseases, as well as to treat allergies.

In addition, antigenic and immunogenic molecules can be used in the pharmaceutical component-particle dispersions or pharmaceutical compositions of the present invention to prevent or treat, i.e., cure, ameliorate, or lessen the severity of cancer including, but not limited to, cancers of oral cavity and pharynx (i.e., tongue, mouth, pharynx), digestive system (i.e., esophagus, stomach, small intestine, colon, rectum, anus, anal canal, anorectum, liver, gallbladder, pancreas), respiratory system (i.e., larynx, lung), bones, joints, soft tissues (including heart), skin, melanoma, breast, reproductive organs (i.e., cervix, endometirum, ovary, vulva, vagina, prostate, testis, penis), urinary system (i.e., urinary bladder, kidney, ureter, and other urinary organs), eye, brain, endocrine system (i.e., thyroid and other endocrine), lymphoma (i.e., hodgkin's disease, non-hodgkin's lymphoma), multiple myeloma, leukemia (i.e., acute lymphocytic leukemia, chronic lymphocytic leukemia, acute myeloid leukemia, chronic myeloid leukemia).

Examples of viral antigenic and immunogenic polypeptides include, but are not limited to, adenovirus polypeptides, alphavirus polypeptides, calicivirus polypeptides, e.g., a calicivirus capsid antigen, coronavirus polypeptides, distemper virus polypeptides, Ebola virus polypeptides, enterovirus polypeptides, flavivirus polypeptides, hepatitis virus (AE) polypeptides, e.g., a hepatitis B core or surface antigen, herpesvirus polypeptides, e.g., a herpes simplex virus or varicella zoster virus glycoprotein, immunodeficiency virus polypeptides, e.g., the human immunodeficiency virus envelope or protease, infectious peritonitis virus polypeptides, influenza virus polypeptides, e.g., an influenza A hemagglutinin, neuraminidase, or nucleoprotein, leukemia virus polypeptides, Marburg virus polypeptides, orthomyxovirus polypeptides, papilloma virus polypeptides, parainfluenza virus polypeptides, e.g., the hemagglutinin/neuraminidase, paramyxovirus polypeptides, parvovirus polypeptides, pestivirus polypeptides, picoma virus polypeptides, e.g., a poliovirus capsid polypeptide, pox virus polypeptides, e.g., a vaccinia virus polypeptide, rabies virus polypeptides, e.g., a rabies virus glycoprotein G, reovirus polypeptides, retrovirus polypeptides, and rotavirus polypeptides.

Examples of bacterial antigenic and immunogenic polypeptides include, but are not limited to, Actinomyces polypeptides, Bacillus polypeptides, Bacteroides polypeptides, Bordetella polypeptides, Bartonella polypeptides, Borrelia polypeptides, e.g., B. burgdorferi OspA, Brucella polypeptides, Campylobacter polypeptides, Capnocytophaga polypeptides, Chlamydia polypeptides, Clostridium polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides, e.g., H. influenzae type b outer membrane protein, Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides, Proteus polypeptides, Pseudomonas polypeptides, Rickettsia polypeptides, Rochalimaea polypeptides, Salmonella polypeptides, Shigella polypeptides, Staphylococcus polypeptides, Streptococcus polypeptides, e.g., S. pyogenes M proteins, Treponema polypeptides, and Yersinia polypeptides, e.g., Y. pestis F1 and V antigens.

Examples of fungal immunogenic and antigenic polypeptides include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite immunogenic and antigenic polypeptides include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides, e.g., P. falciparum circumsporozoite (PfCSP), sporozoite surface protein 2 (PfSSP2), carboxyl terminus of liver state antigen 1 (PfLSAl c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of helminth parasite immunogenic and antigenic polypeptides include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides.

Examples of ectoparasite immunogenic and antigenic polypeptides include, but are not limited to, polypeptides (including protective antigens as well as allergens) from fleas; ticks, including hard ticks and soft ticks; flies, such as midges, mosquitos, sand flies, black flies, horse flies, horn flies, deer flies, tsetse flies, stable flies, myiasis-causing flies and biting gnats; ants; spiders, lice; mites; and true bugs, such as bed bugs and kissing bugs.

Examples of tumor-associated antigenic and immunogenic polypeptides include, but are not limited to, tumor-specific immunoglobulin variable regions (e.g., B cell lymphoma idiotypes), GM2, Tn, sTn, Thompson-Friedenreich antigen (TF), Globo H, Le(y), MUC1, MUC2, MUC3, MUC4, MUC5AC, MUC5B, MUC7, carcinoembryonic antigens, beta chain of human chorionic gonadotropin (hCG beta), HER2/neu, PSMA, EGFRvIII, KSA, PSA, PSCA, GP100, MAGE 1, MAGE 2, TRP 1, TRP 2, tyrosinase, MART-1, PAP, CEA, BAGE, MAGE, RAGE, and related proteins.

Also included as polypeptides and polynucleotides for use in the pharmaceutical component-particle dispersions or pharmaceutical compositions of the present invention are fragments, derivatives, analogs, or variants of the foregoing polypeptides and polynucleotides, and any combination of the foregoing polypeptides. Additional polypeptides may be found, for example in “Foundations in Microbiology,” Talaro, et al., eds., McGraw-Hill Companies (October, 1998), Fields, et al., “Virology,” 3d ed., Lippincott-Raven (1996), “Biochemistry and Molecular Biology of Parasites,” Marr, et al., eds., Academic Press (1995), and Deacon, J., “Modem Mycology,” Blackwell Science Inc (1997), which are incorporated herein by reference.

Other polynucleotides for use in the pharmaceutical component-particle dispersions or pharmaceutical compositions of the present invention include functional RNAs (e.g. tRNA or rRNA) which may replace a defective or deficient endogenous functional RNAs. Additional RNAs for use in the present invention include RNA's described supra.

The methods of the invention may be applied by direct administration of the cell delivery particles, pharmaceutical component-particle dispersions, cell delivery particle compositions or pharmaceutical compositions into the vertebrate in vivo, or by in vitro transfection of cells which are then administered to the vertebrate.

In additional embodiments, the invention relates to a method for delivering a pharmaceutical component or other molecules to a cell in vitro. Such pharmaceutical components include but are not limited to polynucleotides, antigenic molecules and pharmaceutically active drugs such as those described supra. The cell delivery particles, pharmaceutical component-particle dispersions, cell delivery particle compositions or pharmaceutical compositions of the invention may be incubated with any type of cell in tissue culture according to methods known in the art.

The length of incubation may vary depending upon transfection efficiency of the cells, amount and components in the composition and volume used. One of skill in the art would be able to adjust the time depending upon the composition, cells and results desired.

Kits

The invention further provides for kits comprising the cell delivery particles, pharmaceutical component-particle dispersions, cell delivery particle compositions or pharmaceutical compositions produced by the methods of the invention. In certain embodiments, the kits comprise cell delivery particles, pharmaceutical component-particle dispersions, cell delivery particle compositions or pharmaceutical compositions produced by the methods of the invention for use in delivering a pharmaceutical component to a vertebrate. The cell delivery particles, pharmaceutical component-particle dispersions, cell delivery particle compositions or pharmaceutical compositions may be prepared in unit dosage form in ampules, or in multidose containers. The cell delivery particles, pharmaceutical component-particle dispersions, cell delivery particle compositions or pharmaceutical compositions may be present in such forms as suspensions, solutions, or preferably aqueous vehicles. Alternatively, the cell delivery particles, pharmaceutical component-particle dispersions, cell delivery particle compositions or pharmaceutical compositions may be in lyophilized form for reconstitution, at the time of delivery, with a pharmaceutically acceptable carrier vehicle, e.g. sterile pyrogen-free water. Both liquid as well as lyophilized forms that are to be reconstituted may comprise agents, preferably buffers, in amounts necessary to suitably adjust the pH of the injected solution as described herein. For any parenteral use, particularly if the cell delivery particles, pharmaceutical component-particle dispersions, cell delivery particle compositions or pharmaceutical compositions is to be administered intravenously, the total concentration of solutes should be controlled to make the preparation isotonic, hypotonic, or weakly hypertonic. Nonionic materials, such as sugars, may be used to adjust tonicity, for example sucrose. Any of these forms may further comprise suitable formulatory agents, such as starch or sugar, glycerol or saline. The pharmaceutical component-particle dispersions or pharmaceutical compositions per unit dosage, whether liquid or solid, may contain from 0.1% to 99% of a pharmaceutical component.

Each kit includes a container holding about 1 ng to about 30 mg of a cell delivery particle, pharmaceutical component-particle dispersion, cell delivery particle composition or pharmaceutical composition. Preferably, the kit includes from about 100 ng to about 10 mg of a polynucleotide or other pharmaceutical component component. In alternative embodiments, each kit includes, in the same or in a different container, an adjuvant composition. Any components of the pharmaceutical kits can be provided in a single container or in multiple containers.

Any suitable container or containers may be used with pharmaceutical kits. Examples of containers include, but are not limited to, glass containers, plastic containers, or strips of plastic or paper.

Each of the pharmaceutical kits may further comprise an administration means. Means for administration include, but are not limited to syringes and needles, catheters, biolistic injectors, particle accelerators, i.e., “gene guns,” pneumatic “needleless” injectors, gelfoam sponge depots, other commercially available depot materials, e.g., hydrojels, osmotic pumps, and decanting or topical applications during surgery. Each of the pharmaceutical kits may further comprise sutures, e.g., coated with the immunogenic composition (Qin et al., Life Sciences (1999) 65:2193-2203).

The kit can further comprise an instruction sheet for administration of the cell delivery particles, pharmaceutical component-particle dispersions, cell delivery particle compositions or pharmaceutical compositions to a vertebrate. The cell delivery particles, pharmaceutical component-particle dispersions, cell delivery particle compositions or pharmaceutical compositions are preferably provided as a liquid solution or in lyophilized form. Various components of the cell delivery particles, pharmaceutical component-particle dispersions, cell delivery particle compositions or pharmaceutical compositions maybe lyophilized together or separately. Such a kit may further comprise a container with an exact amount of sterile pyrogen-free water or other aqueous solution, for precise reconstitution of the lyophilized components of the cell delivery particles, pharmaceutical component-particle dispersions, cell delivery particle compositions or pharmaceutical compositions.

The container in which the pharmaceutical composition is packaged prior to use can comprise a hermetically sealed container enclosing an amount of the lyophilized cell delivery particles, pharmaceutical component-particle dispersions, cell delivery particle compositions or pharmaceutical compositions or a solution containing the cell delivery particles, pharmaceutical component-particle dispersions, cell delivery particle compositions or pharmaceutical compositions suitable for a pharmaceutically effective dose thereof, or multiples of an effective dose. The cell delivery particles, pharmaceutical component-particle dispersions, cell delivery particle compositions or pharmaceutical compositions is packaged in a sterile container, and the hermetically sealed container is designed to preserve sterility of the pharmaceutical formulation until use. Optionally, the container can be associated with administration means and/or instruction for use.

These example and equivalents thereof will become more apparent to those skilled in the art in light of the present disclosure and the accompanying claims. It should be understood, however, that the examples are designed for the purpose of illustration only and not limiting of the scope of the invention in any way. All patents and publications cited herein are fully incorporated by reference herein in their entirety.

### EXAMPLES

### General Methods

Benzalkonium Chloride

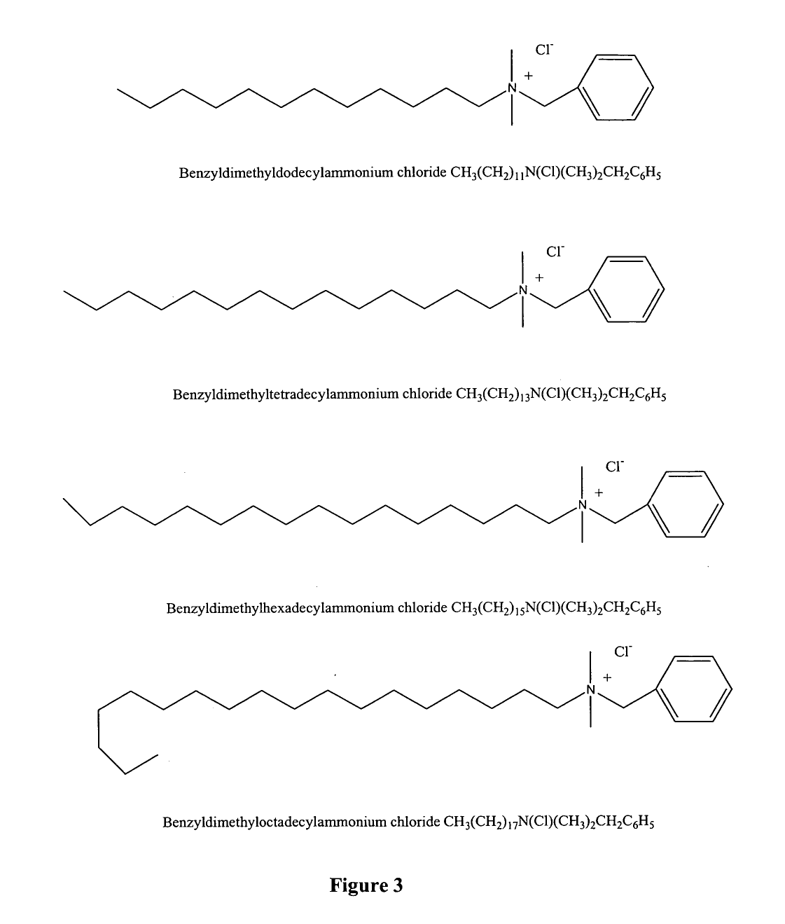

Benzalkonium chloride (BAK) is a commercially available mixture of four homologs with the hydrocarbon chain lengths of 12 carbons (N-benzyl-N,N-dimethyl-N-dodecyl-ammonium chloride), 14 carbons (N-benzyl-N,N-dimethyl-N-teradecyl-ammonium chloride), 16 carbons (N-benzyl-N,N-dimethyl-N-hexadecyl-ammonium chloride) and 18 (N-benzyl-N,N-dimethyl-N-octadecyl-ammonium chloride) carbons. See FIG. 3. Two commonly used, commercially available BAK solutions are BTC 50 NF and BTC 65 NF. The relative amounts of each homolog found in the BTC 50 NF and BTC 65 NF formulations are listed in Table 1. Formulations of DNA, CRL-1005 and BAK have previously been made using BTC 65 NF as the BAK component with the thermal cycling method described in Published International Patent Application No. WO 02/00844 A2. Formulations of DNA, CRL-1005 and BAK have previously been made using BTC 50 NF as the BAK component using the thermal cycling method described in U.S. Published Patent Applications 2004/0162256 A1 and 2004/0209241 A1, which are both herein incorporated by reference in their entireties. Using this method, precipitation of the DNA in the solution occurs at BAK concentrations of 0.6 mM and above. Studies using the individual benzalkonium chloride analogs found that the C₁₂ homolog does not interact with poloxamer, the C₁₄ and C₁₆ homologs interact with poloxamer to produce submicron particles with a positive surface charge, and the C₁₈ homolog does interact with poloxamer to produce large micron sized particles with a positive surface charge. See Example 1. When DNA, poloxamer and BAK C₁₄ or C₁₆ were formulated using the thermal cycling method, described in U.S. Published Patent Applications 2004/0162256 A1 and 2004/0209241 A1, comparable formulations to those produced using BTC 50 NF were produced. See Example 1. However at concentrations above 0.3 mM, the BAK C₁₆ homolog caused precipitation of the DNA and the formation of visible particulates in the formulation. See Example 1. The previously described thermal cycling method requires the amphiphilic component of the composition to be soluble below the cloud point of the poloxamer and therefore it is not possible to produce stable cell delivery particles using high concentrations of BAK or the BAK C₁₈ homolog or cationic lipids such as DMRIE and VC 1052. An alternative approach to formulate compositions comprising these components is to use homogenization at temperatures above the cloud point of the poloxamer.

TABLE 1
Percentage of each BAK homolog found in
BAK solutions BTC 50 NF and BTC 65 NF.

| BAK homolog | BTC 50 NF | BTC 65 NF |
|---|---|---|
| % C₁₂ | 50 | 67 |
| % C₁₄ | 30 | 25 |
| % C₁₆ | 17 | 7 |
| % C₁₈ | 3 | 1 |

Homogenization

Hydrophobic high molecular weight poloxamers such as CRL-1005 and CRL-8300 have inverse solubility characteristics in aqueous media. Below their cloud points (7-12° C.), these block copolymers are water-soluble and form clear solutions that can be sterile filtered. The solution process involves the formation of hydrogen bonds between oxygen atoms and hydroxyl groups in the block copolymer and water molecules. When a solution of block copolymer is warmed and passes through its cloud point, the increased thermal motion is sufficient to break the hydrogen bonds between the water and the block copolymer. As the block copolymer comes out of solution, the block copolymer molecules self-assemble into particulates. This process is reversible. Solutions of CRL-8300 and CRL-1005 at 7.5 mg/ml in PBS above their cloud points form particles which are greater than 1 micron in diameter, as measured by photon correlation spectroscopy. See Examples 1 and 2. When these solutions are homogenized at 15,000 psi, at 15° C., for fifteen passes through the homogenizer valve, both block copolymers form particles of less than 300 nm in diameter (See Examples 1 and 2) with a negative surface charge, as measured by micro-electrophoresis. When these poloxamers are homogenized in the presence of a cationic lipids such as: DMRIE (See Examples 2-7 and 8) VC1052 (See Example 7) or BAK C₁₈ (See example 12), a combination of lipids such as: DMRIE:DOPE (See Example 11), or BTC 50 NF (See Example 2), submicron particles are produced with a positive surface charge. When DNA is added to these compositions stable sub-micron particles are produced with a negative surface charges (See Examples 3,4,5 to 12).

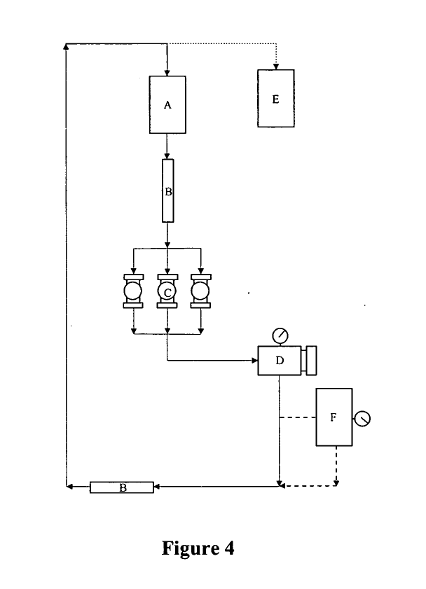

An Avestin Inc., EmulsiFlex-C50 high-pressure homogenizer was used for all experiments. The EmulsiFlex-C50 is described in FIG. 4. The high-pressure homogenizer consists of a high-pressure pump (C) which pushes the product from a reservoir (A) through a heat exchanger (B) into an adjustable homogenizing valve (D). The product is then passed through a second heat exchanger (B) and recycled to the reservoir (A) or collected in a second container (E). The homogenizer can be fitted with an optional filter/extruder (F) down stream from the homogenizer valve (See FIG. 4).

Lyophilization

Lyophilization represents a method by which the cell delivery particle may be stored for extended periods of time and then reconstituted prior to use. However, it is important that the particle size distribution of the formulation should not change during this process. Previously we have shown that using 10% sucrose, 10 mM NaP as the vehicle, the thermal cycling process produces a uniform particle size distribution consistent with that produced previously using PBS as the vehicle. When these formulations were lyophilized and reconstituted in sterile water for injection, the uniform particle size distribution was maintained. When poloxamers are homogenized in the presence of a cationic lipid such as DMRIE or VC1052 and the final vehicle is 8.5% sucrose, these formulations can be lyophilized. See Examples 9 and 10. When these formulations are reconstituted in sterile water for injection, the uniform particle size distribution was maintained provided the formulation was flash frozen in liquid nitrogen prior to lyophilization. See Examples 9 and 10.

Poloxamer Solutions

The required amount of poloxamer was weighed and dispensed into a round bottom flask, the required aqueous media (e.g. sterile water for injection, PBS, 2×PBS or 17% sucrose) was then added and the solution was stirred in an ice bath until the poloxamer was dissolved. The resulting solution was then cold filter sterilized (4° C.) using a steriflip 50 ml disposable vacuum filtration device with a 0.22 μm Millipore express membrane (cat # SCGP00525) and warmed to room temperature ready for use.

Cationic Lipid Solutions

The required amount of lipid was weighed and dispensed into a round bottom flask. The required aqueous media (e.g. sterile water for injection or PBS) was then added and the solution was stirred at 45° C. in a water bath until the lipid was dissolved. The solution was then allowed to cool to room temperature for use.

Particle Size Measurements Using Photon Correlation Spectroscopy

The following examples employ a Malvern 3000 HS Zetasizer to measure particle size. Particles with diameters that range from 1 to 5000 nm, can be measured using the method of photon correlation spectroscopy (PCS). Particles in this size range are in constant random thermal (or Brownian) motion. This motion causes the intensity of light scattered from the particles to form a moving speckle pattern which, with the use of optics and a photomultiplier, can be detected as a change in intensity with time. Large particles move more slowly than small particles, therefore the rate of fluctuation of light scattered from large particles is slower. PCS uses the rate of change of these light fluctuations to determine the size distribution of the particles scattering light. Data is plotted as the auto-correlation function (counts per correlator against delay time). Analysis of this function obtained over time, with a sufficient number of data points, enables the translational diffusion coefficient of the particles undergoing Brownian motion to be calculated. From this coefficient, together with the temperature and viscosity of the suspending liquid, the particle size can be calculated. For aqueous dispersions, the default set-up of the Malvern 3000 HS Zetasizer calculates the viscosity from the temperature. The best single measurement to describe the size of a poloxamer formulation is the mean Z average or the hydrodynamic diameter, which is calculated using cumulants analysis. The polydispersity describes the width of the distribution.

Surface Charge Measurements Using Microelectrophoresis

Zeta potential or surface charge can be readily measured using laser Doppler electrophoresis, called simply microelectrophoresis. This technique measures the movement of colloidal particles when they are placed in an electric field. The measurement can be used to determine the sign of the charge on the particles and also their electrophoretic mobility, which is related to the zeta potential. A pair of mutually coherent laser beams derived from a single source and following similar path lengths are arranged so that the beam paths cross. Scattered light from the crossover region is detected by a detector placed either on the bisector of the crossing angle (Doppler difference), or looking along one of the beams, which in this case must be attenuated. This latter arrangement is referred to as a reference beam or heterodyne measurement, and is used in the Malvern 3000 HS Zetasizer to measure zeta potential. Interference fringes are produced in the crossover region by particles. The spacing of these fringes (s) will give rise to a certain frequency component in the scattered light. For a particle of velocity v, the frequency will be equal to (v)·(s). The autocorrelation function of the scattered light is measured, which for a single velocity has the form of a cosine function whose frequency is (v)·(s). For a spread of velocities, as will arise from a spread of electrophoretic mobilities, or particles undergoing Brownian motion, the cosine wave will be damped. The cosine wave is superimposed on a background from the uncorrelated part of the signal. The signal processing involved requires the Fourier transform of the varying part of the autocorrelation function, the resulting frequency spectrum (translated to electrophoretic mobility) and zeta potential.

ELISPOT Assay

T cell responses against the nucleoprotein expressed by the VR4700 plasmid were determined by quantifying the number of splenocytes secreting IFN-γ in response to antigen-specific stimulation as measured by ELISPOT assay. The VR4700 plasmid encodes the influenza A/PR/8/34 nucleoprotein (NP) in the VR1055 backbone which is described in U.S. Pat. No. 6,586,409 and is incorporated herein by reference in its entirety. Briefly, ImmunoSpot plates (Millipore, Billerica, Mass.) were coated with rat anti-mouse IFN-γ monoclonal antibody (BD Pharmingen, San Diego, Calif.) and blocked with RPMI-1640 medium containing 10% fetal bovine serum (FBS, defined, Hyclone, Logan, Utah). Splenocyte suspensions were prepared from individual vaccinated mice and seeded in triplicate or quadruplicate wells of ImmunoSpot plates at densities ranging from 1×10⁵ to 1×10⁶ cells/well in RPMI-1640 stimulation medium containing 25 mM HEPES buffer and L-glutamine and supplemented with 10% FBS, 55 μM P-mercaptoethanol, 100 U/mL of penicillin G sodium salt, and 100 μg/mL of streptomycin sulfate (Invitrogen, Carlsbad, Calif.) and either 1 μg/mL of class I-restricted NP peptide (TYQRTRALV) or 20 μg/mL of recombinant NP protein (Imgenex, San Diego, Calif.). For CD8 T cell ELISPOT assays, the stimulation medium also contained 1 U/mL of recombinant murine IL-2. Control wells contained splenocytes incubated in medium with or without IL-2 (no antigen). After 20 hours, captured IFN-γ was detected by the sequential addition of biotin-labeled rat anti-mouse IFN-γ monoclonal antibody (BD pharmingen) and horseradish peroxidase-labeled avidin D (Vector Labs, Burlingame, Calif.). Spots produced by the conversion of the colorimetric substrate, 3-amino-9-ethylcarbazole (AEC, Vector Labs), were quantified by an ImmunoSpot Analyzer (Cellular Technology Ltd., Cleveland, Ohio). Data are presented as the number of antigen-specific Spot Forming Units (SFU) per million splenocytes. SFU counts were adjusted for background by substracting the number of spots in wells containing splenocytes in medium alone.

Anti-NP ELISA

Antibody response to NP expressed by VR4700 was determined by ELISA assay. Ninety-six well plates (Corning Incorporated, Cat. No. 3690, Corning, N.Y.) were coated with 71 ng/well of influenza A/PR/8/34 nucleoprotein (NP) purified from recombinant baculoviral extracts in 100 μl BBS (89 mM Boric Acid +90 mM NaCl +234 mM NaOH, pH 8.3). The plates were stored overnight at 4° C. and the wells washed twice with BBST (BBS supplemented with 0.05% Tween 20, vol/vol). The wells were then incubated for 90 minutes with BB (BBS supplemented with 5% nonfat milk, wt/vol) and washed twice with BBST again. Two-fold serial dilutions of mouse serum in BB, starting at 1:10, were made in successive wells and the solutions were incubated for 2 hours at room temperature. Wells were then rinsed four times with BBST. Sera from mice hyperimmunized with VR4700 NP plasmid were used as a positive control and pre-immune sera from mice were used as negative controls.

To detect NP-specific antibodies, alkaline phosphatase conjugated goat anti-mouse IgG-Fc (Jackson ImmunoResearch Laboratories, Cat. No. 115-055-008, West Grove, Pa.) diluted 1:5000 in BBS was added at 50 μl/well and the plates were incubated at room temperature for 2 hours. After 4 washings in BBST, 50 μl of substrate (1 mg/ml p-nitrophenyl phosphate, Calbiochem Cat. No. 4876 in 50 mM sodium bicarbonate buffer, pH 9.8 and 1 mM MgCl₂) was incubated for 90 min at room temperature and absorbance readings were performed at 405 nm. The titer of the sera was determined by using the reciprocal of the last dilution yielding an absorbance twice above background established using pre-immune serum diluted 1:20.

RT-PCR

RT-PCR was used to measure mRNA expression from plasmid VR4700 after in vitro transfection. VM92 murine cells (24 well plate format) were transfected with formulations with or without DMRIE:DOPE (DM:DP) transfection facilitating agent (1:1 mass ratio). A vial containing 0.48 mg DMRIE and 0.56 mg DOPE as a lipid film was reconstituted with 0.5 ml of PBS and vortexed at high speed to resuspend the lipid film. 1.2 μl of lipid was then added to 0.6 ml of the 2 μg/ml DNA test formulation and vortexed at medium speed for 15 seconds. The solution was then incubated at room temperature for 15 minutes. The pDNA/lipid complex was then diluted with 0.6 μl of Opti-MEM media. Formulations that do not require the addition of DM:DP were diluted to 2 μg/ml DNA with PBS, then a 0.6 ml aliquot was diluted with 0.6 μl of Opti-MEM media. The cells were transfected by removing the plating medium (RPMI 1640/10% FBS) from each well and replacing it with the 250 μl of the transfection solutions. Each formulation was tested in triplicate. The cells were incubated for 4 hours after which they were supplemented with medium. At 24-hours post transfection, cells were harvested, lysed, and total RNA isolated using a commercial RNA extraction kit. Individual preparations of purified total RNA were quantified by absorbance measurements using a spectrophotometer with a 260 m light source. Mass equivalents of total RNA were added to a commercial RT-PCR master mix along with commercially obtained application specific PCR primers and fluorescent probes. The 5′ forward primer (RM0014c) used was designed to span the intron in the expression plasmid thereby ensuring that only spliced messenger RNA could serve as a template for PCR amplification. A 3′ reverse primer (RM0228) hybridized to a region specific for each gene. During the RT step, reverse transcription of target RNA produced corresponding complementary DNA (cDNA) sequences. During the subsequent PCR, the initial concentration of target cDNA was quantified by amplifying it to a detectable level.

| | |
|---|---|
| TPROBE 04i - Probe: | |
| 5′CATTGCATCCATGATTGCTTCACAGCGTCC 3′ | (SEQ ID NO:1) |
| RM0014c - Forward Primer: | |
| 5′-CCGTGCCAAGAGTGACTCACC 3′ | (SEQ ID NO:2) |
| RM0228 - Reverse Primer: | |
| 5′CTCTAGCGCTGGGCGAAAC 3′ | (SEQ ID NO:3) |

The PCR reaction exploits the 5′ nuclease activity of AmpliTaq Gold® DNA Polymerase to cleave the TaqMan® probe during PCR. The TaqMan probe (TPROBE 04i) contains a reporter dye at the 5′ end of the probe and a quencher dye at the 3′ end of the probe. During the reaction, cleavage of the probe separates the reporter dye and the quencher dye, which results in increased fluorescence of the reporter. The resulting fluorescence emission between 500 m and 660 nm is collected from each well by the ABI Prism 7900HT Sequence Detection System, with a complete collection of data from all wells approximately once every 7-10 seconds.

The threshold cycle (Ct), for a given amplification curve, occurs at the point that the fluorescent signal grows beyond an empirically determined value, known as the threshold setting. It is at the threshold setting that the linear portion of the sigmoidal fluorescence intensity curve, characteristic of an actively progression polymerase chain reaction, can be readily differentiated from the background noise. The Ct represents a detection threshold for the 7900HT instrument and is dependent on two factors: the starting template copy number and the efficiency of DNA amplification. Since one master mix is used, the efficiency of amplification should be the same from well to well. Therefore, the Ct value is directly dependent on the starting RNA concentration.

### Example 1

Formation of Cell Delivery Particles Using Thermal Cycling Method

This example describes the interactions of poloxamer CRL-1005 and DNA with BAK C₁₂, BAK C₁₄, BAK C₁₆ and BAK C₁₈ to form cell delivery particles using the thermal cycling methods described in U.S. Published Patent Applications 2004/0162256 A1 and 2004/0209241 A1.

Stock solutions of benzyldimethyldodecylammonium chloride (BAK C₁₂, Fluka Chemical Corp., Milwaukee, Wis., cat #53233), benzyldimethyltetradecylammonium chloride (BAK C₁₄, TCI America, Portland, Oreg., cat #A0208), benzyldimethylhexadecyllammonium chloride (BAK C₁₆, TCI America, Portland, Oreg., cat #B0237) and benzyldimethyloctadecylammonium chloride (BAK C₁₈, TCI America, Portland, Oreg., cat #B1297) were made in PBS. The solubility of BAK C₁₂, C₁₄ and C₁₆ at 25° C. and 45° C. were recorded. See Table 2.

TABLE 2
Dissolution characteristics of BAK homologs

| | Concentration in | Dissolution | Precipitates at |
|---|---|---|---|
| Homolog | PBS (mM) | Temperature (° C.) | 25° C.? |
| C₁₄ | 5 | 25 | N |
| C₁₆ | 5 | 45 | Y |
| C₁₆ | 2 | 45 | Y |
| C₁₈ | 5 | 45 | Y |
| C₁₈ | 2 | 45 | Y |
| C₁₈ | 1 | 45 | Y |

The following solutions were then made using the thermal cycling method, without filtration, as described in FIG. 1 and U.S. Published Patent Applications 2004/0162256 A1 and 2004/0209241 A1.

1. 7.5 mg CRL-1005 in PBS

2. 7.5 mg CRL-1005+0.05 mM BAK C₁₄ in PBS

3. 7.5 mg CRL-1005+0.10 mM BAK C₁₄ in PBS

4. 7.5 mg CRL-1005+0.15 mM BAK C₁₄ in PBS

5. 7.5 mg CRL-1005+0.30 mM BAK C₁₄ in PBS

6. 7.5 mg CRL-1005+0.45 mM BAK C₁₄ in PBS

7. 7.5 mg CRL-1005+0.60 mM BAK C₁₄ in PBS

The size of the particles produced was determined using photon correlation spectroscopy. See Table 3. The particle size as reported herein refers to the mean Z average diameter also known as the hydrodynamic diameter. The surface charge of the particles was also determined using micro-electrophoresis. See Table 3.

TABLE 3

| BAK C₁₄ | Particle size | Polydispersity of particle | Surface |
|---|---|---|---|
| concentration mM | (nm) | size distribution | charge (mV) |
| 0 | 838 | 0.23 | −1.3 |
| 0.05 | 1588 | 0.13 | −0.5 |
| 0.10 | 858 | 0.6 | −1.5 |
| 0.15 | 405 | 0.45 | −1 |
| 0.30 | 207 | 0.05 | 0.8 |
| 0.45 | 222 | 0.09 | 2.8 |
| 0.60 | 207 | 0.13 | 3.9 |

The procedure was repeated for the BAK C₁₆ homolog (a 2 mM stock solution was warmed to 45° C. to dissolve any solids present prior to use). The size of the particles produced was determined using photon correlation spectroscopy and the surface charge of the particles was also determined using micro-electrophoresis. See Table 4.

TABLE 4

| BAK C₁₆ | Particle size | Polydispersity of particle | Surface |
|---|---|---|---|
| concentration mM | (nm) | size distribution | charge (mV) |
| 0 | 838 | 0.23 | −1.3 |
| 0.05 | 496 | 0.25 | 0.8 |
| 0.10 | 459 | 0.3 | 2.3 |
| 0.15 | 451 | 0.25 | 3.5 |
| 0.30 | 471 | 0.27 | 3.4 |
| 0.45 | 587 | 0.18 | 3.5 |
| 0.60 | 408 | 0.2 | 7.7 |

The procedure was repeated for the BAK C₁₈ homolog. Although there were visible particulates in these formulations, the particle size and surface charge of these formulations were measured. See Table 5.

TABLE 5

| BAK C₁₈ | Particle size | Polydispersity of particle | Surface |
|---|---|---|---|
| concentration mM | (nm) | size distribution | charge (mV) |
| 0 | 838 | 0.23 | −1.3 |
| 0.05 | 1400 | 0.36 | 3.5 |
| 0.10 | 1069 | 0.91 | 3.7 |
| 0.15 | 778 | 0.73 | 5.9 |
| 0.30 | 760 | 0.59 | 15.2 |
| 0.45 | 1188 | 0.66 | 15.3 |
| 0.60 | 657 | 0.39 | 17.6 |

The following cell delivery particles were made using BAK C₁₂ and the thermal cycling method without filtration, as described in FIG. 1 and U.S. Published Patent Applications 2004/0162256 A1 and 2004/0209241 A1.

1. 7.5 mg CRL-1005 in PBS

2. 7.5 mg CRL-1005+0.30 M BAK C₁₂ in PBS

3. 7.5 mg CRL-1005+0.60 M BAK C₁₂ in PBS

The size of the particles produced was determined using photon correlation spectroscopy and the surface charge of the particles was also determined using micro-electrophoresis. See Table 6.

TABLE 6

| BAK C₁₂ | Particle size | Polydispersity of particle | Surface |
|---|---|---|---|
| concentration mM | (nm) | size distribution | charge (mV) |
| 0 | 838 | 0.23 | −1.1 |
| 0.30 | 927 | 0.28 | +1.2 |
| 0.60 | 1494 | 0.79 | +1.5 |

The following cell delivery particles were made using the thermal cycling method with filtration depicted in FIG. 2 and U.S. Published Patent Applications 2004/0162256 A1 and 2004/0209241 A1.

1. 7.5 mg CRL-1005+0.10 mM BAK C₁₄+5.0 mg/ml DNA in PBS

2. 7.5 mg CRL-1005+0.15 mM BAK C₁₄+5.0 mg/ml DNA in PBS

3. 7.5 mg CRL-1005+0.30 mM BAK C₁₄+5.0 mg/ml DNA in PBS

4. 7.5 mg CRL-1005+0.45 mM BAK C₁₄+5.0 mg/ml DNA in PBS

5. 7.5 mg CRL-1005+0.60 mM BAK C₁₄+5.0 mg/ml DNA in PBS

The size of the particles produced was determined using photon correlation spectroscopy and the surface charge of the particles was also determined using micro-electrophoresis. See Table 7.

TABLE 7

| BAK C₁₄ | Particle size | Polydispersity of particle | Surface |
|---|---|---|---|
| concentration mM | (nm) | size distribution | charge (mV) |
| 0.10 | 436 | 0.36 | −0.9 |
| 0.15 | 441 | 0.35 | −1.6 |
| 0.30 | 295 | 0.04 | −0.3 |
| 0.45 | 256 | 0.07 | −1.2 |
| 0.60 | 436 | 0.42 | −4.9 |

The following cell delivery particles were made using the thermal cycling method with filtration, described in FIG. 2 and U.S. Published Patent Applications 2004/0162256 A1 and 2004/0209241 A1.

1. 7.5 mg CRL-1005+0.05 mM BAK C₁₆+5.0 mg/ml DNA in PBS

2. 7.5 mg CRL-1005+0.10 mM BAK C₁₆+5.0 mg/ml DNA in PBS

3. 7.5 mg CRL-1005+0.30 mM BAK C₁₆+5.0 mg/ml DNA in PBS

4. 7.5 mg CRL-1005+0.45 mM BAK C₁₆+5.0 mg/ml DNA in PBS

5. 7.5 mg CRL-1005+0.60 mM BAK C₁₆+5.0 mg/ml DNA in PBS

The size of the particles produced was determined using photon correlation spectroscopy and the surface charge of the particles was also determined using micro-electrophoresis. See Table 8. Above a concentration of 0.30 mM BAK C₁₆, DNA precipitation was observed below the cloud point of the poloxamer and visible precipitates could be seen in the formulation at room temperature.

TABLE 8

| BAK C₁₆ | Particle size | Polydispersity of particle | Surface |
|---|---|---|---|
| concentration mM | (nm) | size distribution | charge (mV) |
| 0.05 | 814 | 0.47 | −0.5 |
| 0.10 | 541 | 0.61 | 0.9 |
| 0.30 | 841 | 0.4 | −2 |
| 0.45 | 2370 | 1 | −6.2 |
| 0.60 | 6054 | 1 | −1.6 |

This experiment demonstrates that BAK C₁₂ and BAK C₁₈ do not form cell delivery particles with CRL-1005 and DNA using the thermal cycling method described in U.S. Published Patent Applications 2004/0162256 A1 and 2004/0209241 A1.

### Example 2

The following example describes the change in particle size and surface charge when poloxamer solutions with or without cationic lipids are subject to high pressure homogenization.

Particles of poloxamers CRL-8300 and CRL-1005 at a concentration of 7.5 mg/ml each in PBS (30 ml) were prepared by homogenization in an EmulsiFlex-C50 high pressure homogenizer at 15,000 psi and 15° C. The particles were collected in a 50 ml conical tube after 10 passes through the adjustable homogenizing valve. The size of the particles produced was determined using photon correlation spectroscopy (See Table 9) and the surface charge of the particles was also determined using micro-electrophoresis. See Table 10.

TABLE 9

| | Particle size nm | Particle size nm |
|---|---|---|
| | (Polydispersity) | (Polydispersity) |
| Solution | before homogenization | after homogenization |
| CRL-8300 @ 7.5 mg/ml | 2,353 (0.84) | 200 (0.23) |
| in PBS | | |
| CRL-1005 @ 7.5 mg/ml | 1,752 (0.25) | 228 (0.4) |
| in PBS | | |

TABLE 10

| | Surface charge mV | Surface charge mV |
|---|---|---|
| Solution | before homogenization | after homogenization |
| CRL-8300 @ 7.5 mg/ml | −1.4 | Not determined |
| in PBS | | |
| CRL-1005 @ 7.5 mg/ml | +0.3 | −4.0 |
| in PBS | | |

The process was then repeated with 30 ml of CRL-8300 at a concentration of 7.5 mg/ml (in PBS) in the presence of 0.3 mM BAK (made from a 50% solution of BTC 50 NF). In another experiment, BAK was replaced with 0.01 mM DMRIE. Both formulations were homogenized in an EmulsiFlex-C50 high pressure homogenizer at 15,000 psi and 15° C. Each solution was collected in a 50 ml conical tube after 10 passes through the adjustable homogenizing valve. The size of the particles produced was determined using photon correlation spectroscopy and the surface charge of the particles was also determined using micro-electrophoresis. See Table 11.

TABLE 11

| | | Particle size nm |
|---|---|---|
| Solution | Surface charge (mV) | (polydispersity) |
| CRL-8300 @ 7.5 mg/ml | +3.3 | 203 (0.24) |
| 0.3 mM BTC 50 NF | | |
| in PBS | | |
| CRL-8300 @ 7.5 mg/ml | +3.8 | 207 (0.20) |
| 0.01 mM DMRIE | | |
| in PBS | | |

The experiments in this example demonstrate that when certain poloxamer solutions are subjected to high pressure homogenization, small uniform particles are produced. Furthermore, when a cationic lipid is present the surface charge, which is otherwise near neutral, becomes positive.

### Example 3

This example describes the change in particle size and surface charge when poloxamer and DMRIE solutions are subject to high pressure homogenization. The change in particle size and surface charge of the homogenized particles after the addition of DNA is also described

Stock solutions of 15 mg/ml CRL-8300 in 2× PBS and 0.2 or 2.0 mM DMRIE in sterile water for injection were made. 15 ml of the poloxamer solution and 15 ml of the 0.2 mM lipid solution were then mixed in a 50 ml conical tube by gentle inversion at room temperature. The size of the particles produced was determined using photon correlation spectroscopy and the surface charge of the particles was also determined using micro-electrophoresis. See Table 12.

The solution was then homogenized in an EmulsiFlex-C50 high pressure homogenizer at 15,000 psi and 15° C. for 10 passes through the adjustable homogenizing valve and collected in a 50 ml conical tube. The size of the particles produced was determined using photon correlation spectroscopy and the surface charge of the particles was also determined using micro-electrophoresis. See Table 12. The solution was then homogenized for a further 10 passes, collected in a 50 ml conical tube and the particle size and surface charge analysis repeated. See Table 12.

15 ml of the poloxamer solution and 15 ml of the 2 mM lipid solution were then mixed in a 50 ml conical tube by gentle inversion at room temperature. The size of the particles produced was determined using photon correlation spectroscopy and the surface charge of the particles was also determined using micro-electrophoresis. See Table 12. The solution was then homogenized in an EmulsiFlex-C50 high pressure homogenizer at 15,000 psi and 15° C. for 10 passes through the adjustable homogenizing valve and collected in a 50 ml conical tube. The size of the particles produced was determined using photon correlation spectroscopy and the surface charge of the particles was also determined using micro-electrophoresis. See Table 12.

TABLE 12

| | Particle size | | Particle size | | Particle size | |
|---|---|---|---|---|---|---|
| | (nm) before | Surface charge | (nm) after ×10 | Surface charge | (nm) after ×20 | Surface charge |
| | homogenization | (mV) before | homogenization | (mV) after ×10 | homogenization | (mV) after ×20 |
| Formulation | (polydispersity) | homogenization | (polydispersity) | homogenization | (polydispersity) | homogenization |
| 7.5 mg/ml | 13,637 (1.0) | +1.0 | 200 (0.26) | +17.5 | 196 (0.18) | +18.2 |
| CRL-8300 | | | | | | |
| 0.1 mM | | | | | | |
| DMRIE in | | | | | | |
| PBS | | | | | | |
| 7.5 mg/ml | 19,174 (1.0) | +1.6 | 202 (0.48) | 30.0 | Not | Not |
| CRL-8300 | | | | | determined | determined |
| 1.0 mM | | | | | | |
| DMRIE in | | | | | | |
| PBS | | | | | | |

The following formulations:
1. 7.5 mg/mil CRL-8300+0.1 mM DMRIE 20 passes through homogenizer at 15,000 psi, 15° C.
2. 7.5 mg/mil CRL-8300+1.0 mM DMRIE 10 passes through homogenizer at 15,000 psi, 15° C.

were left at room temperature for 12 hours and the particle size measured using photon correlation spectroscopy showing stability of the particles over time. See Table 13. 2 ml samples of the two solutions were also cooled below the cloud point of the poloxamer and then warmed to room temperature and the size of the particles produced was determined using photon correlation spectroscopy (See Table 14) and the surface charge of the particles was also determined using micro-electrophoresis. See Table 14.

TABLE 13

| | Particle size (nm) after | Particle size (nm) after |
|---|---|---|
| | homogenization T = 0 | homogenization |
| Formulation | (polydispersity) | T = 12 (polydispersity) |
| 7.5 mg/ml CRL-8300 | 196 (0.18) | 204 (0.19) |
| 0.1 mM DMRIE in PBS | | |
| 7.5 mg/ml CRL-8300 | 202 (0.48) | 213 (0.35) |
| 1.0 mM DMRIE in PBS | | |

TABLE 14

| | Particle size (nm) | | Particle size (nm) | |
|---|---|---|---|---|
| | after | Surface charge | after 1x thermal | Surface charge |
| | homogenization | (mV) after | cycle | (mV) after 1x |
| Formulation | (polydispersity) | homogenization | (polydispersity) | thermal cycle |
| 7.5 mg/ml CRL-8300 | 196 (0.18) | +18.2 | 327 (0.33) | +3.4 |
| 0.1 mM DMRIE in PBS | | | | |
| 7.5 mg/ml CRL-8300 | 202 (0.48) | +30.0 | 491 (0.46) | +19.8 |
| 1.0 mM DMRIE in PBS | | | | |

Plasmid DNA was then added to formulation #2 (7.5 mg/ml CRL-8300+1.0 mM DMRIE, 10 passes through homogenizer at 15,000 psi, 15° C.). 1 ml of the poloxamer lipid solution was placed in a 2 ml Eppendorf tube and 10 μl of a 6.58 mg/ml plasmid solution was added using a 20 μl pipette and the tube was mixed by gentle inversion four times. The charge ratio (+/−, DNA phosphate: cationic lipid) of the resulting solution was 0.2. The millimolar concentration of plamid DNA phosphate is calculated by dividing the plasmid DNA concentration (in mg/ml) by 330, the average nucleotide molecular mass. Since DMRIE contains a single point charge, the millimolar ratio of plasmid DNA phosphate can be compared to the millimolar concentratrion of DMRIE to calculate the −/+charge ratio. The solution was left to incubate at room temperature for 30 minutes and the particle size and surface charge were measured. See Table 15. The process was repeated with a second sample at a charge ratio of 2.0 (100 μl of 6.58 mg/ml DNA) and the particle size and surface charge were measured. See Table 15.

TABLE 15

| | Particle size (nm) | |
|---|---|---|
| | after 30 min. | Surface charge (mV) |
| | incubation at room temp. | after 30 min. incubation |
| Formulation | (polydispersity) | at room temp. |
| 7.5 mg/ml CRL- | 202 (0.48) | +30.0 |
| 8300 | | |
| 1.0 mM DMRIE in | | |
| PBS | | |
| 7.5 mg/ml CRL- | 229 (019) | +31.7 |
| 8300 | | |
| 1.0 mM DMRIE | | |
| 66 μg DNA, | | |
| charge ratio 0.2 | | |
| 7.5 mg/ml CRL- | 441 (0.74) | −42.4 |
| 8300 | | |
| 1 mM DMRIE | | |
| 658 μg DNA, | | |
| charge ratio 2.0 | | |

The experiments of this example demonstrate that when certain poloxamer solutions are subjected to high pressure homogenization in the presence of the cationic lipid DMRIE, small uniform particles are produced with a positive surface charge. When DNA is incubated with these particles, a stable cell delivery particle is produced that has a positive surface charge in the presence of a molar excess of DMRIE and a negative surface charge when using a molar excess of DNA.

### Example 4

This example describes the change in particle size and surface charge when poloxamer and DMRIE solutions are subject to high pressure homogenization. The changes in particle size and surface charge of the homogenized particles after the addition of DNA is also described.

Stock solutions of 15 mg/ml CRL-8300 in 2× PBS and 0.2 or 2.0 mM DMRIE in sterile water for injection were made. 15 ml of the poloxamer solution and 15 ml of the 2.0 mM lipid solution were then mixed in a 50 ml conical tube by gentle inversion at room temperature. The solution was then homogenized in an EmulsiFlex-C50 high pressure homogenizer at 15,000 psi and 15° C. for 10 passes through the adjustable homogenizing valve and collected in a 50 ml conical tube. The size of the particles produced was determined using photon correlation spectroscopy. See Table 16. The solution was then homogenized for a further 5 passes, collected in a 50 ml conical tube and the particle size and surface charge analysis repeated. See Table 16.

15 ml of the poloxamer (15 mg/ml CRL-8300) solution and 15 ml of a 4.0 mM DMRIE solution in sterile water were mixed in a 50 ml conical tube by gentle inversion at room temperature. The solution was then homogenized in an EmulsiFlex-C50 high pressure homogenizer at 15,000 psi and 15° C. for 15 passes through the adjustable homogenizing valve and collected in a 50 ml conical tube. The size of the particles produced was determined using photon correlation spectroscopy and the surface charge of the particles was also determined using micro-electrophoresis. See Table 16. 15 ml of the poloxamer (15 mg/ml CRL-8300) solution and 15 ml of 6.0 mM lipid solution were then mixed in a 50 ml conical tube by gentle inversion at room temperature. The solution was then homogenized in an EmulsiFlex-C50 high pressure homogenizer at 15,000 psi and 15° C. for 15 passes through the adjustable homogenizing valve and collected in a 50 ml conical tube. The size of the particles produced was determined using photon correlation spectroscopy and the surface charge of the particles was also determined using micro-electrophoresis. See Table 16.

TABLE 16

| | Particle size (nm) | | Particle size (nm) | |
|---|---|---|---|---|
| | after x10 | Surface charge | after x15 | Surface charge |
| | homogenization | (mV) after x10 | homogenization | (mV) after x15 |
| Formulation | (polydispersity) | homogenization | (polydispersity) | homogenization |
| 7.5 mg/ml CRL-8300 | 162 (0.23) | Not determined | 165 (0.16) | +32.3 |
| 1.0 mM DMRIE in PBS | | | | |
| 7.5 mg/ml CRL-8300 | Not determined | Not determined | 169 (0.15) | +36.5 |
| 2.0 mM DMRIE in PBS | | | | |
| 7.5 mg/ml CRL-8300 | Not determined | Not determined | 157 (0.20) | +35.8 |
| 3.0 mM DMRIE in PBS | | | | |

VR4700 plasmid DNA (6.58 mg/ml) was then added to 1 ml of each formulation, described in Table 16, in a eppendorf tube, via pipette, and the solution mixed by inversion five times. DNA:cationic lipid charge ratios of 0.2, 0.6 and 2.0 were made, incubated at room temperature for 30 minutes and the solutions visually inspected. See Table 17. Those formulations without visible particulates were then physically characterized. The particle size and zeta potential of particles produced containing DNA:cationic lipid charge ratios of 0.2, 0.6 and 2.0 were measured. See Table 18 (particle size) and Table 19 (zeta potential).

TABLE 17

| | Charge | Charge | Charge |
|---|---|---|---|
| Formulation | ratio 0.2 | ratio 0.6 | ratio 2.0 |
| 7.5 mg/ml CRL-8300 | Milky white | Milky white | Milky white |
| 1.0 mM DMRIE in PBS | suspension | suspension | suspension |
| 7.5 mg/ml CRL-8300 | Milky white | Visible | Visible |
| 2.0 mM DMRIE in PBS | suspension | particulates | particulates |
| 7.5 mg/ml CRL-8300 | Milky white | Visible | Visible |
| 3.0 mM DMRIE in PBS | suspension | particulates | particulates |

TABLE 18

| | Charge | Charge | Charge |
|---|---|---|---|
| Formulation | ratio 0.2 | ratio 0.6 | ratio 2.0 |
| 7.5 mg/ml CRL-8300 | 215 (0.19) | 423 (0.28) | 406 (0.46) |
| 1.0 mM DMRIE in PBS | | | |
| 7.5 mg/ml CRL-8300 | 246 (0.20) | Not | Not |
| 2.0 mM DMRIE in PBS | | determined | determined |
| 7.5 mg/ml CRL-8300 | 223 (0.26) | Not | Not |
| 3.0 mM DMRIE in PBS | | determined | determined |

TABLE 19

| | Charge | Charge | Charge |
|---|---|---|---|
| Formulation | ratio 0.2 | ratio 0.6 | ratio 2.0 |
| 7.5 mg/ml CRL-8300 | +35.8 | +35.3 | −38.0 |
| 1.0 mM DMRIE in PBS | | | |
| 7.5 mg/ml CRL-8300 | +39.5 | Not | Not |
| 2.0 mM DMRIE in PBS | | determined | determined |
| 7.5 mg/ml CRL-8300 | +40.4 | Not | Not |
| 3.0 mM DMRIE in PBS | | determined | determined |

The experiments of this example demonstrate that when certain poloxamer solutions are subjected to high pressure homogenization in the presence of the cationic lipid DMRIE, small uniform particles are produced with a positive surface charge. When DNA is incubated with these particles, a stable cell delivery particle is produced that has a positive surface charge in the presence of a molar excess of DMRIE and a negative surface charge when using a molar excess of DNA.

### Example 5

This example also describes the change in particle size and surface charge when certain poloxamer and DMRIE solutions are subject to high pressure homogenization. The changes in particle size and surface charge of the homogenized particles after the addition of DNA is also described.

A 50 mg/ml CRL-1005 solution in 2× PBS was made as described in the general experimental section. This stock solution was then diluted with 2× PBS to give a 15 mg/ml and 40 mg/ml solution of CRL-1005. A 2 mM, 4 mM and 6 mM, solution of DMRIE was also made in sterile water for injection.

15 ml of the 15 mg/ml poloxamer stock solution and 15 ml of the 2.0 mM lipid solution were mixed in a 50 ml conical tube by gentle inversion at room temperature. The size of the particles produced was determined using photon correlation spectroscopy. See Table 20. The solution was then homogenized in an EmulsiFlex-C50 high pressure homogenizer at 15,000 psi and 15° C. for 15 passes through the adjustable homogenizing valve and collected in a 50 ml conical tube. The size of the particles produced was determined using photon correlation spectroscopy. See Table 20. The solution was then homogenized for a further 5 passes, collected in a 50 ml conical tube and the particle size analysis repeated. See Table 20. The process was repeated for at total of 25 and 30 passes through the homogenizer valve. See Table 20.

TABLE 20

| Passes through | | Polydispersity of particle |
|---|---|---|
| homogenizer valve | Particle size (nm) | size distribution |
| 15 | 874 | 0.974 |
| 20 | 209 | 0.355 |
| 25 | 188 | 0.298 |
| 30 | 193 | 0.274 |

15 ml of the 40 mg/ml poloxamer stock solution and 15 ml of the 4.0 mM lipid solution were mixed in a 50 ml conical tube by gentle inversion at room temperature. The solution was then homogenized in an EmulsiFlex-C50 high pressure homogenizer at 15,000 psi and 15° C. for 30 passes through the adjustable homogenizing valve and collected in a 50 ml conical tube. The size of the particles produced was determined using photon correlation spectroscopy and the surface charge of the particles was also determined using micro-electrophoresis. See Table 21. The particle size and surface charge analysis was repeated after storage at room temperature for 12 hours. See Table 21.

15 ml of the 50 mg/ml poloxamer stock solution and 15 ml of the 6.0 mM lipid solution were mixed in a 50 ml conical tube by gentle inversion at room temperature. The solution was then homogenized in an EmulsiFlex-C50 high pressure homogenizer at 15,000 psi and 15° C. for 30 passes through the adjustable homogenizing valve and collected in a 50 ml conical tube. The size of the particles produced was determined using photon correlation spectroscopy and the surface charge of the particles was also determined using micro-electrophoresis. See Table 21. The particle size and surface charge analysis was repeated after storage at room temperature for 12 hours. See Table 21.

TABLE 21

| | | Surface | | Surface |
|---|---|---|---|---|
| | Particle size nm | charge (mV) | Particle size nm | charge (mV) |
| Formulation | (polydispersity) T = 0 | T = 0 | (polydispersity) T = 12 h | T = 12 h |
| 7.5 mg/ml CRL-1005 | 193 (0.27) | +20.2 | Not determined | Not |
| 1.0 mM DMRIE in PBS | | | | determined |
| 20 mg/ml CRL-1005 | 208 (0.3) | +31.8 | 208 (0.3) | +33.7 |
| 2.0 mM DMRIE | | | | |
| in PBS | | | | |
| 25 mg/ml CRL-1005 | 209 (0.3) | +30.7 | 202 (0.26) | +31.6 |
| 3.0 mM DMRIE | | | | |
| in PBS | | | | |

1 ml of the solution described above was aliquoted, at room temperature, into 3 different 2 ml eppendorf tubes and VR4700 plasmid DNA was added at three different charge ratios:

1. 15.2 μl of 6.58 mg/ml DNA in PBS=100 μg or 0.1 charge ratio (−/+)

2. 30.4 μl of 6.58 mg/ml DNA in PBS=200 μg or 0.2 charge ratio (−/+)

3. 46.0 μl of 6.58 mg/ml DNA in PBS=300 μg or 0.3 charge ratio (−/+)

The solutions were then mixed by gentle inversion 5 times and incubated at room temperature for 30 minutes. The size of the particles produced was determined using photon correlation spectroscopy, the surface charge of the particles was also determined using micro-electrophoresis and the visual appearance was documented. See Table 22.

TABLE 22

| | Particle | | |
|---|---|---|---|
| | size nm | Surface | |
| Formulation | (polydispersity) | charge (mV) | Visual appearance |
| 25 mg/ml CRL-1005 | 224 (0.27) | +16.3 | Milky white |
| 3.0 mM DMRIE | | | solution |
| 100 μg DNA | | | |
| in PBS | | | |
| 25 mg/ml CRL-1005 | 252 (0.33) | +9.3 | Milky white |
| 3.0 mM DMRIE | | | solution |
| 200 μg DNA | | | |
| in PBS | | | |
| 25 mg/ml CRL-1005 | 299 (0.36) | +12.3 | Milky white |
| 3.0 mM DMRIE | | | solution + visible |
| 300 μg DNA | | | aggregates |
| in PBS | | | |

The experiments in the example demonstrate that when poloxamer solutions are subjected to high pressure homogenization in the presence of the cationic lipid DMRIE, small uniform particles are produced with a positive surface charge. When DNA is incubated with these particles, a stable cell delivery particle is produced that has a positive surface charge in the presence of a molar excess of DMRIE.

### Example 6

This example describes the change in particle size and surface charge when certain poloxamer and DMRIE solutions are subject to high pressure homogenization. The sterile filtration of the homogenized particles was also tested.

272 mg of DMRIE was dissolved in 71 ml of sterile water for injection and was homogenized and extruded in an EmulsiFlex-C50 high pressure homogenizer fitted with an optional filter/extruder (F) down stream from the homogenizer valve. See FIG. 4. The solution was processed at 10,000 psi and 15° C. for 5 passes through the adjustable homogenizing valve. The extruder was fitted with three 50 nm pore size filter membranes and the solution was collected after processing in a 50 ml conical tube. The size of the resulting particles were measured by photon correlation spectroscopy after 2 and 12 hours storage at 4° C. See Table 23.

TABLE 23

| | Particle size nm | Particle size nm | Particle size nm |
|---|---|---|---|
| | (polydispersity) | (polydispersity) | (polydispersity) |
| Formulation | T = 2 h | T = 12 h | T = 16 days |
| 6 mM DMRIE | 56 (0.21) | 62 (0.13) | 60 (0.14) |
| liposome's | | | |

11.5 ml of a 30 mg/ml CRL-1005 solution in 2× PBS and 11.5 ml of the 6.0 mM DMRIE liposome solution were mixed in a 50 ml conical tube by gentle inversion at room temperature. The size of the particles produced was determined using photon correlation spectroscopy and the surface charge of the particles was also determined using micro-electrophoresis. See Table 24. The solution was then homogenized in an EmulsiFlex-C50 high pressure homogenizer at 15,000 psi and 15° C. for 30 passes through the adjustable homogenizing valve and collected in a 50 ml conical tube. The size of the particles produced was determined using photon correlation spectroscopy and the surface charge of the particles was also determined using micro-electrophoresis. See Table 24. A 3 ml sample of the solution post-homogenization at room temperature was then drawn up into a 5 ml syringe and passed through a 0.2 μm posidyne filter. The particle size and surface charge of the particle were then determined post-filtration. See Table 24.

TABLE 24

| | Particle size nm | Surface charge |
|---|---|---|
| Formulation | (polydispersity) | (mV) |
| 15 mg/ml CRL-1005 | 904 (0.92) | +11.4 |
| 3.0 mM DMRIE in PBS | | |
| Pre-homogenization | | |
| 15 mg/ml CRL-1005 | 192 (0.33) | +39.0 |
| 3.0 mM DMRIE in PBS | | |
| Post-homogenization | | |
| 15 mg/ml CRL-1005 | 279 (0.20) | +44.2 |
| 3.0 mM DMRIE in PBS post 0.2 μm | | |
| filtration | | |

The experiments in this example demonstrate that when certain poloxamer solutions are subjected to high pressure homogenization in the presence of DMRIE, small uniform particles are produced with a positive surface charge. These particles can then be subjected to conditions for filter sterilization.

### Example 7

This example describes the change in particle size and surface charge when poloxamer solutions and VC1052 are subject to high pressure homogenization.

268 mg of VC1052 was dissolved in 74 ml of sterile water for injection (6 mM stock) and was homogenized and extruded in an EmulsiFlex-C50 high pressure homogenizer fitted with an optional filter/extruder down stream from the homogenizer valve. The solution was processed at 10,000 psi and 15° C. for 5 passes through the adjustable homogenizing valve. The extruder was fitted with three 50 nm pore size filter membranes and the solution was collected after processing in a 50 ml conical tube. The size of the particles after storage for 12 hours at 4° C. was measured by photon correlation spectroscopy. See Table 25.

TABLE 25

| | Particle size nm |
|---|---|
| Formulation | (polydispersity) T = 2 h |
| 6 mM VC1052 liposome's | 57 (0.38) |

A 30 mg/ml CRL-1005 solution in sterile water for injection was made as described in the methods section. 15 ml was then mixed with 15 ml of the 6.0 mM VC1052 lipid solution in a 50 ml conical tube by gentle inversion at room temperature. The size of the particles produced was determined using photon correlation spectroscopy and the surface charge of the particles was also determined using micro-electrophoresis. See Table 26. The solution was then homogenized in an EmulsiFlex-C50 high pressure homogenizer at 15,000 psi and 15° C. for 30 passes through the adjustable homogenizing valve and collected in a 50 ml conical tube. The size of the particles produced was determined using photon correlation spectroscopy and the surface charge of the particles was also determined using micro-electrophoresis. See Table 26.

TABLE 26

| | Particle | | Particle | |
|---|---|---|---|---|
| | size nm | Surface | size nm | Surface |
| | (polydispersity) | charge | (polydispersity) | charge |
| | Pre- | (mV) Pre- | Post- | (mV) Post- |
| | homogeni- | homogeni- | homogeni- | homogeni- |
| Formulation | zation | zation | zation | zation |
| 15 mg/ml | 1056 (0.67) | +43.1 | 198 (0.32) | +36.2 |
| CRL-1005 | | | | |
| 3.0 mM | | | | |
| VC1052 | | | | |
| in SWFI | | | | |

The experiments of this example demonstrate that when certain poloxamer solutions are subjected to high pressure homogenization in the presence of VC1052, small uniform particles are produced with a positive surface charge.

### Example 8

This experiment describes the change in particle size and surface charge when certain poloxamer and DMRIE solutions are subject to high pressure homogenization. The change in particle size and surface charge of the homogenized particles after the addition of DNA are also described. These formulations were then tested for activity in vitro and in vivo.

The size of the DMRIE particles made in Example 6 was measured after 16 days storage at 4° C., by photon correlation spectroscopy and was shown to be unchanged. See Table 23. A 15 mg/ml CRL-1005 solution in PBS was made as described in the methods section. Three preparations of CRL-1005+DMRIE particles in PBS were made by homogenization:

1. 7.5 mg/ml CRL-1005+0.1 mM DMRIE in PBS

15 ml of the poloxamer solution at 15 mg/ml and 15 ml of 0.2 mM DMRIE (6 mM stock diluted with sterile water for injection) were then mixed in a 50 ml conical tube by gentle inversion at room temperature.

2. 7.5 mg/ml CRL-1005+1.0 mM DMRIE in PBS

15 ml of the poloxamer solution at 15 mg/ml and 15 ml of 2.0 mM DMRIE (6 mM stock diluted with sterile water for injection) were then mixed in a 50 ml conical tube by gentle inversion at room temperature.

3. 7.5 mg/ml CRL-1005+2.0 mM DMRIE in PBS

15 ml of the poloxamer solution at 15 mg/ml and 15 ml of 4.0 mM DMRIE (6 mM stock diluted with sterile water for injection) were then mixed in a 50 ml conical tube by gentle inversion at room temperature.

The size of all particles produced was determined using photon correlation spectroscopy and the surface charge of the particles was also determined using micro-electrophoresis. See Table 27. The solution was then homogenized in an EmulsiFlex-C50 high pressure homogenizer at 15,000 psi and 15° C. for 30 passes through the adjustable homogenizing valve and collected in a 50 ml conical tube. The size of the particles produced was determined using photon correlation spectroscopy and the surface charge of the particles was also determined using micro-electrophoresis. See Table 27. Particle size and surface charge characterization was repeated after storage at room temperature for 41 days. See Table 27.

TABLE 27

| | | | | | | Surface |
|---|---|---|---|---|---|---|
| | | | | | | charge |
| | | Surface | | Surface | | (mV) |
| | | charge | | charge | Particle size nm | Post- |
| | Particle size nm | (mV) | Particle size nm | (mV) | (polydispersity) | homogen. |
| | (polydispersity) | Pre- | (polydispersity) | Post- | Post-homogen. | 41 days at |
| Formulation | Pre-homog. | homog. | Post-homogen. | homogen. | 41 days at RT | RT |
| 7.5 mg/ml | 875 (0.62) | +0.1 | 195 (0.08) | +4.3 | 201 (0.09) | +16.1 |
| CRL-1005 | | | | | | |
| 0.1 mM | | | | | | |
| DMRIE | | | | | | |
| in PBS | | | | | | |
| 7.5 mg/ml | 673 (0.66) | +5.7 | 192 (0.24) | +17.7 | 245 (0.18) | +40.6 |
| CRL-1005 | | | | | | |
| 1.0 mM | | | | | | |
| DMRIE | | | | | | |
| in PBS | | | | | | |
| 7.5 mg/ml | 1109 (0.62) | +10.5 | 185 (0.33) | +33.3 | 253 (0.24) | +38.3 |
| CRL-1005 | | | | | | |
| 2.0 mM | | | | | | |
| DMRIE | | | | | | |
| in PBS | | | | | | |

In Vivo Analysis of Formulations 1, 2, and 3

1.5 ml of formulations 1, 2 and 3 were placed into separate 2 ml glass vials and 24.2 μl of plasmid (VR4700) at 6.2 mg/ml was added with a 100 μl pipette. The vials were then sealed with a rubber stopper, the solution inside mixed by gentle inversion 4 times and incubated at room temperature for 30 minutes. Naked DNA, DNA, CRL-1005 and BAK or CRL-1005 02A (5 mg/ml DNA, 7.5 mg/ml CRL-1005, and 0.3 mM BAK) solutions were thermal cycled according to the method described in FIG. 2 and DM:DP (4:1) formulations were used as controls. The following groups were then tested:

1. 10 μg VR4700 (5 μg/50 μl/leg)

2. 10 μg VR4700+CRL-1005 02A

3. 10 μg VR4700+CRL/DM charge ratio 0.15 (formulation 3)

4. 10 μg VR4700+CRL/DM charge ratio 0.30 (formulation 2)

5. 10 μg VR4700+CRL/DM charge ratio 3.0 (formulation 1)

6. 10 μg VR4700+DM:DP (4:1)

BALB/c female mice (9/group, 54 mice total) were given bilateral intramuscular injections into the rectus femoris with a 5 μg dose of plasmid DNA in 50 μl per leg. Mice received injections on days 0, 20 and 48. Orbital sinus puncture (OSP) bleeds were taken on day 61 and splenocytes were harvested on days 62, 63 and 64. NP-specific antibodies were analyzed by ELISA (See Table 28) and NP-specific Th and Tc cells were analyzed by IFN-γ ELISPOT (See Tables 29 and 30) as described in the methods section.

TABLE 28

| | NP specific |
|---|---|
| Formulation | serum Ab titers (total IgG) |
| 10 μg VR 4700 (5 μg/50 μl/leg | 28,444 |
| 10 μg VR 4700 + CRL-1005 02A | 82,489 |
| 10 μg VR 4700 + CRL/DM charge ratio | <10 |
| 0.15 | |
| 10 μg VR 4700 + CRL/DM charge ratio | <10 |
| 0.3 | |
| 10 μg VR 4700 + CRL/DM charge ratio | 54,044 |
| 3.0 | |
| 10 μg VR 4700 + DM:DP, 4:1 | 88,178 |

TABLE 29

| | CD8⁺ IFN-γ ELISPOT |
|---|---|
| Formulation | (SFU/10⁶ cells) |
| 10 μg VR 4700 (5 μg/50 μl/leg | 87 |
| 10 μg VR 4700 + CRL-1005 02A | 202 |
| 10 μg VR 4700 + CRL/DM charge ratio 0.15 | 3 |
| 10 μg VR 4700 + CRL/DM charge ratio 0.3 | 13 |
| 10 μg VR 4700 + CRL/DM charge ratio 3.0 | 123 |
| 10 μg VR 4700 + DM:DP, 4:1 | 244 |

TABLE 30

| | CD4⁺ IFN-γ ELISPOT |
|---|---|
| Formulation | (SFU/10⁶ cells) |
| 10 μg VR 4700 (5 μg/50 μl/leg | 57 |
| 10 μg VR 4700 + CRL-1005 02A | 169 |
| 10 μg VR 4700 + CRL/DM charge ratio 0.15 | 0 |
| 10 μg VR 4700 + CRL/DM charge ratio 0.3 | 0 |
| 10 μg VR 4700 + CRL/DM charge ratio 3.0 | 61 |
| 10 μg VR 4700 + DM:DP, 4:1 | 331 |

In Vitro Analysis of Formulations 1, 2, and 3

1.5 ml of formulations 1, 2 and 3 were placed into separate 2 ml glass vials and 24.2 μl of plasmid (VR4700) at 6.2 mg/ml was added with a 100 μl pipette. The vials were then sealed with a rubber stopper, the solution inside mixed by gentle inversion 4 times and incubated at room temperature for 30 minutes. Naked DNA, DNA, CRL-1005 and BAK or CRL-1005 02A (5 mg/ml DNA, 7.5 mg/ml CRL-1005, and 0.3 mM BAK) thermal cycled according to FIG. 2 and VR4700+DM (charge ratio 0.15, 0.3 and 3.0) formulations were used as controls. The following groups were then tested in vitro:

1. VR4700 in PBS

2. VR4700+CRL-1005 02A

3. VR4700+CRL/DM charge ratio 0.15 (formulation 3)

4. VR4700+CRL/DM charge ratio 0.30 (formulation 2)

5. VR4700+CRL/DM charge ratio 3.0 (formulation 1)

6. VR4700+DM charge ratio 0.15

7. VR4700+DM charge ratio 0.30

8. VR4700+DM charge ratio 3.0

The in vitro expression of mRNA was then evaluated using RT-PCR (See Table 31) as described in the methods section.

TABLE 31

| Formulation | Cycles to threshold (Ct) |
|---|---|
| VR 4700* | 28.2 |
| VR 4700 | >40 |
| VR 4700 + CRL-1005 02A* | 26.7 |
| VR 4700 + CRL-1005 02A | >40 |
| VR 4700 + CRL/DM charge ratio 0.15 | 30.0 |
| VR 4700 + CRL/DM charge ratio 0.3 | 32.3 |
| VR 4700 + CRL/DM charge ratio 3.0 | >40 |
| VR 4700 + DM charge ratio 0.15 | 37.1 |
| VR 4700 + DM charge ratio 0.3 | 27.3 |
| VR 4700 + DM charge ratio 3.0 | 29.0 |

*DM:DP added as a Transfection facilitating agent

The experiments of this example demonstrate that when certain poloxamer solutions were subjected to high pressure homogenization in the presence of DMRIE, small uniform particles were produced with a positive surface charge. When DNA is incubated with these particles, a stable cell delivery particle is produced that has a positive surface charge in the presence of a molar excess of DMRIE and a negative surface charge using a molar excess of DNA. These cell delivery particle based formulations of DNA were biologically active in vivo and in vitro.

### Example 9

This example describes the changes in particle size and surface charge when poloxamer and DMRIE solutions are homogenization, lyophilized and then reconstituted.

269 mg of DMRIE was dissolved in 70 ml of sterile water for injection and was homogenized and extruded in an EmulsiFlex-C50 high pressure homogenizer fitted with an optional filter/extruder (F) down stream from the homogenizer valve. See FIG. 4. The solution was processed at 10,000 psi and 15° C. for 5 passes through the adjustable homogenizing valve. The extruder was fitted with three 50 m pore size filter membranes and the solution was collected after processing in a 50 ml conical tube. Particle size, as measured by photon correlation spectroscopy, was found to be 65 nm with a polydispersity of 0.21.

A 15 mg/ml CRL-1005 solution in 17% sucrose and a 18 mg/mL CRL-1005 solution in sterile water for injection were made as described in the general methods section. Two methods were then used to form complexes between the poloxamer and lipid.

Method 1

15 ml of the poloxamer solution at 15 mg/ml in 17% sucrose and 15 ml of a 2.0 mM DMRIE (6 mM stock diluted with sterile water for injection) were mixed in a 50 ml conical tube by gentle inversion at room temperature. The solution was then homogenized in an EmulsiFlex-C50 high pressure homogenizer at 15,000 psi and 15° C. for 30 passes through the adjustable homogenizing valve and collected in a 50 ml conical tube. The size of the particles produced was determined using photon correlation spectroscopy and the surface charge of the particles was also determined using micro-electrophoresis. See Table 32.

Method 2

15 ml of the poloxamer solution at 18 mg/ml in sterile water for injection and 15 ml of 2.4 mM DMRIE (6 mM stock diluted with sterile water for injection) were mixed in a 50 ml conical tube by gentle inversion at room temperature. The solution was then homogenized in an EmulsiFlex-C50 high pressure homogenizer at 15,000 psi and 15° C. for 30 passes through the adjustable homogenizing valve and collected in a 50 ml conical tube. The size of the particles produced was determined using photon correlation spectroscopy and the surface charge of the particles was determined using micro-electrophoresis. See Table 32. The formulation was then diluted with 50% sucrose solution to give 15 mg/ml CRL-1005, 2 mM DMRIE in 8.5% sucrose. The particle size and surface charge analysis was then repeated. See Table 32. When the 15 mg/ml CRL-1005, 2 mM DMRIE in 8.5% sucrose formulation was cooled below the cloud point of the poloxamer and then allowed to warm to room temperature, visible aggregates were present in the formulation.

TABLE 32

| | Particle size nm | Surface charge |
|---|---|---|
| Formulation | (polydispersity) | (mV) |
| 7.5 mg/ml CRL-1005 | 154 (0.36) | +39.5 |
| 1.0 mM DMRIE in 8.5% sucrose | | |
| Method 1 | | |
| 9 mg/ml CRL-1005 | 171 (0.11) | +37.2 |
| 1.2 mM DMRIE in SWFI | | |
| Method 2 | | |
| 15 mg/ml CRL-1005 | 162 (0.13) | +44.9 |
| 1.0 mM DMRIE in 8.5% sucrose | | |
| Method 2 | | |

Three different lyophilization procedures were conducted using a 7.5 mg/ml CRL-1005, 1.0 mM DMRIE in 8.5% sucrose formulation mixed according to Method 2.

Lyophilization Method 1

Two 10 ml borosilicate vials (Wheaton) were filled with 1 ml each of the formulation and the vials placed in a computer controlled Virtis Advantage freeze dryer at room temperature. Initially the vials were cooled below −40° C. for at least two hours and then the condenser was cooled to below 40° C. and the vacuum reduced to below 300 mTorr. The first step in primary drying was to hold the vials at 40° C. for one hour, under a vacuum of 120 mtorr. Then the temperature was raised to 20° C. over eight hours and the vacuum maintained at 120 mTorr. After eight hours the temperature and vacuum were maintained for an additional hour. The secondary drying step involved raising the temperature to 30° C. over 30 minutes and holding this temperature for two hours, while maintaining a vacuum of 120 mTorr. Finally the temperature was reduced to 20° C. over 30 minutes, the vials were sealed with grey butyl rubber stoppers (WestDirect) under vacuum and the samples removed for analysis.

One of the lyophilized samples was then reconstituted with 960 μl of sterile water for injection and gently mixed by hand and left on the bench top for 15 minutes. A 20 μl aliquot of the solution was then removed and diluted in 2 ml of filtered (0.2 μm) 10% sucrose and the particle size determined. See Table 33.

Lyophilization Method 2

Two 10 ml borosilicate vials (Wheaton) were filled with 1 ml each of the formulation and the vials placed in a computer controlled Virtis Advantage freeze dryer at a temperature of −65° C. Initially the vials were cooled below −40° C. for at least two hours and then the condenser was cooled to below −40° C. and the vacuum reduced to below 300 mTorr. The first step in primary drying was to hold the vials at 40° C. for one hour, under a vacuum of 120 mTorr. Then the temperature was raised to 20° C. over eight hours and the vacuum maintained at 120 mTorr. After eight hours the temperature and vacuum were maintained for an additional hour. The secondary drying step involved raising the temperature to 30° C. over 30 minutes and holding this temperature for a two hours, while maintaining a vacuum of 120 mTorr. Finally the temperature was reduced to 20° C. over 30 minutes, the vials were sealed with grey butyl rubber stoppers (WestDirect) under vacuum and the samples removed for analysis.

One of the lyophilized samples was then reconstituted with 960 μl of sterile water for injection and gently mixed by hand and left on the bench top for 15 minutes. A 20 μl aliquot of the solution was then removed and diluted in 2 ml of filtered (0.2 μm) 10% sucrose and the particle size determined. See Table 33.

Lyophilization Method 3

Two 10 ml borosilicate vials (Wheaton) were filled with 1 ml each of the formulation and then flash frozen in liquid nitrogen. The vials were then placed in a computer controlled Virtis Advantage freeze dryer at a temperature of −65° C. Initially the vials were kept below −40° C. for at least two hours and then the condenser was cooled to below −40° C. and the vacuum reduced to below 300 mTorr. The first step in primary drying was to hold the vials at −40° C. for one hour, under a vacuum of 120 mTorr. Then the temperature was raised to 20° C. over eight hours and the vacuum maintained at 120 mTorr. After eight hours the temperature and vacuum were maintained for an additional hour. The secondary drying step involved raising the temperature to 30° C. over 30 minutes and holding this temperature for two hours, while maintaining a vacuum of 120 mTorr. Finally the temperature was reduced to 20° C. over 30 minutes, the vials were sealed with grey butyl rubber stoppers (WestDirect) under vacuum and the samples removed for analysis.

One of the lyophilized samples was then reconstituted with 960 μl of sterile water for injection and gently mixed by hand and left on the bench top for 15 minutes. A 20 μl aliquot of the solution was then removed and diluted in 2 ml of filtered (0.2 μm) 10% sucrose and the particle size determined. See Table 33.

TABLE 33

| | Particle size nm | Particle size nm | Particle size nm | Surface charge |
|---|---|---|---|---|
| | (polydispersity) | (polydispersity) | (polydispersity) | (mV) |
| Formulation | Method 1 | Method 2 | Method 3 | Method 3 |
| 7.5 mg/ml CRL-1005 | 507 (0.93) | 348 (0.49) | 213 (0.27) | +45.4 |
| 1.0 mM DMRIE | | | | |
| in 8.5% sucrose | | | | |
| Method 2 | | | | |

The experiments of this example demonstrate that when certain poloxamer solutions were subjected to high pressure homogenization in the presence of DMRIE, small uniform particles were produced with a positive surface charge. When these particles were suspended in 8.5% sucrose, they could be lyophilized under certain conditions. The particles could also be reconstituted with water to produce cell delivery particles physically comparable to those that had not been lyophilized.

### Example 10

This example describes the change in particle size and surface charge when poloxamer and VC1052 solutions, with out without DNA, are subjected to homogenization, lyophilized and then reconstituted.

Stock solutions of 6 mM VC1052 in sterile water for injection (SWFI) and 30 mg/ml CRL-1005 in sterile water for injection were made. The CRL-1005 stock solution was diluted with sterile water for injection to 18 mg/ml and the 6 mM VC1052 stock was diluted with sterile water for injection to 2.4 mM. 15 ml of the poloxamer solution (18 mg/ml) was mixed with 15 ml of the 2.4 mM VC1052 lipid solution in a 50 ml conical tube by gentle inversion at room temperature. The solution was then homogenized in an EmulsiFlex-C50 high pressure homogenizer at 15,000 psi and 15° C. for 30 passes through the adjustable homogenizing valve and collected in a 50 ml conical tube. The size of the particles produced was determined using photon correlation spectroscopy. See Table 34. The formulation was then diluted with 50% sucrose solution to give 7.5 mg/ml CRL-1005, 1 mM VC1052 in 8.5% sucrose. The particle size analysis was then repeated. See Table 34.

TABLE 34

| | Particle size nm |
|---|---|
| Formulation | (polydispersity) |
| 9.0 mg/ml CRL-1005 | 112 (0.31) |
| 1.2 mM VC1052 | |
| in SWFI | |
| 7.5 mg/ml CRL-1005 | 91 (0.61) |
| 1.0 mM VC1052 | |
| in 8.5% sucrose | |

Two 10 ml borosilicate vials (Wheaton) were filled with 1 ml each of the sucrose formulation, as shown in Table 34, and then flash frozen in liquid nitrogen. The vials were then placed in a computer controlled Virtis Advantage freeze dryer at a temperature of −65° C. Initially the vials were kept below −40° C. for at least two hours and then the condenser was cooled to below −40° C. and the vacuum reduced to below 300 mTorr. The first step in primary drying was to hold the vials at −40° C. for one hour, under a vacuum of 120 mTorr. Then the temperature was raised to 20° C. over eight hours and the vacuum maintained at 120 mTorr. After eight hours the temperature and vacuum were maintained for an additional hour. The secondary drying step involved raising the temperature to 30° C. over 30 minutes and holding this temperature for two hours, while maintaining a vacuum of 120 mTorr. Finally the temperature was reduced to 20° C. over 30 minutes, the vials were sealed with grey butyl rubber stoppers (WestDirect) under vacuum and the samples removed for analysis. One of the lyophilized samples was then reconstituted with 960 μl of sterile water for injection and gently mixed by hand and left on the bench top for 15 minutes. The size of the particle produce was determined using photon correlation spectroscopy and the surface charge of the particles was also determined using micro-electrophoresis. See Table 35.

A 1 ml aliquot of the 7.5 mg/ml CRL-1005, 1 mM VC1052 particles in 8.5% sucrose solution at room temperature were put into 10 ml borosilicate vials (Wheaton). Plasmid DNA was then added using a 100 μl pipette to give a charge ratios of 0.5:

1. 25.3 μl of 6.58 mg/ml DNA=166 μg or 0.5 charge ratio (−/+)

The solutions were then mixed by gentle inversion 5 times, incubated at room temperature for 30 minutes and then flash frozen in liquid nitrogen. The vials were then placed in a computer controlled Virtis Advantage freeze dryer at a temperature of −65° C. and the lyophilization procedure described above repeated. One of the lyophilized samples was then reconstituted with 960 μl of sterile water for injection and gently mixed by hand and left on the bench top for 15 minutes. The size of the particle produce was determined using photon correlation spectroscopy and the surface charge of the particles was also determined using micro-electrophoresis. See Table 35.

TABLE 35

| | Particle size nm | Surface charge |
|---|---|---|
| | (polydispersity) | (mV) |
| Formulation | Lyo method 3 | Lyo method 3 |
| 7.5 mg/ml CRL-1005 | 152 (0.20) | +50.2 |
| 1.0 mM VC1052 | | |
| in 8.5% sucrose | | |
| Method 3 | | |
| 7.5 mg/ml CRL-1005 | 195 (0.17) | +44.3 |
| mM VC1052 and 166 μg | | |
| DNA in 8.5% sucrose | | |
| Method 3 | | |

The experiments of this example demonstrate that when certain poloxamer solutions are subjected to high pressure homogenization in the presence of VC1052, small uniform particles are produced with a positive surface charge. When these particles are in 8.5% sucrose, with or without DNA, they can be lyophilized and reconstituted with water to produce stable cell delivery particles.

### Example 11

This example describes the changes in particle size and surface charge when poloxamer and DMRIE:DOPE solutions were subject to high pressure homogenization.

A 9 mg/ml solution of DMRIE:DOPE (1:1 molar ratio) was made (6.53 mM DMRIE solution for charge ration calculations) in PBS. The solution was homogenized and extruded in an EmulsiFlex-C50 high pressure homogenizer fitted with an optional filter/extruder (F) down stream from the homogenizer valve. See FIG. 4. The solution was processed at 10,000 psi and 15° C. for 5 passes through the adjustable homogenizing valve. The extruder was fitted with three 50 nm pore size filter membranes and the solution was collected after processing in a 50 ml conical tube. Particle size was measured by photon correlation spectroscopy. See Table 36. A stock solution of 15 mg/ml CRL-1005 in PBS was made as described in the methods section.

The following formulations were then made by homogenization in an EmulsiFlex-C50 high pressure homogenizer at 15,000 psi and 15° C. for 30 passes through the adjustable homogenizing valve and collected in 50 ml conical tubes:

1. 0.375 mg/ml CRL-1005+0.075 mM DMRIE: DOPE (1:1)

2. 1.5 mg/ml CRL-1005+0.075 mM DMRIE: DOPE (1:1)

3. 3.75 mg/ml CRL-1005+0.075 mM DMRIE: DOPE (1:1)

The size of the particles produced was determined using photon correlation spectroscopy and the surface charge of the particles was also determined using micro-electrophoresis. See Table 37.

TABLE 36

| | Particle size nm |
|---|---|
| Formulation | (polydispersity) T = 2 h |
| 10 mg/ml DMRIE:DOPE | 78 (0.21) |
| in PBS | |

TABLE 37

| | Particle size nm | Surface charge |
|---|---|---|
| | (polydispersity) | (mV) |
| Formulation | Lyo method 1 | Lyo method 3 |
| 0.375 mg/ml CRL-1005 | 186 (0.48) | +15.8 |
| 1.0 mM DM:DP (1:1) | | |
| in PBS | | |
| 1.5 mg/ml CRL-1005 | 147 (0.25) | +11.0 |
| 1.0 mM DM:DP (1:1) | | |
| in PBS | | |
| 3.75 mg/ml CRL-1005 | 156 ((0.16) | +2.7 |
| 1.0 mM DM:DP (1:1) | | |
| in PBS | | |

The experiment of this example demonstrates that when certain poloxamer solutions were subjected to high pressure homogenization in the presence of DMRIE:DOPE, small uniform particles were produced with a positive surface charge.

### Example 12

This example describes the changes in particle size and surface charge when poloxamer and BAK C₁₈ homolog solutions were subject to high pressure homogenization at different temperatures. The changes in particle size and surface charge of the homogenized particles after the addition of DNA is also described.

Stock solutions of 6.28 mg/ml CRL-1005 in PBS and 2 mM BAK C₁₈ in PBS were made. 22.5 ml of the poloxamer solution at 8.37 mg/ml and 7.5 ml of the 2.0 mM lipid solution (warmed to 45° C. prior to use) were then mixed in a 50 ml conical tube by gentle inversion at room temperature. The solution was then homogenized in an EmulsiFlex-C50 high pressure homogenizer at 15,000 psi and 15° C. for 30 passes through the adjustable homogenizing valve and collected in a 50 ml conical tube. The size of the particles produced was determined using photon correlation spectroscopy and the surface charge of the particles was also determined using micro-electrophoresis. See Table 38.

22.5 ml of the poloxamer solution at 8.37 mg/ml and 7.5 ml of the 2.0 mM lipid solution (warmed to 45° C. prior to use) were then mixed in a 50 ml conical tube by gentle inversion at room temperature. The solution was then homogenized in an EmulsiFlex-C50 high pressure homogenizer at 15,000 psi and 37° C. for 30 passes through the adjustable homogenizing valve and collected in a 50 ml conical tube. The tube was then gently mixed by rotation for 1 hour until the formulation had cooled to room temperature. The size of the particles produced was determined using photon correlation spectroscopy and the surface charge of the particles was also determined using micro-electrophoresis. See Table 38.

TABLE 38

| | | Polydispersity | |
|---|---|---|---|
| Homogenization | Particle | of particle | Surface |
| temperature (° C.) | size (nm) | size distribution | charge (mV) |
| 15 | 362 | 0.50 | +4.3 |
| 37 | 264 | 0.21 | +38.5 |

403 μl of 6.2 mg/ml plasmid was placed into a 2 ml glass vial at room temperature and 597 μl of the 37° C. homogenized CRL-1005+BAK C₁₈ solution was added using a 1 ml pipette, the vial was sealed with a rubber stopper and mixed my inversion 5 times. The size of the particles produced was determined using photon correlation spectroscopy and the surface charge of the particles was also determined using micro-electrophoresis. See Table 39.

403 μl of 6.2 mg/ml plasmid was placed into a 2 ml glass vial at room temperature and 597 μl of the 37° C. homogenized CRL-1005+BAK C₁₈ solution was added rapidly using a 1 ml syringe an 22 gauge needle, the vial was sealed with a rubber stopper and mixed my inversion 5 times. The size of the particles produced was determined using photon correlation spectroscopy and the surface charge of the particels was also determined using micro-electrophoresis. See Table 39.

TABLE 39

| | | Polydispersity | |
|---|---|---|---|
| | Particle | of particle | |
| Mixing method | size (nm) | size distribution | Surface charge (mV) |
| Pipette | 424 | 0.93 | +44.3 |
| Needle and | 329 | 0.28 | +43.6 |
| syringe | | | |

The experiments of this example demonstrate that when certain poloxamer solutions are subjected to high pressure homogenization in the presence of the BAK C₁₈ homolog, small uniform particles are produced with a positive surface charge at a temperature of about 37° C. Stable formulations using the cell delivery particles produced in this example and DNA could only be produced via rapid addition of the DNA to the particles with a needle and syringe.

### Example 13

This example describes the interactions of poloxamer CRL-1005 and the BAK C₁₈ homolog using the thermal cycling methods described in U.S. Published Patent Applications 2004/0162256 A1 and 2004/0209241 A1.

8.5 ml of 4.41 mg/ml CRL-1005 in PBS was placed in a 25 ml round bottom flask and stirred for 30 minutes on ice. The ice bath was then removed, the solution stirred at ambient temperature for 15 minutes to produce a cloudy solution as the poloxamer passed through the cloud point. The flask was then placed back into the ice bath and stirred for a further 15 minutes to produce a clear solution as the mixture cooled below the poloxamer cloud point. The ice bath was again removed and the solution stirred for a further 15 minutes. Stirring for 15 minutes above and below the cloud point (total of 30 minutes), was defined as one thermal cycle. The mixture was cycled three more times. The size of the particles produced was determined using photon correlation spectroscopy and their surface charge was also determined using micro-electrophoresis. See Table 40. The stirring solution was then warmed to 37° C. in a water bath and 1.5 ml of 2 mM BAK C₁₈ in PBS was added via a 1 ml pipette. The final concentrations of the solution were 3.75 mg/ml CRL-1005 and 0.3 mM BAK C₁₈ in PBS. The solution was stirred for 20 minutes at 37° C. and then cooled to room temperature and stirred for a total of one hour. The size of the particles produced was determined using photon correlation spectroscopy and their surface charge was also determined using micro-electrophoresis. See Table 40.

TABLE 40

| | Particle size nm | |
|---|---|---|
| Formulation | (polydispersity) | Surface charge mV |
| 4.41 mg/ml CRL-1005 | 826 (0.89) | −0.4 +/− 10.6 |
| in PBS | | |
| 3.75 mg/ml CRL-1005 | 705 (0.50) | +29.1 +/− 25.9 |
| 0.30 mM BAK C₁₈ | | |
| in PBS | | |

The experiment of this example demonstrates that the BAK C₁₈ homolog does not form small nor uniform cell delivery particles with CRL-1005 when using the thermal cycling method described in U.S. Published Patent Applications 2004/0162256 A1 and 2004/0209241 A1.

### Example 14

This example describes the in vivo biological activity of cell delivery particle formulations containing DNA, made by homogenizing poloxamer and DMRIE:Cholesterol or Vaxfectin™ solutions.

30 ml of a 2.36 mM DMRIE:Cholesterol (1:1 molar ratio) solution was made in sterile water for injection and was homogenized and extruded in an EmulsiFlex-C50 high pressure homogenizer fitted with an optional filter/extruder (F) down stream from the homogenizer valve. See FIG. 4. The solution was processed at 10,000 psi and 15° C. for 5 passes through the adjustable homogenizing valve. The extruder was fitted with three 50 nm pore size filter membranes and the liposome solution was collected after processing in a 50 ml conical tube

35 ml of a 4.35 mM Vaxfectin™ solution was made in sterile water for injection and was homogenized and extruded in an EmulsiFlex-C50 high pressure homogenizer fitted with an optional filter/extruder (F) down stream from the homogenizer valve. See FIG. 4. The solution was processed at 10,000 psi and 15° C. for 5 passes through the adjustable homogenizing valve. The extruder was fitted with three 50 nm pore size filter membranes and the liposome solution was collected after processing in a 50 ml conical tube.

100 ml of 15 mg/ml CRL-1005 solution was made as described in the general experimental section. The following stock solutions were then made in 50 ml conical tubes.

1. 4 mg/ml CRL-1005+1.5 mM DMRIE:Cholesterol

2. 4 mg/ml CRL-1005+0.15 mM DMRIE:Cholesterol

3. 4 mg/ml CRL-1005+1.5 mM Vaxfectin™

4. 4 mg/ml CRL-1005+0.15 mM Vaxfectin™

The solutions were then homogenized in an EmulsiFlex-C50 high pressure homogenizer at 15,000 psi and 15° C. for 30 passes through the adjustable homogenizing valve and collected in a 50 ml conical tube. The size of the particles produced was determined using photon correlation spectroscopy (See Table 41) and the zeta potential was measured using microelectrophoresis (See Table 41).

TABLE 41

| | Particle size (nm) | |
|---|---|---|
| | after | Surface |
| | homogenization | charge (mV) after |
| Formulation | (polydispersity) | homogenization |
| 4.0 mg/ml CRL-1005 | 110 (0.33) | +22.7 |
| 0.15 mM DMRIE:Chol (1:1) in | | |
| PBS | | |
| 4.0 mg/ml CRL-1005 | 106 (0.33) | +28.6 |
| 1.5 mM DMRIE:Chol (1:1) | | |
| in PBS | | |
| 4.0 mg/ml CRL-1005 | 113 (0.27) | +23.0 |
| 0.15 mM Vaxfectin | | |
| in PBS | | |
| 4.0 mg/ml CRL-1005 | 86 (0.33) | +28.1 |
| 1.5 mM Vaxfectin | | |
| in PBS | | |

In Vivo Analysis of Formulation

The following groups were then made for in vivo testing:

| Group | | DNA (total/injection/mouse) | # of Mice |
|---|---|---|---|
| A | BALB/c | 10 μg VR4700 (5 μg/50 μl PBS/leg) | 9 |
| B | BALB/c | 10 μg VR4700 + VF-P1205-02A | 9 |
| C | BALB/c | 10 μg VR4700 + CRL1005 0.2 mg/ml + | 9 |
| | | DM:Chol (4:1, −/+) | |
| D | BALB/c | 10 μg VR4700 + CRL1005 2.0 mg/ml + | 9 |
| | | DM:Chol (4:1, −/+) | |
| E | BALB/c | 10 μg VR4700 + CRL1005 0.2 mg/ml + | 9 |
| | | Vaxfectin (4:1, −/+) | |
| F | BALB/c | 10 μg VR4700 + CRL1005 2.0 mg/ml + | 9 |
| | | Vaxfectin (4:1, −/+) | |

Group C was made by placing 0.2 ml of stock solution #1 (4 mg/ml CRL-1005+1.5 mM DMRIE:Cholesterol) in a 10 ml glass vial, then adding 0.4 ml of 10× PBS and 1.8 ml of sterile water for injection and mixing the resulting solution by gentle inversion 5 times. This solution was then added by pipette to 1.6 ml of VR4700 at 0.25 mg/ml in PBS and the solution mixed by gentle inversion five times. Particle size was measured using photon correlation spectroscopy (See Table 42) and zeta potential was measured using microelectrophoresis (See Table 42).

Group E was made by placing 0.2 ml of stock solution #3 (4 mg/ml CRL-1005+1.5 mM Vaxfectin™) in a 10 ml glass vial, then adding 0.4 ml of 10× PBS and 1.8 ml of sterile water for injection and mixing the resulting solution by gentle inversion 5 times. This solution was then added by pipette to 1.6 ml of VR4700 at 0.25 mg/ml in PBS and the solution mixed by gentle inversion five times. Particle size was measured using photon correlation spectroscopy (See Table 42) and zeta potential was measured using microelectrophoresis (See Table 42).

Group D was made by placing 2.0 ml of stock solution #2 (4 mg/ml CRL-1005+0.15 mM DMRIE:Cholesterol) in a 10 ml glass vial, then adding 0.4 ml of 10× PBS and mixing the resulting solution by gentle inversion 5 times. This solution was then added by pipette to 1.6 ml, of VR4700 at 0.25 mg/ml in PBS and the solution mixed by gentle inversion five times. Particle size was measured using photon correlation spectroscopy (See Table 42) and zeta potential was measured using microelectrophoresis (See Table 42).

Group F was made by placing 2.0 ml of stock solution #4 (4 mg/ml CRL-1005+0.15 mM Vaxfectin™) in a 10 ml glass vial, then adding 0.4 ml of 10× PBS and mixing the resulting solution by gentle inversion 5 times. This solution was then added by pipette to 1.6 ml of VR4700 at 0.25 mg/ml in PBS and the solution mixed by gentle inversion five times. Particle size was measured using photon correlation spectroscopy (See Table 42) and zeta potential was measured using microelectrophoresis (See Table 42).

Group A was a naked DNA control, and group B was a thermally cycled DNA/CRL-1005/BAK or CRL-1005 02A (5 mg/ml DNA, 7.5 mg/ml CRL-1005, and 0.3 mM BAK) formulation.

TABLE 42

| | Particle size | |
|---|---|---|
| | (nm) after | |
| | homogenization | Surface charge (mV) |
| Formulation | (polydispersity) | after homogenization |
| 0.2 mg/ml CRL-1005 | 226 (0.22) | −25.4 |
| 0.075 mM DMRIE:Chol (1:1) | | |
| 0.1 mg/ml VR4700 | | |
| 2.0 mg/ml CRL-1005 | 158 (0.30) | −23.2 |
| 0.075 mM DMRIE:Chol (1:1) | | |
| 0.1 mg/ml VR4700 | | |
| 0.2 mg/ml CRL-1005 | 260 (0.35) | −33.9 |
| 0.075 mM Vaxfectin ™ | | |
| 0.1 mg/ml VR4700 | | |
| 2.0 mg/ml CRL-1005 | 214 (0.22) | −23.6 |
| 0.075 mM Vaxfectin ™ | | |
| 0.1 mg/ml VR4700 | | |

BALB/c female mice (9/group, 54 mice total) were given bilateral intramuscular injections into the rectus femoris with 5 μg dose of plasmid DNA in 50 μl per leg. Mice received injections on days 0, 20 and 48. OSP bleeds were taken on day 61 and splenocytes were harvested on days 62, 63 and 64. NP-specific antibodies were analyzed by ELISA (See Tables 43 and 44) and NP-specific Th and Tc cells were analyzed by IFN-γ ELISPOT (See Tables 45 and 46) as described in the general methods section.

The experiments of this example demonstrate that when DNA is incubated with poloxamer and DMRIE:Cholesterol or Vaxfectin™ cell delivery particles, formulations comprising the resulting particles are biologically active in vivo.

TABLE 43

| 9 W Anti-NP Titers | | | | |
|---|---|---|---|---|
| n = 9 mice/group | Avg | | s.e.m. | Fold x |
| 10 μg VR4700, (5 μg/50 μl/leg) | 27,022 | ± | 4,978 | 1.0 |
| VR4700 + VF-P1205-02A | 68,267 | ± | 8,533 | 2.5 |
| +CRL-1005 0.2 mg/ml + DM:Ch | 39,822 | ± | 8,651 | 1.5 |
| +CRL-1005 2 mg/ml + DM:Ch | 46,933 | ± | 8,533 | 1.7 |
| +CRL-1005 0.2 mg/ml + Vaxfectin | 65,422 | ± | 12,151 | 2.4 |
| +CRL-1005 2 mg/ml + Vaxfectin | 62,578 | ± | 10,547 | 2.3 |

TABLE 44
NP specific Serum Ab Titers (total IgG)

| | 3 W | | 6 W | | |
|---|---|---|---|---|---|
| | Titers | | Titers | | 9 W Titers |
| n = 9 mice/group | Avg | s.e.m. | Avg | s.e.m. | Avg s.e.m. |
| 10 μg VR4700 | 6,542 | 965 | 31,289 | 5,274 | 27,022 ± 4,978 |
| (5 μg/50 μl/leg) | | | | | |
| VR4700 + VF-P1205-02A | 13,084 | 3,898 | 56,889 | 9,326 | 68,267 ± 8,533 |
| +CRL-1005 0.2 mg/ml + DM:Ch | 6,258 | 753 | 34,133 | 9,299 | 39,822 ± 8,651 |
| +CRL-1005 2 mg/ml + DM:Ch | 6,827 | 1,707 | 25,600 | 3,695 | 46,933 ± 8,533 |
| +CRL-1005 0.2 mg/ml + Vaxfectin | 10,524 | 2,109 | 52,622 | 10,547 | 65,422 ± 12,151 |
| +CRL-1005 2 mg/ml + Vaxfectin | 7,680 | 1,045 | 35,556 | 9,724 | 62,578 ± 10,547 |

TABLE 45
CD8⁺ IFN-γ ELISPOT Data

| | Spleen Harvest # | | | | |
|---|---|---|---|---|---|
| CD8 ELISPOT Average | 1 | 2 | 3 | Avg s.e.m. | Fold x |
| 10 μg VR4700 | 52 | 31 | 65 | 49 ± 10 | 1.0 |
| (5 μg/50 μl/leg) | | | | | |
| VR4700 + | 125 | 148 | 208 | 161 ± 25 | 3.3 |
| VF-P1205-02A | | | | | |
| +CRL-1005 0.2 mg/ | 187 | 180 | 129 | 165 ± 18 | 3.4 |
| ml + DM:Ch | | | | | |
| +CRL-1005 2 mg/ | 77 | 201 | 213 | 164 ± 43 | 3.3 |
| ml + DM:Ch | | | | | |
| +CRL-1005 0.2 mg/ | 115 | 108 | 155 | 126 ± 15 | 2.6 |
| ml + Vaxfectin | | | | | |
| +CRL-1005 2 mg/ml + | 29 | 22 | 30 | 27 ± 3 | 0.5 |
| Vaxfectin | | | | | |

TABLE 46
CD4⁺ IFN-γ ELISPOT Data

| | Spleen Harvest # | | | | |
|---|---|---|---|---|---|
| CD4 ELISPOT Average | 1 | 2 | 3 | Avg s.e.m. | Fold x |
| 10 μg VR4700 (5 μg/ | 37 | 20 | 39 | 32 ± 6 | 1.0 |
| 50 μl/leg) | | | | | |
| VR4700 + VF-P1205-02A | 85 | 53 | 170 | 102 ± 35 | 3.2 |
| +CRL-1005 0.2 mg/ml + | 73 | 151 | 241 | 155 ± 48 | 4.9 |
| DM:Ch | | | | | |
| +CRL-1005 2 mg/ml + | 76 | 135 | 112 | 108 ± 17 | 3.4 |
| DM:Ch | | | | | |
| +CRL-1005 0.2 mg/ml + | 46 | 85 | 154 | 95 ± 32 | 3.0 |
| Vaxfectin | | | | | |
| +CRL-1005 2 mg/ml + | 7 | 4 | 7 | 6 ± 1 | 0.2 |
| Vaxfectin | | | | | |

1-117. (canceled)

118. Cell delivery particles manufactured by a method comprising: homogenizing, in an aqueous solution, a mixture comprising:
(i) an amphiphilic component comprising an amphiphile selected from the group consisting of: a cationic amphiphile, an anionic amphiphile, a zwitterionic amphiphile or any combination thereof and
(ii) a polyoxyethylene (POE) and polyoxypropylene (POP) block copolymer; to form a homogenate, wherein said homogenate is in the form of particles.

119. The cell delivery particles of claim 118, wherein said amphiphilic component and said block copolymer are mixed together prior to said homogenization.

120. The cell delivery particles of claim 118, wherein said amphiphilic component and said block copolymer are mixed together simultaneously with said homogenization.

121. The cell delivery particles of claim 118, wherein said block copolymer is of the general formula:
HO(C₂H₄O)ₓ(C₃H₆O)_{y}(C₂H₄O)ₓH; wherein (y) represents a number such that the molecular weight of the hydrophobic POP portion (C₃H₆O) is in a range from about 1000 daltons_up to approximately 20,000 daltons and wherein (x) represents a number such that the percentage of the hydrophilic POE portion (C₂H₄O) is between approximately 1% and 50% by weight.

122. The cell delivery particles of claim 121, wherein said block copolymer is the poloxamer CRL-1005.

123. The cell delivery particles of claim 118, wherein the amphiphilic component comprises a cationic amphiphile selected from the group consisting of benzalkonium chloride, benethonium chloride, cetrimide, cetylpyridinium chloride, acetyl triethylammonium chloride, (±)-N-(2-hydroxyethyl)-N,N-dimethyl-2,3-bis (tetradecyloxy)-1-(propanaminium bromide) (DMRIE), (±)-N-(3-aminopropyl)-N,N-dimethyl-2,3-bis(syn-9-tetradeceneyloxy)-1-propanaminium bromide (VC1052), (±)-N-(Benzyl)-N,N-dimethyl-2,3-bis(hexyloxy)-1-propanaminium bromide (Bn-DHxRIE), (±)-N-(2-Acetoxyethyl)-N,N-dimethyl-2,3-bis(hexyloxy)-1-propanaminium bromide (DHxRIE-OAc), (±)-N-(2-Benzoyloxyethyl)-N,N-dimethyl-2,3-bis(hexyloxy)-1-propanaminium bromide (DHxRIE-OBz), (±)-N-(3-acetoxypropyl)-N,N-dimethyl-2,3-bis(octyloxy)-1-propanaminium chloride (Pr-DOctRIE-OAc), (±)-N-(3-aminopropyl)-N,N-dimethyl-2,3-bis(decyloxy)-1-propanaminium bromide (GAP-DDRIE) or mixtures of two or more of said cationic amphiphiles.

124. The cell delivery particles of claim 123, wherein the cationic amphiphile comprises benzalkonium chloride.

125. The cell delivery particles of claim 123, wherein the cationic amphiphile comprises DMRIE.

126. The cell delivery particles of claim 123, wherein the cationic amphiphile comprises VC1052.

127. The cell delivery particles of claim 123, wherein said mixture further comprises a co-lipid.

128. The cell delivery particles of claim 127, wherein said co-lipid is selected from the group consisting of: 1,2-dioleoyl-sn-glycero-3-phosphoethanolamine (DOPE), 1,2-diphytanoyl-sn-glycero-3-phosphoethanolamine (DPyPE), 1,2-dimyristoyl-glycer-3-phosphoethanolamine (DMPE), cholesterol or mixtures thereof.

129. The cell delivery particles of claim 123, wherein the block copolymer comprises CRL-1005.

130. The cell delivery particles of claim 118, wherein said aqueous solution comprises a pH stabilizing suitable physiological solution.

131. The cell delivery particles of claim 130, wherein said suitable physiological solution comprises a phosphate anion solution.

132. The cell delivery particles of claim 118, wherein said homogenization is performed at a temperature of about 10° C. to about 90° C.

133. The cell delivery particles of claim 119, wherein the mixing is performed at a temperature of about 10° C. to about 90° C. and said homogenization is performed at a temperature of about 10° C. to about 90° C.

134. The cell delivery particles of claim 118 which are sterile.

135. A pharmaceutical composition produced by a method comprising mixing the cell delivery particles of claim 118 with a pharmaceutical component selected from the group consisting of: a pharmaceutically active drug, an antigenic molecule, a polynucleotide, or any combination thereof.

136. The pharmaceutical composition of claim 135 which is sterile.

137. The pharmaceutical composition of claim 136, wherein said homogenization is performed under sterile conditions.

138. The pharmaceutical composition of claim 135, wherein the final concentration of said amphiphilic component present in said pharmaceutical composition is from about 0.001 mM to about 10 mM and wherein the final concentration of said block copolymer present in said pharmaceutical composition is from about 0.01 mg/mL to about 50 mg/mL.

139. The pharmaceutical composition of claim 135, wherein said pharmaceutical component is a polynucleotide and wherein the final concentration of said polynucleotide in said pharmaceutical composition is from about 1 ng/mL to about 10 mg/mL.

140. The pharmaceutical composition of claim 139, wherein said method further comprises lyophilization and wherein said lyophilization comprises a first drying step at a temperature of about −80° C. to about −20° C. and a second drying step at a temperature of about 10° C. to about 40° C.

141. The pharmaceutical composition of claim 139, wherein said polynucleotide is selected from the group consisting of: DNA, RNA or a combination thereof.

142. The pharmaceutical composition of claim 141, wherein said polynucleotide is DNA.

143. The pharmaceutical composition of claim 141, wherein said polynucleotide is RNA.

144. The pharmaceutical composition of claim 135, wherein said pharmaceutical component is a polynucleotide, and wherein said polynucleotide encodes a polypeptide.

145. The pharmaceutical composition of claim 144, wherein said polynucleotide operably expresses said polypeptide in eukaryotic cells.

146. A composition comprising cell delivery particles of claim 118, comprising:
(i) an amphiphilic component comprising an amphiphile selected from the group consisting of: DMRIE, VC1052, N-benzyl-N,N-dimethyl-N-dodecyl-ammonium chloride, N-benzyl-N,N-dimethyl-N-teradecyl-ammonium chloride, N-benzyl-N,N-dimethyl-N-hexadecyl-ammonium chloride, N-benzyl-N,N-dimethyl-N-octadecyl-ammonium chloride or a combination thereof and
(ii) CRL-1005.

147. A method for generating a detectable immune response comprising administering to a vertebrate the pharmaceutical composition of claim 144, wherein said composition is administered in an amount sufficient to elicit a detectable immune response to a polypeptide encoded by said polynucleotide.

148. A method to treat or prevent a disease or condition in a vertebrate comprising: administering to a vertebrate in need thereof the pharmaceutical composition of claim 135.

149. The method of claim 147, wherein said vertebrate is a mammal.

150. The method of claim 149, wherein said mammal is a human.

151. The method of claim 147, wherein said pharmaceutical composition is administered to a tissue selected from the group consisting of muscle, skin, brain tissue, lung tissue, liver tissue, spleen tissue, bone marrow tissue, thymus tissue, heart tissue, lymph tissue, blood tissue, bone tissue, connective tissue, mucosal tissue, pancreas tissue, kidney tissue, gall bladder tissue, intestinal tissue, testicular tissue, ovarian tissue, uterine tissue, vaginal tissue, rectal tissue, nervous system tissue, eye tissue, glandular tissue, and tongue tissue.

152. The method of claim 147, wherein said pharmaceutical composition is administered to a cavity selected from the group consisting of the lungs, the mouth, the nasal cavity, the stomach, the peritoneal cavity, the intestine, a heart chamber, veins, arteries, capillaries, lymphatic cavities, the uterine cavity, the vaginal cavity, the rectal cavity, joint cavities, ventricles in brain, spinal canal in spinal cord, and the ocular cavities.

153. The method of claim 151, wherein said muscle is skeletal muscle, smooth muscle, or myocardium.

154. The method of claim 147, wherein said administration is by a route selected from the group consisting of intramuscular, intratracheal, intranasal, transdermal, interdermal, subcutaneous, intraocular, vaginal, rectal, intraperitoneal, intraintestinal, inhalation or intervenous.

155. The method of claim 154, wherein said administration is intramuscular.

156. A kit comprising the pharmaceutical composition of claim 135.


SEQUENCE LISTING (3 sequences)

SEQ ID NO: 1 - DNA, 30 bp, Artificial Sequence
  Chemically synthesized - TPROBE 04i
cattgcatccatgattgcttcacagcgtcc

SEQ ID NO: 2 - DNA, 21 bp, Artificial Sequence
  Chemically synthesized - forward primer
ccgtgccaagagtgactcacc

SEQ ID NO: 3 - DNA, 19 bp, Artificial Sequence
  Chemically synthesized - reverse primer
ctctagcgctgggcgaaac